(19) United States
(12) Patent Application Publication
(10) Pub. No.: US 2012/0190832 A1
(43) Pub. Date: Jul. 26, 2012

(54) LOW-MOLECULAR SERINE PROTEASES INHIBITORS COMPRISING POLYHYDROXY-ALKYL AND POLYHYDROXY-CYCLOALKYL RADICALS

(75) Inventors: Dieter Herr, Altrip (DE); Helmut Mack, Ludwigshafen (DE); Werner Seitz, Planckstadt (DE); Wilfried Hornberger, Neustadt (DE)

(73) Assignee: Abbott GmbH & Co. KG, Wiesbaden (DE)

(21) Appl. No.: 13/277,829

(22) Filed: Oct. 20, 2011

Related U.S. Application Data
(63) Continuation of application No. 12/850,545, filed on Aug. 4, 2010, now abandoned.
Application No. 12/850,545 is a continuation of application No. 10/398,269, filed on Aug. 18, 2003, now abandoned.

(30) Foreign Application Priority Data
Oct. 6, 2000 (DE) 10049937.6

(51) Int. Cl. C07H 15/26

(52) U.S. Cl. 536/17.4

(57) ABSTRACT

The invention relates to novel amidines and quanidines, the production and use thereof and the use thereof as trypsine-type serine protease competitive inhibitors, especially thrombine and compliment proteases CIs and C1r. The invention also relates to pharmaceutical compositions which contain said compounds as active ingredients, in addition to the use of the compounds as thrombine inhibitors, anticoagulants, compliment inhibitors and anti-inflammatory agents. The novel compositions are characterised by the linkage of a serine protease inhibitor having amidine or guanidine functions with an alkyl radical having two or more hydroxyl functions, whereby said alkyl radical is derived from sugar derivates. Several sugar structural components or components derived from sugar can therefore be linked to each other. Said principle of linking sugar derivates enables oral active compounds to be obtained.

The present invention relates to novel amidines and guanidines, to the production thereof, and to the use thereof as competitive inhibitors of trypsin-like serine proteases, particularly thrombin and the complement proteases C1s and C1r.

The invention also relates to pharmaceutical compositions containing said compounds as active ingredients, and also to the use of said compounds as thrombin inhibitors, anticoagulants, complement inhibitors, or anti-inflammatory agents. A characteristic of the novel compounds is their ability to link a serin protease inhibitor having an amidine or guanidine function to an alkyl group having two or more hydroxyl functions and derived from sugar derivatives. Thus a number of sugar building blocks or building blocks derived from sugars can be linked. This principle of coupling with sugar derivatives provides orally active compounds.

Preferred sugar derivatives include all types of reductive sugars which reductively react with a terminal amine function of the inhibitor.

Reductive sugars are sugars which are capable of reducing Cu(II) ions in solution to Cu(I) oxide.

Reductive sugars include:
Any of the aldoses (whether in open-chain or cyclic form) (eg, trioses; or tetraoses such as erythrose and threose; or pentoses such as arabinose, xylose, rhamnose, fucose, and ribose; or hexoses such as glucose, mannose, galactose, and 2-deoxy-D-glucose, etc.);
any of the (hydroxy)ketoses. Hydroxyketoses contain a HOCH₂—CO group. Fructose and ribulose are examples thereof.
Di-, oligo- and poly-saccharides containing a hemiacetal, such as lactose, melibiose, maltose, maltotriose, maltotetraose, maltohexaose, or cellulose oligomers such as cellobiose, cellotriose or dextran oligomers or pullulan oligomers or inulin oligomers, etc.
Sugar derivatives and complex oligosaccharides containing a hemiacetal, such as glucuronic acid, galacturonic acid, 2-deoxy-D-glucose, 2-deoxy-2-fluoro-D-glucose, glucosamine, N-acetyl-D-glucosamine, oligomers of pectin and hyaluronic acid.

Examples of other preferred sugar derivatives are sugar acids which react with a terminal amine function of the inhibitor via the acyl function.

Thrombin is a member of the group of serine proteases and plays a central role as terminal enzyme in the blood coagulation cascade. Both the intrinsic and the extrinsic coagulation cascades cause, via a number of intensification stages, the production of thrombin from prothrombin. Thrombin-catalyzed cleavage of fibrinogen to fibrin then triggers blood coagulation and aggregation of the thrombocytes, which in turn increase the formation of thrombin by binding platelet factor 3 and coagulation factor XIII as well as via a whole series of highly active mediators.

The formation and action of thrombin are central events in the genesis of both white arterial thrombi and red venous thrombi and are therefore potentially effective points of attack for pharmacological agents. Thrombin inhibitors are, unlike heparin, capably of completely inhibiting, simultaneously, the action of free thrombin and thrombin bound to thrombocytes, irrespective of co-factors. They can prevent, in the acute phase, thrombo-embolic events following percutane transluminal coronary angioplasty (PTCA) and cell lysis and serve as anticoagulants in extracorporeal recirculation (heartlung apparatus, haemodialysis). They can also serve in a general way for the prophylaxis of thrombosis, for example, after surgical operations.

Inhibitors of thrombin are suitable for the therapy and prophylaxis of
diseases whose pathogenetic mechanism is based, directly or indirectly, on the proteolytic action of thrombin,
diseases whose pathogenetic mechanism is based on the thrombin-dependent activation of receptors and signal transductions,
diseases accompanying the stimulation or inhibition of gene expressions in somatic cells,
diseases due to the mitogenetic action of thrombin,
diseases caused by a thrombin-dependent change in contractility and permeability of epithel cells,
thrombin-dependent thrombo-embolic events,
disseminated intravascular coagulation (DIC),
re-occlusion, and for shortening the reperfusion time in cases of co-medication with thrombolytics,
early re-occlusion and later restenosization following PTCA,—thrombin-induced proliferation of smooth muscle cells,—the accumulation of active thrombin in the CNS,
tumor growth, and to counteract adhesion and carcinosis of tumor cells.

A number of thrombin inhibitors of the D-Phe-Pro-Arg type is known for which good thrombin inhibition in vitro has been described: WO 9702284-A, WO 9429336-A1, WO 9857932-A1, WO 9929664-A1, U.S. Pat. No. 5,939,392-A, WO 200035869-A1, WO 200042059-A1, DE 4421052-A1, DE 4443390-A1, DE 19506610-A1, WO 9625426-A1, DE 19504504-A1, DE 19632772-A1, DE 19632773-A1, WO 9937611-A1, WO 9937668-A1, WO 9523609-A1, U.S. Pat. No. 5,705,487-1, WO 9749404-A1, EP-669317-A1, WO 9705108-A1, EP 0672658. However, some of this compounds exhibit low oral activity.

In WO 9965934 and Bioorg. Med. Chem. Lett., 9(14), 2013-2018, 1999, benzamidine derivatives of the NAPAP type are described which are coupled through a long spacer to pentasaccharides and thus show a dual antithrombotic principle of action. However, no oral activity of these compounds is described.

Activation of the complement system ultimately leads, through a cascade of ca 30 proteins, inter alia, to lysis of cells. Simultaneously, molecules are liberated which, like C5a, can lead to an inflammatory reaction. Under physiological conditions, the complement system provides a defence mechanism against foreign bodies, such as viruses, fungi, bacteria, or cancer cells. Activation by various routes takes place initially via proteases. By activation, these proteases are made capable of activating other molecules of the complement system, which may in turn be inactive proteases. Under physiological conditions, this system, like blood coagulation, is under the control of regulatory proteins, which counteract exuberant activation of the complement system. In such cases it is not advantageous to take measures to inhibit the complement system.

In some cases the complement system overreacts, however, and thus contributes to the pathologic physiology of diseases. In such cases, therapeutic action on the complement system causing inhibition or modulation of the exuberant reaction is desirable. Inhibition of the complement system is possible at various levels in the complement system by inhibition of various effectors. The literature provides examples of the inhibition of serine proteases at the C1 level with the aid of the C1 esterase inhibitor as well as inhibition at the level of C3 or C5 convertases by means of soluble complement receptor CR1 (sCR1), inhibition at the level of C5 by means of antibodies, and inhibition at the level of C5a by means of antibodies or antagonists. The tools used for achieving inhibition in the above examples are proteins. In the present invention, low-molecular substances are described which are used for inhibition of the complement system.

For such inhibition of the complement system some proteases utilizing various activation routes are particularly suitable. Of the class of thrombin-like serine proteases, such proteases are the complement proteases C1r and C1s for the classical route, factor D and factor B for the alternative route, and also MASP I and MASP II for the MBL route. The inhibition of these proteases then leads to a re-establishment of the physiological control of the complement system in the above diseases or pathophysiological states.

Generally speaking, all inflammatory disorders accompanied by the immigration of neutrophilic blood cells must be expected to involve activation of the complement system. Thus it is expected that with all of these disorders an improvement in the pathophysiological state will be achieved by causing inhibition of parts of the complement system.

The activation of complement is associated with the following diseases or pathophysiological states:
reperfusion syndrome following ischaemia; ischemic states occur during, say, operations involving the use of heartlung apparatus; operations in which blood vessels are generally compressed to avoid severe haemorrhage; myocardial infarction; thrombo-embolic cerebral infarct; pulmonary thrombosis, etc.;
hyper-acute rejection of an organ; specifically in the case of xenotransplantations;
failure of an organ, for example multiple failure of an organ or ARDS (adult respiratory distress syndrome);
diseases caused by injuries (skull injuries) or multiple injuries, such as thermal injuries (burns), and anaphylactic shock;
sepsis; “vascular leak syndrome”: with sepsis and following treatment with biological agents, such as interleukin 2, or following transplantation;
Alzheimer's disease and also other inflammatory neurological diseases such as Myastenia graevis, multiple sclerosis, cerebral lupus, Guillain Barrè syndrome; forms of meningitis; forms of encephalitis;
systemic Lupus erythematosus (SLE);
rheumatoid arthritis and other inflammatory diseases in the rheumatoid disease cycle, such as Behcet's syndrome; juvenile rheumatoid arthritis;
renal inflammation of various geneses, such as glomerular nephritis, or Lupus nephriti;
pancreatitis;
asthma; chronic bronchitis;
complications arising in dialysis for renal insufficiency; vasculitis; thyroiditis;
ulcerative colitis and also other inflammable disorders of the gastro-intestinal tract;
auto-immune disorders.
inhibition of the complement system; for example, the use of the C1s inhibitors of the invention can alleviate the side effects of pharmaceutical preparations based on activation of the complement system and reduce resultant hypersensitivity reactions.

Accordingly, treatment of the above mentioned diseases or pathophysiological states with complement inhibitors is desirable, particularly treatment with low-molecular inhibitors.

PUT and FUT derivatives are amidinophenol esters and amidinonaphthol esters respectively and have been described as complement inhibitors (eg, Immunology (1983), 49(4), 685-91).

Inhibitors are desired which inhibit C1s and/or C1r, but not factor D. Preferably, there should be no inhibition of lysis enzymes such as t-PA and plasmin.

Special preference is given to substances which effectively inhibit thrombin or C1s and C1r.

### PHARMACOLOGICAL EXAMPLES

### Example A

### Thrombin Time

Reagents: thrombin reagent (List No. 126,594, Boehringer, Mannheim, Germany)

### Preparation of Citrate Plasm:

9 parts of venous human blood from the V. cephalica are mixed with 1 part of sodium citrate solution (0.11 mol/L), followed by centrifugation. The plasma can be stored at −20° C.

### Experimental Method:

50 μl of the solution of the test probe and 50 μl of citrate plasma are incubated for 2 minutes at 37° C. (CL8, ball type, Bender & Hobein, Munich, FRG). Then 100 μl of thrombin reagent (37° C.) are added. The time taken for the fibrin clot to form is determined. The EC₁₀₀ values give the concentration at which the thrombin time is doubled.

### Example B

### Chromogenic Test for Thrombin Inhibitors

Reagents: human plasma thrombin (No. T 8885, Sigma, Deisenhofen, Germany)
substrate: H-D-Phe-Pip-Arg-pNA2HCl (S-2238, Chromogenix, Mölndahl, Sweden)
buffer: Tris 50 mmol/L, NaCl 154 mmol/L, pH 8.0

### Experimental Procedure:

The chromogenic test can be carried out in microtitration plates. 10 μl of the solution of substance in dimethyl sulfoxide are added to 250 μl of buffer containing thrombin (final concentration 0.1 NIH units/mL) and incubated over a period of 5 minutes at from 20° to 28° C. The test is initiated by the addition of 50 μL of substrate solution in buffer (final concentration 100 μmol/L), the mixture being incubated at 28° C., and, following a period of 5 minutes, the test is stopped by the addition of 50 μL of citric acid (35%). The absorption is measured at 405/630 nm.

### Example C

### Platelet Aggregation in the Platelet-Enriched Plasma

Reagents: human plasma thrombin (No. T-8885, Sigma, Deisenhofen, Germany)

### Production of the Citrate-Enriched Platelet-Enriched Plasm:

Venous blood from the Vena cephalica of healthy drug-free test persons is collected. The blood is mixed 9:1 with 0.13M trisodium citrate.
Platelet-enriched plasma (PRP) is produced by centrifugation at 250×g (for 10 minutes at room temperature). Platelet-impoverished plasma (PPP) is produced by centrifugation for 20 minutes at 3600×g. PRP and PPP can be kept in sealed PE vessels for a period of 3 hours at room temperature. The platelet concentration is measured with a cytometer and should be from 2.5 to 2.8·10⁻⁸/mL.

### Experimental Method:

The platelet aggregation is measured by turbitrimetric titration at 37° C. (PAP 4, Biodata Corporation, Horsham, Pa., USA). Before thrombin is added, 215.6 μL of PRP are incubated for 3 minutes with 2.2 μL of test probe and then stirred over a period of 2 minutes at 1000 rpm. At a final concentration of 0.15 NIH units/mL, 2.2 μL of thrombin solution produce the maximum aggregation effect at 37° C./1000 rpm. The inhibited effect of the test probes is determined by comparing the rate (rise) of aggregation of thrombin without test substance with the rate of aggregation of thrombin with test substance at various concentrations.

### Example D

### Color Substrate Test for C1r Inhibition

Reagents: C1r from human plasma, activated, two-chain (dual-chain) form (purity: ca 95% according to SDS gel). No foreign protease activity could be detected.
   substrate: Cbz-Gly-Arg-S-Bzl, Product No. WBAS012, (Polypeptide, D38304 Wolfenbüttel, Germany).
   color reagent: DTNB (5.5′-dinitro-bis(2-nitrobenzoic acid)) (No. 43,760, Fluka, CH 9470 Buchs, Switzerland).
   buffer: 150 mM Tris/HCl, pH 7.50

### Test Procedure:

The color substrate test for determining the C1s activity is carried out in 96-well microtitration plates.
10 μL of inhibitor solution in 20% strength dimethyl sulfoxide (dimethyl sulfoxide diluted with 15 mM Tris/HCl, pH 7.50) are added to 140 μL of test buffer containing C1s in a final concentration of 0.013 U/mL and DTNB in a final concentration of 0.27 mM/L. Incubation was carried out over a period of 10 minutes at from 20° to 25° C.
The test is started by the addition of 50 μL of a 1.5 mM substrate solution in 30% strength dimethyl sulfoxide (final concentration 0.375 mM/L). Following an incubation period of 30 minutes at from 20° to 25° C., the absorbance of each well at 405 nm is measured in a double-beam microtitrimetric plate photometer against a blank reading (without enzyme).

### Measuring Criterion:

IC₅₀: inhibitor concentration required in order to reduce the amidolytic C1r activity to 50%.

### Statistical Results:

Calculation is based on the absorbance as a function of inhibitor concentration.

### Example E

### Material and Methods

### Color Substrate Test for C1s Inhibition

Reagents: C1s from human plasm, activated, two-chain (dual-chain) form (purity: ca 95% according to SDS gel). No foreign protease activity could be detected.
   Substrate: Cbz-Gly-Arg-S-Bzl, Product No. WBAS012, (PolyPeptide, D38304 Wolfenbüttel, Germany)
   Color reagent: DTNB (5.5′-dinitro-bis(2-nitrobenzoic acid)) (No. 43,760, Fluka, CH 9470 Buchs, Switzerland) buffer: 150 mM Tris/HCl, pH 7.50

### Test Procedure:

The color substrate test for determining the C1s activity is carried out in 96-well microtitration plates.
10 μL of the inhibitor solution in 20% strength dimethyl sulfoxide (dimethyl sulfoxide diluted with 15 mM Tris/HCl, pH 7.50) are added to 140 μL of test buffer containing C1s in a final concentration of 0.013 U/mL and DTNB in a final concentration of 0.27 mM/L. Incubation is carried out over a period of 10 minutes at from 20° to 25° C. The test is started by the addition of 50 μL of a 1.5 mM substrate solution in 30% strength dimethyl sulfoxide (final concentration 0.375 mmol/L). Following an incubation period of 30 minutes at from 20° to 25° C., the absorbance of each well at 405 nm is measured in a double-beam microtitrimetric plate photometer against a blank reading (without enzyme).

### Measuring Criterion:

IC₅₀: inhibitor concentration required in order to reduce the amidolytic C1s activity to 50%.

### Statistical Results:

Calculation is based on the absorbance as a function of inhibitor concentration.

### Example F

### Confirmation of the Inhibition of Complement by the Classical Route Employing a Hemolytic Test

For measuring potential complement inhibitors use is made, in the manner of diagnostic tests, of a test for measuring the classical route (literature: Complement, A practical Approach; Oxford University Press; 1997; pp 20 et seq). The source of complement used for this purpose is human serum. A test of similar layout is, however, also carried out on various serums of other species in a similar manner. The indicating system used comprises erythrocytes of sheep. The antibody-dependent lysis of these cells and the thus exuded haemoglobin are a measure of the complement activity.

Reagents, Biochemical Products:

| | | |
|---|---|---|
| Veronal | Merck | #2760500 |
| Na-Veronal | Merck | #500538 |
| NaCl | Merck | #1.06404 |
| MgCl₂ × 6H₂O | Baker | #0162 |
| CaCl₂ × 6H₂O | Riedel de Haen | #31307 |
| Gelatin | Merck | #1.04078.0500 |
| EDTA | Roth | #8043.2 |
| Alsevers soln. | Gibco | #15190-044 |
| Penicillin | Gruenenthal | #P1507 10 mega |
| Ambozeptor | Behring | #ORLC |

Stock Solutions:
VBS stock solution: 2.875 g/L Veronal; 1.875 g/L Na-Veronal;
   42.5 g/L NaCl
Ca/Mg stock solution: 0.15 M Ca++, 1 M Mg++
EDTA stock solution: 0.1 M, pH 7.5

Buffer:
GVBS buffer: VBS stock solution diluted 1:5 with Finn Aqua;
   1 g/L of gelatin dissolved in some buffer at elevated temperature
GVBS++ buffer: Ca/Mg stock solution diluted 1:1000 in GVBS buffer
GVBS/EDTA buffer: EDTA stock solution diluted 1:10 in GVBS buffer

### Biogenic Components:

Sheep erythrocytes (SRBC): the blood of a wether was mixed 1:1 (v/v) with Alsevers solution and filtered through glass wool. There was added 1/10 volume of EDTA stock solution and 1 spatula tip of penicillin. Human serum: after centrifuging off the clotted portions at 4° C., the supernatant liquor was stored in aliquot portions at −70° C. All of the measurements were carried out on one batch. No essential deviations from serum of other test objects were found.

### Procedure:

### 1. Sensitization of the Erythrocytes:

SRBC's were washed three times with GVBS buffer. The number of cells was then adjusted to 5.00E+08 cells/mL in GVBS/EDTA buffer. Ambozeptor was added in a dilution of 1:600 and the SRBC's were then sensitized with antibody by incubation for 30 min at 37° C. with agitation. The cells were then washed three times with GVBS buffer at 4° C., then absorbed in GVBS++ buffer and adjusted to a cell count of 5×10⁸.

### 2. Lysis Batch:

Inhibitors were pre-incubated in GVBS++ for 10 min at 37° C. in a volume of 100 μL in various concentrations with human serum or serum of other species in suitable dilutions (for example 1:80 for human-serum; a suitable dilution is one at which ca 80% of the maximum cell lysis attainable with serum is achieved). 50 μL of sensitized SRBC's in GVBS++ were then added. Following incubation for one hour at 37° C. with agitation, the SRBC's were removed by centrifugation (5 minutes, 2500 rpm, 4° C.). 130 μL of the cell-free supernatant were transferred to a 96-well plate. The results were gained by measuring at 540 nm against GVBS++ buffer.

Evaluation was based on the absorption values at 540 nm.
(1): background; cells without serum
(3): 100% cell lysis; cells with serum
(x): readings on test probes

### Calculation:

$$\% cell lysis = \frac{( x ) - ( 1 ) \times 100 \%}{( 3 ) - ( 1 )}$$

### Example G

### Inhibitors Tested for Inhibition of Protease Factor D

Factor D plays a central role in the alternative route of the complement system. By reason of the low plasma concentration of factor D, the enzymatic step of cleavage of factor B by factor D represents the rate-limiting step in the alternative way of achieving complement activation. On account of the limiting role played by this enzyme in the alternative route, factor D is a target for the inhibition of the complement system.

The commercial substrate Z-Lys-S-Bzl * HCl is converted by the enzyme factor D (literature: C. M. Kam et al, J. Biol. Chem. 262 3444-3451, 1987). Detection of the cleaved substrate is effected by reaction with Ellmann's reagent. The resulting product is detected spectrophotometrically. The reaction can be monitored on-line. This makes it possible to take enzyme-kinetic readings.

### Material:

Chemicals:

| | | |
|---|---|---|
| Factor D | Calbiochem | 341273 |
| Ellmann's Reagent | Sigma | D 8130 |
| Z-Lys-S-Bzl * HCl (=substrate) | Bachem | M 1300 |
| | 50 mg/mL | |
| | (MeOH) | |
| NaCl | Riedel De Häen | 13423 |
| Triton-X-100 | Aldrich | 23,472-9 |
| Tris(hydroxymethyl)aminomethane | Merck | |
| Dimethylformamide (DMF) | | |

Buffer:

| | |
|---|---|
| 50 mM | Tris |
| 150 mM | NaCl |
| 0.01% | triton - X - 100 |
| pH 7.6 | |

Stock Solutions:

| | |
|---|---|
| Substrate | 20 mM (8.46 mg/mL = 16.92 μL (50 mg/mL) + |
| | 83.1 μL H₂O) |
| Ellmann's Reagent | 10 mM (3.963 mg/mL) in DMF |
| Factor D | 0.1 mg/mL |
| Samples (inhibitors) | 10⁻²M DMSO |

Procedure:

Batches:
Blank reading: 140 μL of buffer+4.5 μL of substrate (0.6 mM)+4.5 μL of Ellmann's reagent (0.3 mM)
Positive control: 140 μL of buffer+4.5 g/L, of substrate (0.6 mM)+4.5 μL of Ellmann's reagent (0.3 mM)+5 μL of factor D
Sample readings: 140 μL of buffer+4.5 μL of substrate (0.6 mM)+4.5 μL of Ellmann's reagent (0.3 mM)+1.5 μL of sample (10⁻⁴ M)+5 μL of factor D
The batches are pipetted together into microtitration plates. After mixing the buffer, substrate and Ellmann's reagent (inhibitor when required), the enzyme reaction is initiated by the addition of 5 μL of factor D in each case. Incubation takes place at room temperature for 60 min.

Readings:
Readings are taken at 405 nm over a period of 1 hour at intervals of 3 minutes.

Evaluation:
The results are plotted as a graph. The change in absorption per minute (Delta OD per minute; rising) is relevant for the comparison of inhibitors, since Kᵢ value of inhibitors can be ascertained therefrom.

In this test, the serin protease inhibitor FUT-175; Futhan, Torii; Japan was co-used as effective inhibitor.

### Example H

Confirmation of the inhibition of complement by the alternative route was obtained using a hemolytic test (literature: Complement, A practical Approach; Oxford University Press; 1997, pp 20 et seq).

The test is carried out on the lines of clinical tests. The test can be modified by additional activation by means of, say, Zymosan or cobra venom factor.

### Material:

| EGTA | Boehringer | 1093053 |
|---|---|---|
| (ethylene-bis(oxyethylenenitrilo)tetracetic | Mannheim | |
| acid | | |
| MgCl₂•6 H₂O | Merck | 5833,0250 |
| NaCl | Merck | 1.06404.1000 |
| D-glucose | Cerestar | |
| Veronal | Merck | 2760500 |
| Na-Veronal | Merck | 500538 |
| VBS—stock solution (5x) | gelatin Veronal buffer PD | |
| | Dr. Kirschfink; University of | |
| | Heidelberg, Institute for | |
| | Immunology; | |
| Gelatin | Merck | 1.04078.0500 |
| Tris(hydroxymethyl)aminomethane | Merck | 1.08382.0100 |
| CaCl₂ | Merck | No. 2382 |

Human serum was either procured from various contractors (eg, Sigma) or obtained from test persons in the polyclinic department of BASF Süd.

Guinea pig's blood was extracted and diluted 2:8 in citrate solution. Several batches were used without apparent differences.

### Stock Solutions:

VBS stock solution: 2.875 g/L Veronal
   1.875 g/L Na-Veronal
   42.5 g/L NaCl
GVBS: VBS stock solution diluted 1:5 with water (Finn Aqua)
   0.1% gelatin added
   and heated until gelatin had dissolved
   and then cooled
100 mM EGTA: 38.04 mg EGTA diluted in 500 mL of Finn Aqua and slowly treated with 10 M NaOH to raise the pH to 7.5 until dissolved,
then made up to 1 L.
Saline: 0.9% NaCl in water (Finn Aqua)
GTB: 0.15 mM CaCl₂
   141 mM NaCl
   0.5 mM MgCl₂.6H₂O
   10 mM Tris
   0.1% gelatin
   pH 7.2-7.3

### Procedure:

1. Cell preparation:
   The erythrocytes in the guinea pig's blood were washed with GTB a number of times by centrifugation (5 minutes at 1000 rpm) until the supernatant liquor was clear. The cell count was adjusted to 2·10⁹ cells/mL.
2. Procedure: the individual batches were incubated with agitation over a period of 30 minutes at 37° C. The assay was then stopped with 480 μL of ice-cold saline (physical solution of common salt) and the cells were removed by centrifugation at 5000 rpm over a period of 5 minutes. 200 μL of the supernatant liquor were measured at 405 nm by transfer thereof to a microtitration plate and evaluation in a microtitration plate photometer.

### Pipetting Table (Quantities in μL)

| | | | 100% | Background + | Max. |
|---|---|---|---|---|---|
| | Background | 100 % | Lysis + | factor D | lysis |
| | (−serum) | Lysis | factor D | (−serum) | (water) |
| Cells | 20 | 20 | 20 | 20 | 20 |
| Serum | | 20 | 20 | | |
| Mg - EGTA | 480 | 480 | 480 | 480 | |
| Factor D | | | 0.5 μg | 0.5 μg | |
| Saline (to | 480 | 480 | 480 | 480 | |
| stop the test | | | | | |
| H₂O | | | | | 980 |

### Results:

Assessment was made using the OD values.
(1): background; cells without serum
(3): 100% cell lysis+factor D; cells with serum
(x): readings on test probes

### Calculation:

$$\% cell lysis = \frac{( x ) - ( 1 ) \times 100 \%}{( 3 ) - ( 1 )}$$

### Example I

### Pharmacokinetics and Clotting Parameters in Rats

The test probes are dissolved in isotonic salt solution just prior to administration to Sprague Dawley rats in an awake state. The administration doses are 1 ml/kg for intravenous Bolus injection into the cereal vein and 10 ml/kg for oral administration, which is carried out per pharyngeal tube. Withdrawals of blood are made, if not otherwise stated, one hour after oral administration of 21.5 mg·kg⁻¹ or intravenous administration of 1.0 mg·kg⁻¹ of the test probe or corresponding vehicle (for control). Five minutes before the withdrawal of blood, the animals are narcotized by i.p. administration of 25% strength urethane solution (dosage 1 g·kg⁻¹ i.p.) in physiological saline. The A. carotis is prepared and catheterized, and blood samples (2 mL) are taken in citrate tubules (1.5 parts of citrate plus 8.5 parts of blood). Directly after blood sampling, the ecarin clotting time (ECT) in whole blood is determined. Following preparation of the plasma by centrifugation, the plasma thrombin time and the activated partial thromboplastin time (APTT) are determined with the aid of a coagulometer.

### Clotting Parameters:

Ecarin clotting time (ECT): 100 μL of citrate blood are incubated for 2 min at 37° C. in a coagulometer (CL 8, ball type, Bender & Hobein, Munich, German Federal Republic). Following the addition of 100 μL of warmed (37° C.) ecarin reagent (Pentapharm), the time taken for a fibrin clot to form is determined.

Activated thromboplastin time (APTT): 50 μL of citrate plasma and 50 μL of PTT reagent (Pathrombin, Behring) are mixed and incubated for 2 min at 37° C. in a coagulometer (CL 8, ball type, Bender & Hobein, Munich, German Federal Republic). Following the addition of 50 μL of warmed (37° C.) calcium chloride, the time taken for a fibrin clot to form is determined.

Thrombin time (TT): 100 μL of citrate-treated plasma are incubated for 2 min at 37° C. in a coagulometer (CL 8, ball type, Bender & Hobein, Munich, German Federal Republic). Following the addition of 100 μL of warmed (37° C.) thrombin reagent (Boehringer Mannheim), the time taken for a fibrin clot to form is determined.

### Example J

### Pharmacokinetics and Clotting Parameters in Dogs

The test probes are dissolved in isotonic salt solution just prior to administration to half-breed dogs. The administration doses are 0.1 ml/kg for intravenous Bolus injection and 1 ml/kg for oral administration, which is carried out per pharyngeal tube. Samples of venous blood (2 mL) are taken in citrate tubules prior to and also 5, 10, 20, 30, 45, 60, 90, 120, 180, 240, 300, and 360 min (if required, 420 min, 480 min, and 24 H) after intravenous administration of 1.0 mg/kg or prior to and also 10, 20, 30, 60, 120, 180, 240, 300, 360, 480 min and 24 h after oral dosage of 4.64 mg/kg. Directly after blood sampling, the ecarin clotting time (ECT) in whole blood is determined. Following preparation of the plasma by centrifugation, the plasma thrombin time and the activated partial thromboplastin time (APTT) are determine with the aid of a coagulometer.

In addition, the anti-F-IIa activity (ATU/mL) and the concentration of the substance are determined by their anti-F-IIa activity in the plasma by means of chromogenic (S 2238) thrombin assay, calibration curves with r-hirudin and the test substance being used.

The plasma concentration of the test probe forms the basis of calculation of the pharmacokinetic parameters: time to maximum plasma concentration (T max), maximum plasma concentration; plasma half-life, t_{0.5}; area under curve (AUC); and resorbed portion of the test probe (F).

### Clotting Parameters:

Ecarin clotting time (ECT): 100 μL citrate-treated blood are incubated for 2 min at 37° C. in a coagulometer (CL 8, ball type, Bender & Hobein, Munich, German Federal Republic). Following the addition of 100 μL of warmed (37° C.) ecarin reagent (Pentapharm), the time taken for a fibrin clot to form is determined.
Activated thromboplastin time (APTT): 50 μL citrate-treated plasma and 50 μL of PTT reagent (Pathrombin, Behring) are mixed and incubated for 2 min at 37° C. in a coagulometer (CL 8, ball type, Bender & Hobein, Munich, German Federal Republic). Following the addition of 50 μL of warmed (37° C.) calcium chloride, the time taken for a fibrin clot to form is determined.
Thrombin time (TT): 100 μL of citrate-treated plasma is incubated for 2 min at 37° C. in a coagulometer (CL 8, ball type, Bender & Hobein, Munich, German Federal Republic). Following the addition of 100 μL of warmed (37° C.) thrombin reagent (Boehringer Mannheim), the time taken for a fibrin clot to form is determined.

The present invention relates to peptide substances and peptidomimetic substances, to the preparation thereof, and to the use thereof as thrombin inhibitors or complement inhibitors. In particular, the substances concerned are those having an amidine group as terminal group on the one hand and a polyhydroxyalkyl or polyhydroxcycloalkyl group—which can comprise several units—as the second terminal group on the other hand.

The invention relates to the use of these novel substances for the production of thrombin inhibitors, complement inhibitors, and, specifically, inhibitors of C1s and C1r.

In particular, the invention relates to the use of chemically stable substances of the general formula I, to their tautomers and pharmacologically compatible salts and prodrugs for the production of medicinal drugs for the treatment and prophylaxis of diseases which can be alleviated or cured by partial or complete inhibition, particularly selective inhibition, of thrombin or C1s and/or C1r.

Formula I has the general structure

A-B-D-E-G-K-L (I),

in which
A stands for H, CH₃, H—(R^{A1})i^{A}
in which
R^{A1} denotes

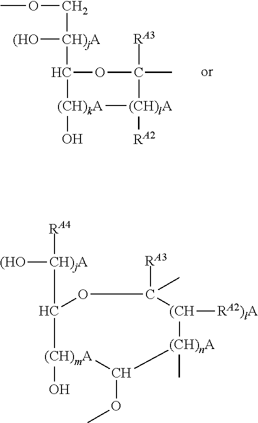

in which R^{A2} denotes H, NH₂, NH—COCH₃, F, or NHCHO,
   R^{A3} denotes H or CH₂OH,
   R^{A4} denotes H, CH₃, or COOH,
   ᵢA is 1 to 20,
   ⱼA is 0, 1, or 2,
   ₖA is 2 or 3,
   ₗA is 0 or 1,
   ₘA is 0, 1, or 2,
   ₙA is 0, 1, or 2,
the groups R^{A1} being the same or different when ᵢA is greater than 1;
B denotes

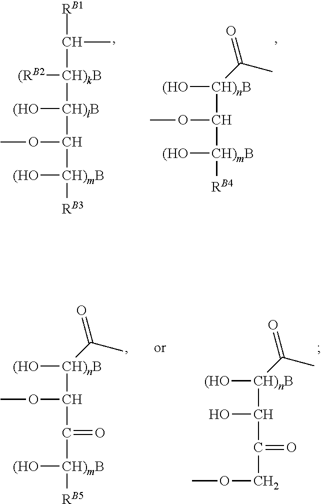

A-B can stand for

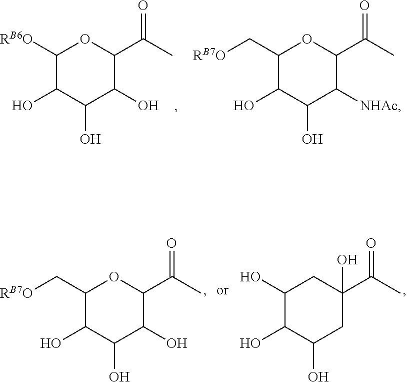

or for a neuraminic acid radical or N-acetylneuraminic acid radical bonded through the carboxyl function,
in which
R^{B1} denotes H, CH₂OH, or C₁₋₄ alkyl,
R^{B2} denotes H, NH₂, NH—COCH₃, F, or NHCHO,
R^{B3} denotes H, C₁₋₄ alkyl, CH₂—O—(C₁₋₄ alkyl), COOH, F, NH—COCH₃,
   or
      CONH₂,
R^{B4} denotes H, C₁₋₄ alkyl, CH₂—O—(C₁₋₄ alkyl), COOH, or CHO, in which latter case intramolecular acetal formation may take place,
R^{B5} denotes H, C₁₋₄ alkyl, CH₂—O—(C₁₋₄ alkyl), or COOH,
ₖB is 0 or 1,
ₗB is 0, 1, 2, or 3 (ₗB≠0 when A=R^{B1}=R^{B3}=H, ₘB=ₖB=0 and D is a bond),
ₘB is 0, 1, 2, 3, or 4,
ₙB is 0, 1, 2, or 3,
R^{B6} denotes C₁₋₄ alkyl, phenyl, or benzyl, and
R^{B7} denotes H, C₁₋₄ alkyl, phenyl, or benzyl;
D stands for a bond or for

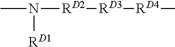

in which
R^{D1} denotes H or C₁₋₄ alkyl,
R^{D2} denotes a bond or C₁₋₄ alkyl,
R^{D3} denotes

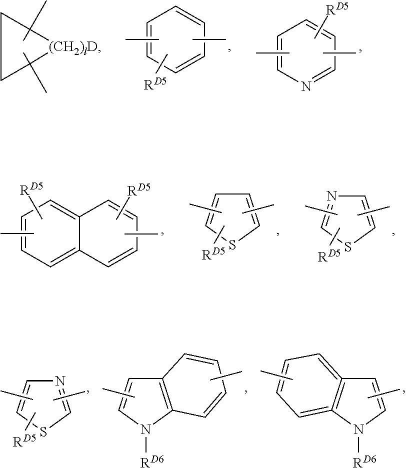

in which
   ₗD is 1, 2, 3, 4, 5, or 6,
   R^{D5} denotes H, C₁₋₄ alkyl, or Cl, and
   R^{D6} denotes H or CH₃,
and in which a further aromatic or aliphatic ring can be condensed onto the ring systems defined for R^{D3}, and
   R^{D4} denotes a bond, C₁₋₄ alkyl, CO, SO₂, or —CH₂—CO;
      E stands for

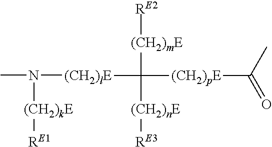

in which
ₖE is 0, 1, or 2,
ₗE is 0, 1, or 2,
ₘE is 0, 1, 2, or 3,
ₙE is 0, 1, or 2,
ₚE is 0, 1, or 2,
R^{E1} denotes H, C₁₋₆ alkyl, C₃₋₈ cycloalkyl, aryl (particularly phenyl or naphthyl), heteroaryl (particularly pyridyl, thienyl, imidazolyl, or indolyl), and C₃₋₈ cycloalkyl having a phenyl ring condensed thereto, which groups may carry up to three identical or different substituents selected from the group consisting of C₁₋₆ alkyl, OH, O—(C₁₋₆ alkyl), F, Cl, and Br,
R^{E1} may also denote R^{E4}OCO—CH₂— (where R^{E4} denotes H, C₁₋₁₂ alkyl, or C₁₋₃ alkylaryl),
R^{E2} denotes H, C₁₋₆ alkyl, C₃₋₈ cycloalkyl, aryl (particularly phenyl or naphthyl), heteroaryl (particularly pyridyl, furyl, thienyl, imidazolyl, or indolyl), tetrahydropyranyl, tetrahydrothiopyranyl, diphenylmethyl, and dicyclohexylmethyl, C₃₋₈ cycloalkyl having a phenyl ring condensed thereto, which groups may carry up to three identical or different substituents selected from the group consisting of C₁₋₆ alkyl, OH, O—(C₁₋₆ alkyl), F, Cl, and Br, and may also denote CH(CH₃)OH or CH(CF₃)₂,
R^{E3} denotes H, C₁₋₆ alkyl, C₃₋₈ cycloalkyl, aryl (particularly phenyl or naphthyl), heteroaryl (particularly pyridyl, theinyl, imidazolyl, or indolyl), and C₃₋₈ cycloalkyl having a phenyl ring condensed thereto, which groups may carry up to three identical or different substituents selected from the group consisting of C₁₋₆ alkyl, OH, O—(C₁₋₆ alkyl), F, Cl, and Br,
   the groups defined for R^{E1} and R^{E2} may be interconnected through a bond, and the groups defined for R^{E2} and R^{E3} may also be interconnected through a bond,
R^{E2} may also denote COR^{E5} (where R^{E5} denotes OH, O—(C₁₋₆ alkyl), or O—(C₁₋₃ alkylaryl)), CONR^{E6}R^{E7} (where R^{E6} and R^{E7} denote H, C₁₋₆ alkyl, or C₀₋₃ alkylaryl), or NR^{E6}R^{E7},
   E may also stand for D-Asp, D-Glu, D-Lys, D-Orn, D-His, D-Dab, D-Dap, or D-Arg;
   G stands for

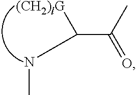

where ₗG is 2, 3, 4, or 5, and one of the CH₂ groups in the ring is replaceable by O, S, NH, N(C₁₋₃ alkyl), CHOH, CHO(C₁₋₃ alkyl), C(C₁₋₃ alkyl)₂, CH(C₁₋₃ alkyl), CHF, CHCl, or CF₂,

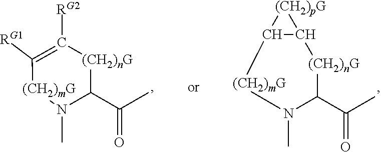

in which
ₘG is 0, 1, or 2,
ₙG is 0, 1, or 2,
ₚG is 0, 1, 2, 3, or 4,
R^{G1} denotes H, C₁₋₆ alkyl, or aryl,
R^{G2} denotes H, C₁₋₆ alkyl, or aryl,
and R^{G1} and R^{G2} may together form a —CH═CH—CH═CH— chain,
   G may also stand for

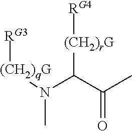

in which
_{q}G is 0, 1, or 2,
ᵣG is 0, 1, or 2,
R^{G3} denotes H, C₁₋₆ alkyl, C₃₋₈ cycloalkyl, or aryl,
R^{G4} denotes H, C₁₋₆ alkyl, C₃₋₈ cycloalkyl, or aryl (particularly phenyl or naphthyl);
   K stands for

NH—(CH₂)ₙK-Q^{K}
in which
ₙK is 0, 1, 2, or 3,
Q^{K} denotes C₂₋₆ alkyl, whilst up to two CH₂ groups may be replaced by O or S,
Q^{K} also denotes

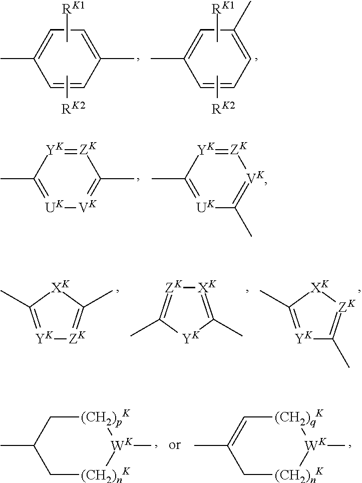

in which
R^{K1} denotes H, C₁₋₃ alkyl, OH, O—C(₁₋₃ alkyl), F, Cl, or Br,
R^{K2} denotes H, C₁₋₃ alkyl, O—(C₁₋₃ alkyl), F, Cl, or Br,
X^{K} denotes O, S, NH, N—(C₁₋₆ alkyl),
Y^{K} denotes ═CH—,

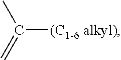

═N—, or

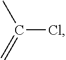

Z^{K} denotes ═CH—,

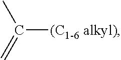

═N—, or

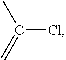

U^{K} denotes ═CH—,

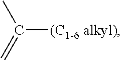

═N—, or

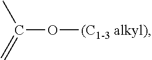

V^{K} denotes ═CH—,

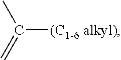

═N—, or

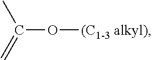

W^{K} denotes

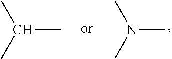

but in the latter case L may not be a guanidine group,
ₙK is 0, 1, or 2,
ₚK is 0, 1, or 2, and
_{q}K is 1 or 2;
   L stands for

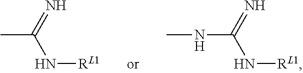

in which
R^{L1} denotes H, OH, O—(C₁₋₆ alkyl), O—(CH₂)₀₋₃-phenyl,
   CO—(C₁₋₆ alkyl), CO₂—(C₁₋₆ alkyl), or CO₂—(C₁₋₃ alkylaryl).

Preference is given to the following compounds of formula I

A-B-D-E-G-K-L (I),

in which
A stands for H or H—(R^{A1})i^{A}
in which
R^{A1} denotes

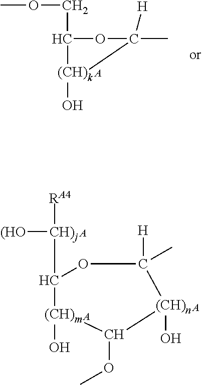

in which
   R^{A4} denotes H, CH₃, or COOH,
   ᵢA is 1 to 6,
   ⱼA is 0, 1, or 2,
   ₖA is 2 or 3,
   ₘA is 0, 1, or 2,
   ₙA is 0, 1, or 2,
the groups R^{A1} being the same or different when ᵢA is greater than 1;
   B denotes

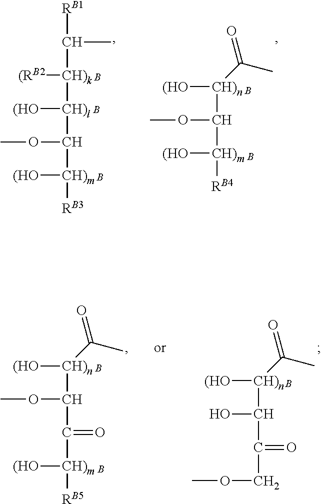

A-B stands for

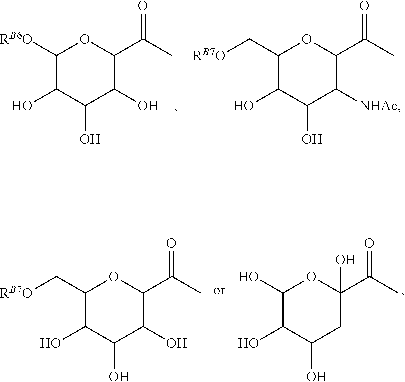

in which
R^{B1} denotes H or CH₂OH,
R^{B2} denotes H, NH₂, NH—COCH₃, or F,
R^{B3} denotes H, CH₃, CH₂—O—(C₁₋₄ alkyl), or COOH,
R^{B4} denotes H, C₁₋₄ alkyl, CH₂—O—(C₁₋₄ alkyl), COOH, or CHO, in which latter case intramolecular acetal formation may take place,
R^{B5} denotes H, CH₃, CH₂—O—(C₁₋₄ alkyl), or COOH,
ₖB is 0 or 1,
ₗB is 0, 1, 2, or 3 (ₗB≠0 when A=R^{B1}=R^{B3}=H, ₘB=ₖB=0, and D is a bond),
ₘB is 0, 1, 2, or 3,
ₙB is 0, 1, 2, or 3,
R^{B6} denotes C₁₋₄ alkyl, phenyl, or benzyl, and
R^{B7} denotes H, C₁₋₄ alkyl, phenyl, or benzyl;
   D stands for a bond or for

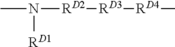

in which
   R^{D1} denotes H or C₁₋₄ alkyl,
R^{D2} denotes a bond or C₁₋₄ alkyl,
R^{D3} denotes

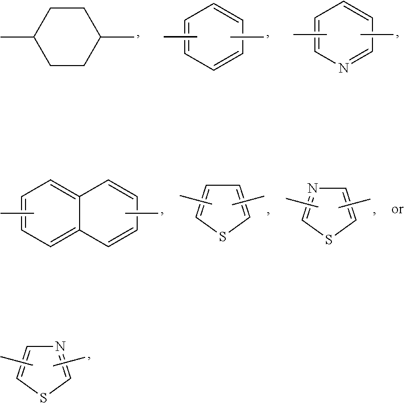

R^{D4} denotes a bond, C₁₋₄ alkyl, CO, SO₂, or —CH₂—CO;
   E stands for

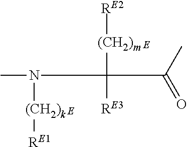

in which
ₖE is 0, 1, or 2,
ₘE is 0, 1, 2, or 3,
R^{E1} denotes H, C₁₋₆ alkyl, or C₃₋₈ cycloalkyl, which groups may carry up to three identical or different substituents selected from the group consisting of C₁₋₆ alkyl, OH, and O—(C₁₋₆ alkyl),
R^{E2} denotes H, C₁₋₆ alkyl, C₃₋₈ cycloalkyl, aryl (particularly phenyl or naphthyl), heteroaryl (particularly pyridyl, furyl, or thienyl), tetrahydropyranyl, diphenylmethyl, or dicyclohexylmethyl, which groups may carry up to three identical or different substituents selected from the group consisting of C₁₋₆ alkyl, OH, O—(C₁₋₆ alkyl), F, Cl, and Br, and may also denote CH(CF₃)₂;
R^{E3} denotes H, C₁₋₆ alkyl, or C₃₋₈ cycloalkyl, and
R^{E2} may also denote COR^{E5} (where R^{E5} denotes OH, O—C₁₋₆ alkyl, or O—(C₁₋₃ alkylaryl)), CONR^{E6}R^{E7} (where R^{E6} and R^{E7} each denote H, C₁₋₆ alkyl, or C₀₋₃ alkylaryl), or NR^{E6}R^{E7};
   E may also stand for D-Asp, D-Glu, D-Lys, D-Orn, D-His, D-Dab, D-Dap, or D-Arg;
   G stands for

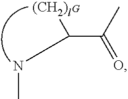

where ₗG is 2, 3, or 4, and one of the CH₂ groups in the ring is replaceable by O, S, NH, N(C₁₋₃ alkyl), CHOH, or CHO(C₁₋₃ alkyl);

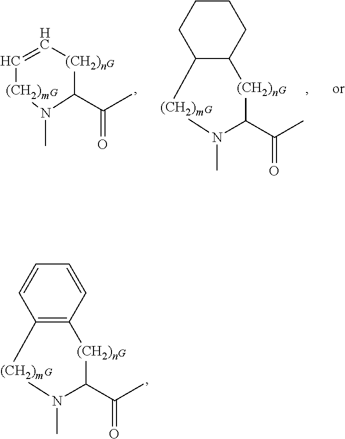

in which
   ₘG is 0, 1, or 2;
      ₙG is 0 or 1;
         K stands for

NH—(CH₂)ₙK-Q^{K}
in which
   ₙK C is 1 or 2,
   Q^{K} denotes

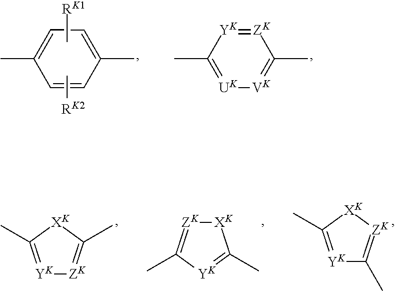

in which
R^{K1} denotes H, C₁₋₃ alkyl, OH, O—(C₁₋₃ alkyl), F, Cl, or Br,
R^{K2} denotes H, C₁₋₃ alkyl, O—(C₁₋₃ alkyl), F, Cl, or Br,
X^{K} denotes O, S, NH, N—(C₁₋₆ alkyl),
Y^{K} denotes ═CH—,

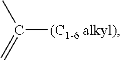

═N—, or

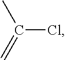

Z^{K} denotes ═CH—,

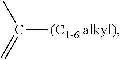

═N—, or

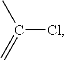

U^{K} denotes ═CH—,

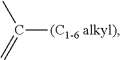

═N—, or

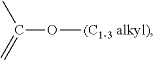

and
L stands for

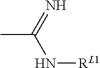

in which
R^{L1} denotes H, OH, O—(C₁₋₆ alkyl), or CO₂—(C₁ alkyl).

Preferred thrombin inhibitors are compounds of formula I

A-B-D-E-G-K-L (I),
in which
   A stands for H or H—(R^{A1})i^{A}
in which
R^{A1} denotes

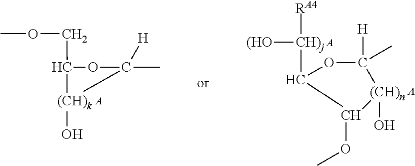

in which
   R^{A4} denotes H or COOH,
   ᵢA is 1 to 6,
   ⱼA is 0 or 1,
   ₖA is 2 or 3,
   ₙA is 1 or 2,
the groups R^{A1} being the same or different when ᵢA is greater than 1;
   B denotes

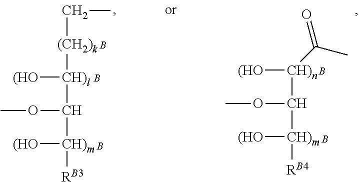

in which
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3, and
ₙB is 1, 2, or 3;
   D stands for a bond;
   E stands for

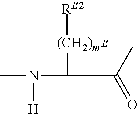

in which
ₘE is 0 or 1,
R^{E2} denotes H, C₁₋₆ alkyl, C₃₋₈ cycloalkyl, phenyl, diphenylmethyl, or dicyclohexylmethyl, which groups may carry up to three identical or different substituents selected from the group consisting of C₁₋₄ alkyl, OH, O—CH₃, F, and Cl;
   G stands for

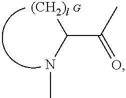

where ₗG is 2, 3, or 4 and one of the CH₂ groups in the ring is replaceable by O, S, NH, or N(C₁₋₃ alkyl),

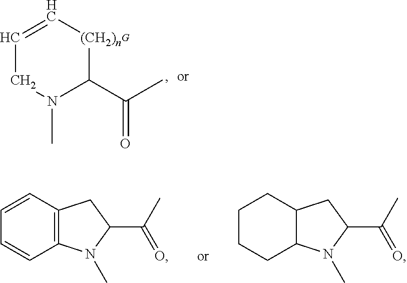

in which
ₙG is 0 or 1;
   K stands for

NH—CH₂-Q^{K}
in which
Q^{K} denotes

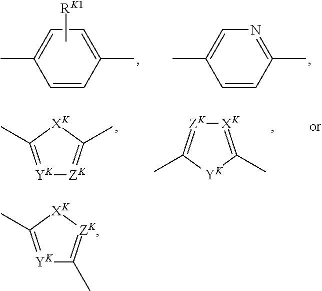

in which
R^{K1} denotes H, CH₃, OH, O—CH₃, F, or Cl,
X^{K} denotes O, S, NH, N—CH₃,
Y^{K} denotes ═CH—,

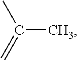

or ═N—,
Z^{K} denotes ═CH—,

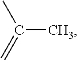

or ═N—,
   L stands for

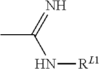

in which
R^{L1} denotes H, OH, or CO₂—(C₁₋₆ alkyl).

Preferred complement inhibitors are compounds of formula I

A-B-D-E-G-K-L (I),

in which
A stands for H or H—(R^{A1})i^{A}
in which
R^{A1} denotes

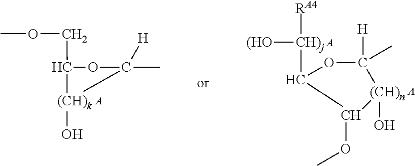

in which
   R^{A4} denotes H or COOH,
   ᵢA is 1 to 6,
   ⱼA is 0 or 1,
   ₖA is 2 or 3,
   ₙA is 1 or 2,
the groups R^{A1} being the same or different when ᵢA is greater than 1;
   B denotes

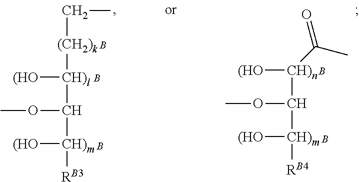

A-B stands for

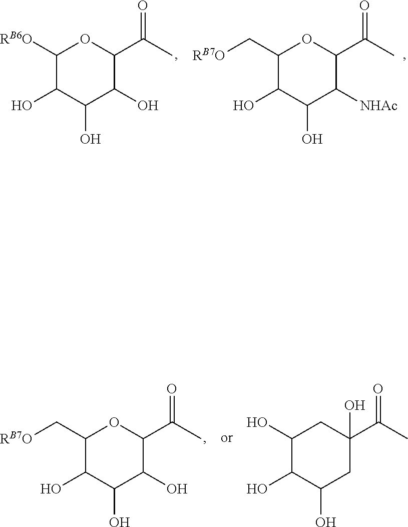

in which
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3,
ₙB is 1, 2, or 3,
R^{B6} denotes C₁₋₄ alkyl, phenyl, or benzyl, and
R^{B7} denotes H, C₁₋₄ alkyl, phenyl, or benzyl,
D stands for

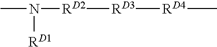

in which
   R^{D1} denotes H or C₁₋₄ alkyl,
   R^{D2} denotes a bond or C₁₋₄ alkyl,
   R^{D3} denotes

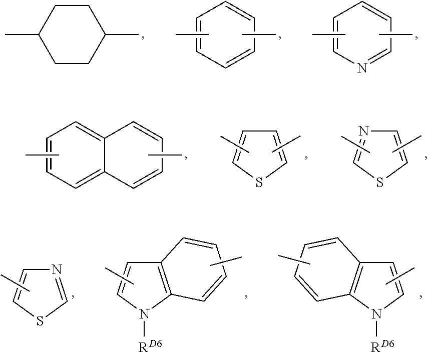

in which
R^{D4} denotes a bond, C₁₋₄ alkyl, CO, SO₂, or —CH₂—CO, and
R^{D6} denotes H or CH₃;
   E stands for

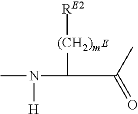

in which
ₘE is 0 or 1,
R^{E2} denotes H, C₁₋₆ alkyl, or C₃₋₈ cycloalkyl, which groups may carry up to three identical or different substituents selected from the group consisting of C₁₋₄ alkyl, OH, O—CH₃, F, and Cl;
   G stands for

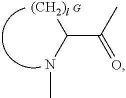

where ₗG is 2, 3, or 4 and one of the CH₂ groups in the ring is replaceable by O, S, NH, or N(C₁₋₃ alkyl),

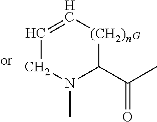

in which
ₙG is 0 or 1;
   K stands for

NH—CH₂-Q^{K}
in which
Q^{K} denotes

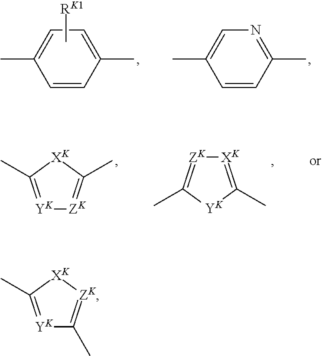

in which
R^{K1} denotes H, CH₃, OH, O—CH₃, F, or Cl,
X^{K} denotes O, S, NH, N—CH₃,
Y^{K} denotes ═CH—,

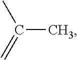

or ═N—,
Z^{K} denotes ═CH—,

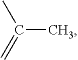

or ═N—; and
   L stands for

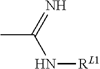

in which
R^{L1} denotes H, OH, or CO₂—(C₁₋₆ alkyl).

Particularly preferred thrombin inhibitors are compounds of formula I

A-B-D-E-G-K-L (I),

in which
A stands for H or H—(R^{A1})i^{A}
in which
R^{A1} denotes

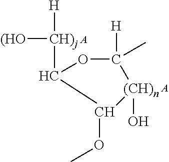

in which
   ᵢA is 1 to 6,
   ⱼA is 0 or 1,
   ₙA is 1 or 2,
the groups R^{A1} being the same or different when ᵢA is greater than 1;
   B denotes

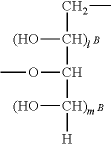

in which
ₗB is 1, 2, or 3,
ₘB is 1 or 2,
   D stands for a bond,
   E stands for

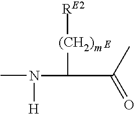

in which
ₘE is 0 or 1,
R^{E2} denotes H, C₁₋₆ alkyl, C₃₋₈ cycloalkyl, phenyl, diphenylmethyl, or dicyclohexylmethyl,
   building block E preferably exhibiting D configuration,
      G stands for

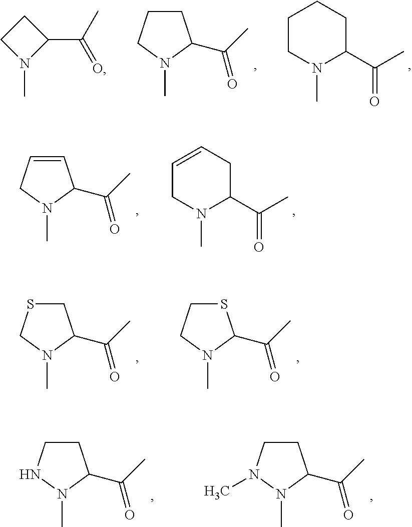

building block G preferably exhibiting L configuration;
   K stands for

NH—CH₂-Q^{K}
in which
Q^{K} denotes

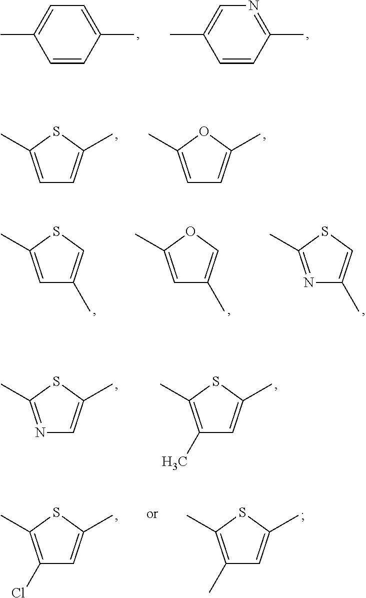

and
L stands for

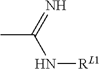

in which
R^{L1} denotes H, OH, or CO₂—(C₁₋₆ alkyl).

Particularly preferred complement inhibitors are compounds of formula I

A-B-D-E-G-K-L (I),

in which
A stands for H or H—(R^{A1})i^{A}
in which
R^{A1} denotes

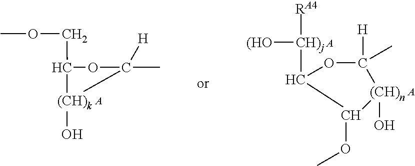

in which
   R^{A4} denotes H or COOH,
   ᵢA is 1 to 6,
   ⱼA is 0 or 1,
   ₖA is 2 or 3,
   ₙA is 1 or 2,
the groups R^{A1} being the same or different when ᵢA is greater than 1;
   B denotes

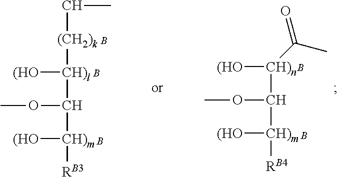

A-B stands for

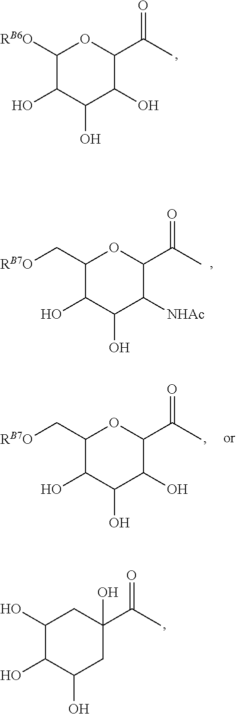

in which
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3,
ₙB is 1, 2, or 3,
R^{B6} denotes C₁₋₄ alkyl, phenyl, or benzyl, and
R^{B7} denotes H, C₁₋₄ alkyl, phenyl, or benzyl,
   D stands for

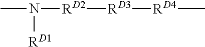

in which
   R^{D1} denotes H,
R^{D2} denotes a bond or C₁₋₄ alkyl,
R^{D3} denotes

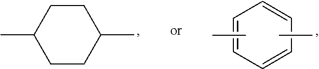

R^{D4} denotes a bond, C₁₋₄ alkyl, CO, SO₂, or —CH₂—CO, and
   E stands for

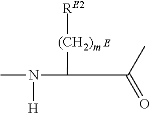

in which
ₘE is 0 or 1,
R^{E2} denotes H, C₁₋₆ alkyl, or C₃₋₈ cycloalkyl, which groups may carry up to three identical or different substituents selected from the group consisting of F and Cl;
   G stands for

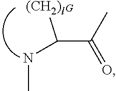

where ₗG is 2

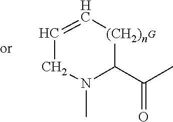

in which
   ₙG is 0,
      K stands for

NH—CH₂-Q^{K}
in which
Q^{K} denotes

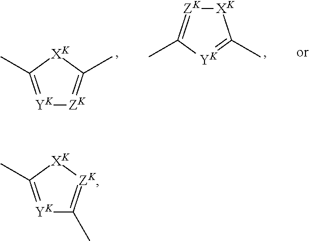

in which
X^{K} denotes S,
Y^{K} denotes ═CH—, or ═N—,
Z^{K} denotes ═CH—, or ═N—,
   and
   L stands for

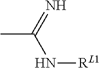

in which
R^{L1} denotes H or OH.

Preferred building blocks A-B are:

| | |
|---|---|
| D-Fructo | 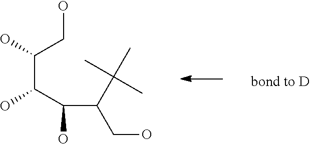 |
| D-Turano- | 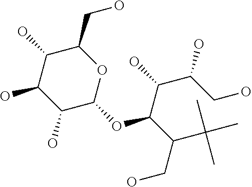 |
| 3-O-Methyl- D-glucopyrano- | 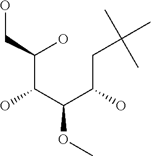 |
| D-Galacturo- | 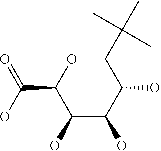 |
| Glucuronamo- | 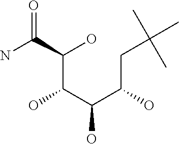 |
| N-Acetyl- neuraminic | 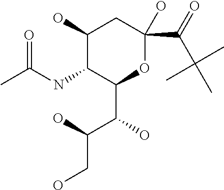 |
| D-Digitoxo | 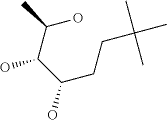 |
| Maltotrio- | 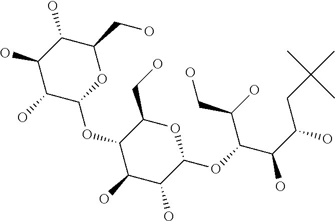 |
| Maltotetrao- | 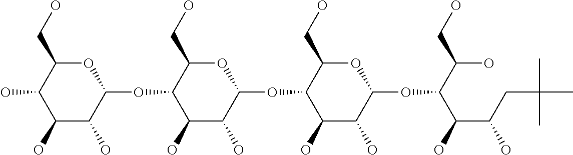 |
| 2-Deoxy-D- galacto | 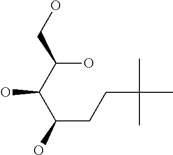 |
| 2-Acetamido- 2-deoxy- 3-O-(delta-d- galacto-pyrano- syl)-D-gluco- pyrano | 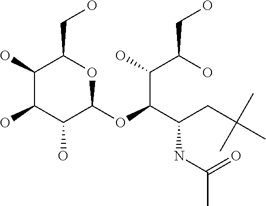 |
| D-Mannohep- tulo- | 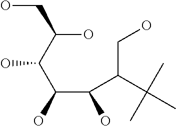 |
| alpha-Spphoro- | 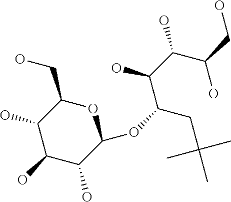 |
| N-Acetyl-D- Mannosami- | 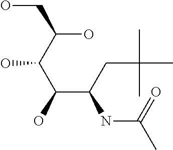 |
| 6-Acetamido-6- Deoxy-alpha- D-Glucopyrano- | 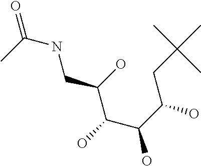 |
| 3-O-Beta-D- Galatopyranosyl- D-Arabino- | 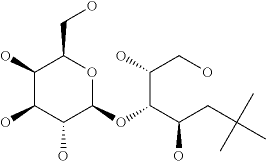 |
| D-Glucohepto- | 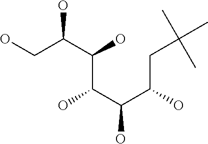 |
| Nigero- | 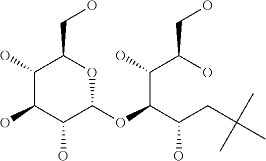 |
| D-Glucoheptulo- | 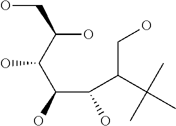 |
| Xylotrio- | 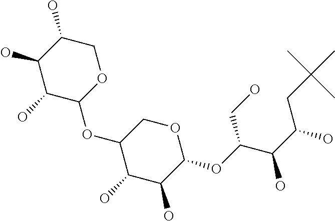 |
| 2-Acetamido-2- Deoxy-6-O-(beta- D-galactopyra- nosyl)-D-gluco- pyrano- | 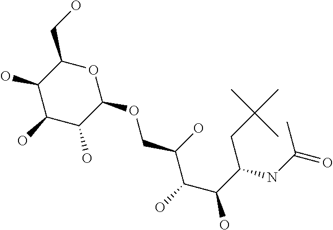 |
| 4-O-(4-O-[6-O- alpha-D-gluco- pyranosyl-alpha- glucopyranosyl]- alpha-D-gluco- pyr- | 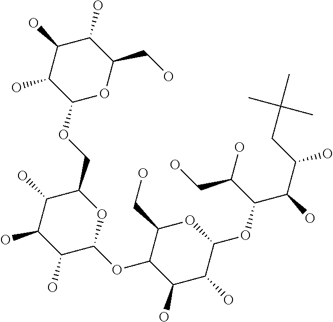 |
| 2-Acetamido-6- O-(2-acetamido- 2-deoxy-beta- D-glucopyrano- syl)-2-deoxy- D-glucopyran- | 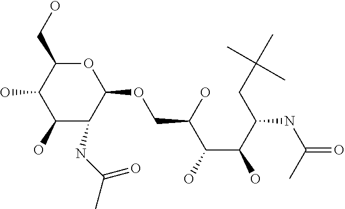 |
| 6-O-(2-Aceta- mido-2-deoxy- beta-D-glucopy- ranosyl)-D-galac- topyrano- | 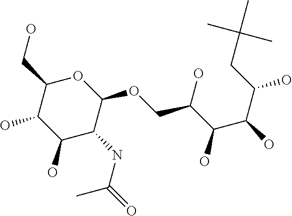 |
| 2-Acetamido-2- deoxy-4-O-([4-O- beta-D-galacto- pyranosyl]-beta- D-galacto- pyranosyl)- | 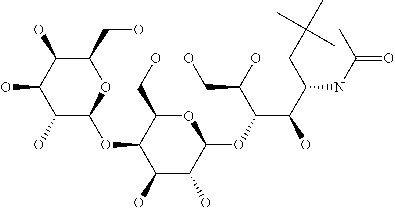 |
| N-Acetyl-D- glucosamin- | 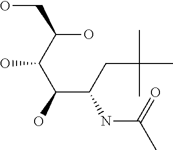 |
| 2-Fluoro-2-deoxy- D-galactopy- rano- | 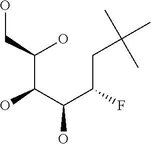 |
| 6-Deoxy- D-gluco- | 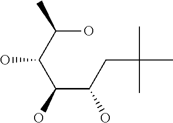 |
| L-Allo- | 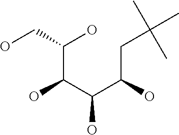 |
| 3-O-Methyl- gluco- | 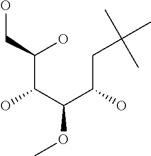 |
| D-Allo- | 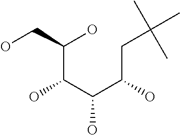 |
| 6-Fluoro-6-deoxy- D-galactopy- rano- | 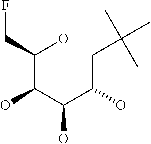 |
| D-Gluco- | 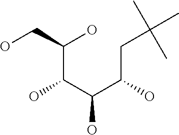 |
| Dextro- | 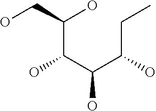 |
| N-Acetyl- lactosamin- | 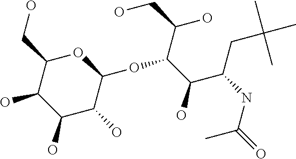 |
| L-Galacto- | 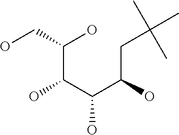 |
| L-Gluco- | 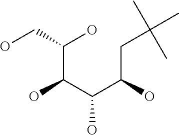 |
| 4-O-alpha- D-galactopyrano- syl-D-galacto- pyrano- | 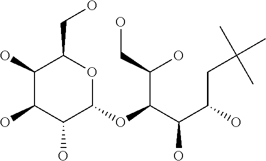 |
| 2-Acetamido- 2-deoxy-4-O([4- O-beta-D-galac- topyrano- syl]-beta-D-ga- lactopyranosyl)- | 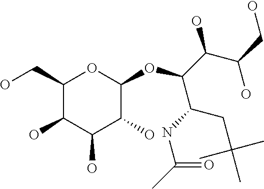 |
| 6-Fluoro-6-deoxy- D-glucopyrano- | 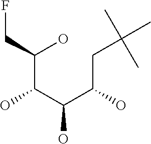 |
| L-Lyxo- | 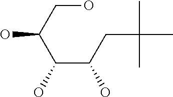 |
| L-Manno- | 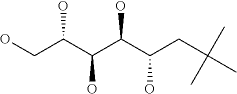 |
| D-Manno- | 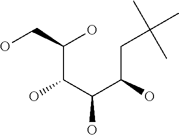 |
| N-Acetyl-D- glucosamin- | 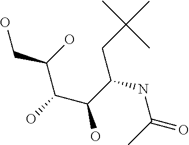 |
| D-Lyxo- | 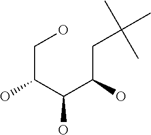 |
| D-Lacto- | 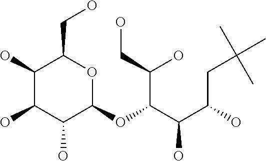 |
| Maltoheptao- | 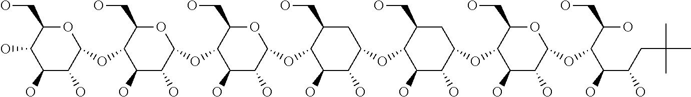 |
| D-Talo- | 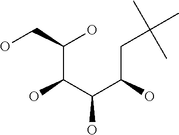 |
| L-Talo- | 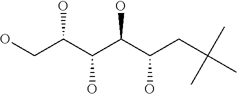 |
| Neohesperido- | 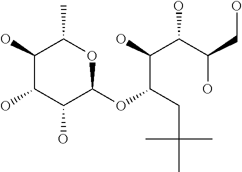 |
| N-Acetyl-D- galactosamin- | 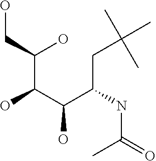 |
| Isomalto- | 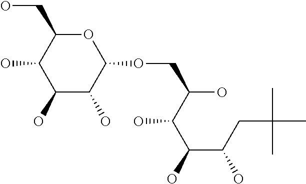 |
| Beta-Malto- | 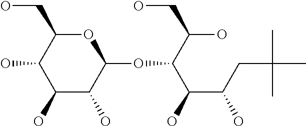 |
| L-Fructo- | 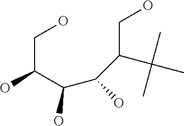 |
| 6-O-Methyl- D-galactopyrano- | 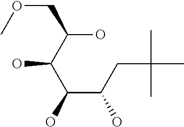 |
| 2-Deoxy- D-Ribo- hexopyrano- | 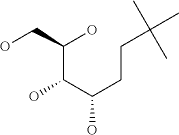 |
| Alpha-D- Kojibio- | 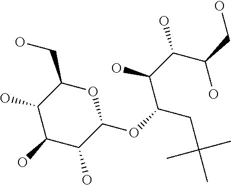 |
| 2-O-Methyl- D-xylo- | 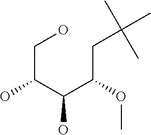 |
| L-Fluco- | 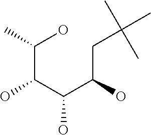 |
| 6-O-Beta-D- galactopyrano- syl- D-galacto- | 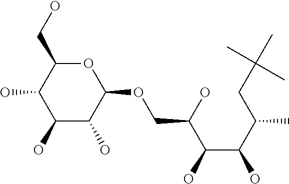 |
| L-Gulo- | 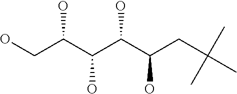 |
| D-Gulo- | 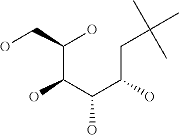 |
| D-Ido- | 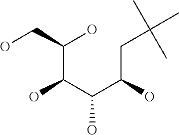 |
| L-Ido- | 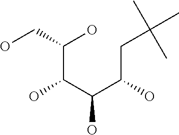 |
| (4-O-(4-O-Beta- D-galacto- pyranosyl)-beta- D-galacto- pyranosyl)- D-glucopyrano- | 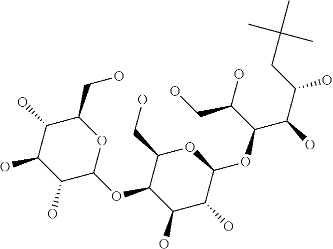 |
| D-Cellotrio- | 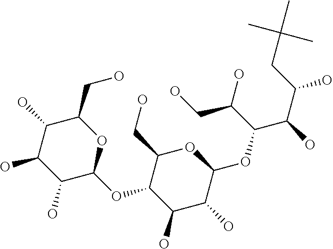 |
| Laminaribio- | 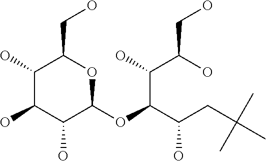 |
| 3-O-alpha- D-mannopyrano- syl-D-mannopy- rano- | 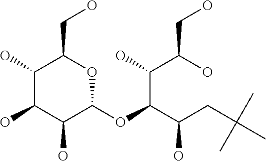 |
| 4-O-beta- Galacto- pyranosyl- D-mannopyrano- | 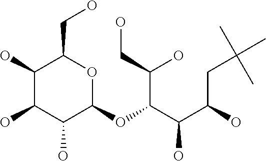 |
| Isomaltotrio- | 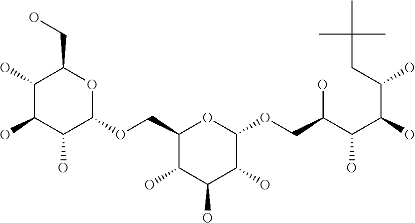 |
| D-Galacturonic- | 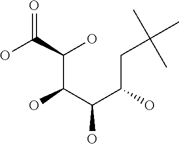 |
| L-Rhamno- | 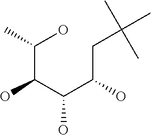 |
| D-Altro- | 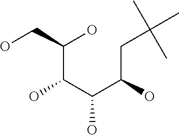 |
| N,N′-Diacetyl- chitobio- | 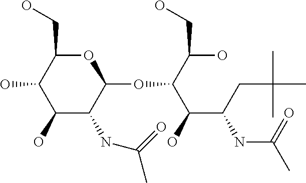 |
| D-Glucuronic- | 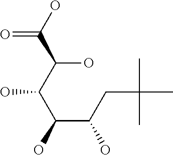 |
| (+)-Digitoxo- | 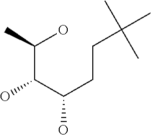 |
| 6-O-[2-Aceta- mido-2-deoxy- 4-O-(beta-D- galacto- pyranosyl)- beta-D-gluco- pyranosyl]-D- | 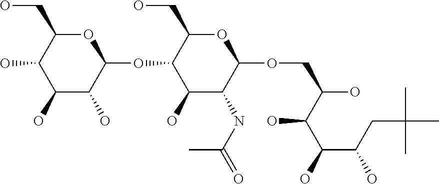 |
| 4-O-(6-O-[Aceta- mido-2-deoxy- beta-D-gluco- pyranosyl]-beta- D-galacto- pyranosyl)- | 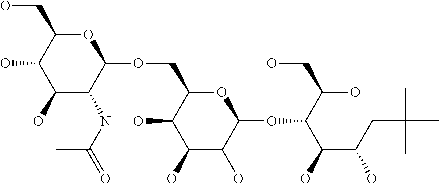 |
| D-Cellotetrao- | 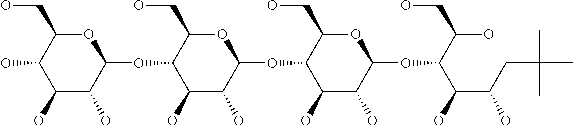 |
| Digalacturonic- | 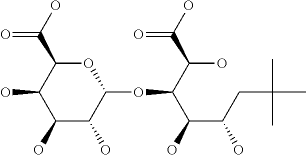 |
| 2′-Fucosyllacto- | 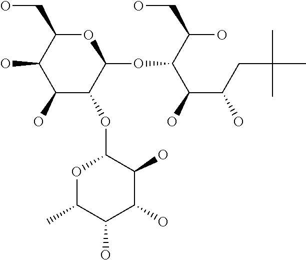 |
| 3-Fucosyllacto- | 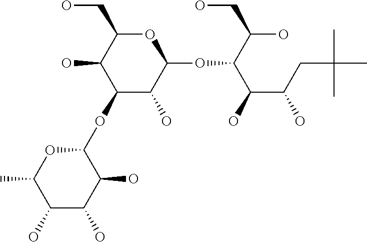 |
| Lacto-N-Tetrao- | 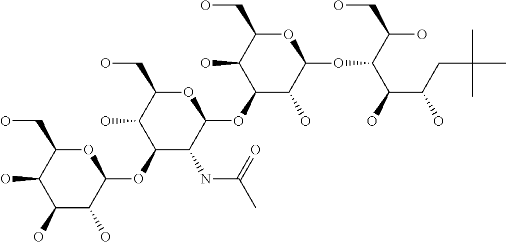 |
| 4-O-(2-O- Methyl-beta- D-galacto- pyrano- syl)-D-gluco- pyrano- | 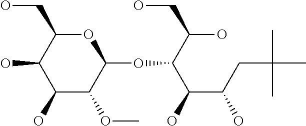 |
| A-Lactulo- | 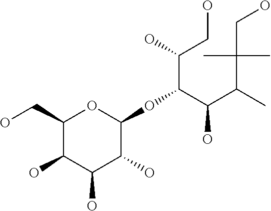 |
| Maltohexao- | 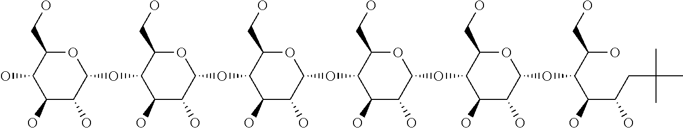 |
| L-Allo- | 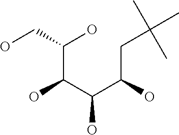 |
| 3-Deoxy- D-Gluco- | 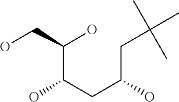 |
| Isomaltotetrao- | 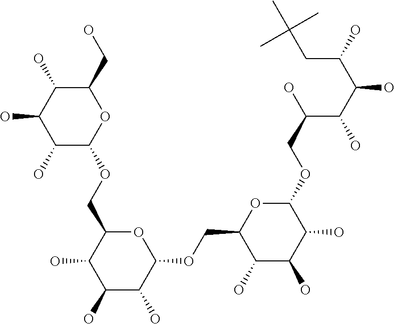 |
| Xylobio- | 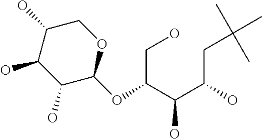 |
| Maltopentao- | 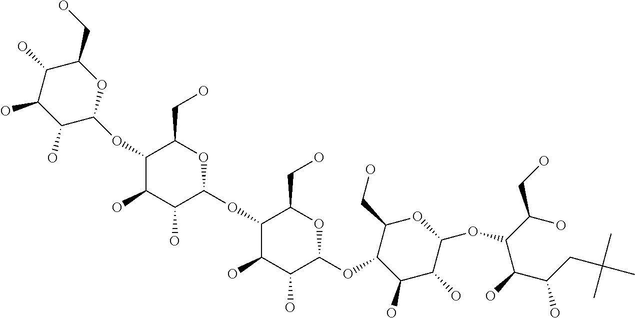 |
| Sophoro- | 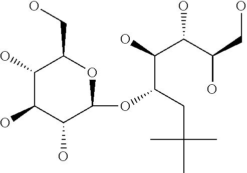 |
| D-Lacto- | 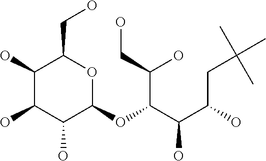 |
| 2-Acetamido-2- deoxy-3-O- (alpha-L-fuco- pyranosyl)-D- glucopyrano- | 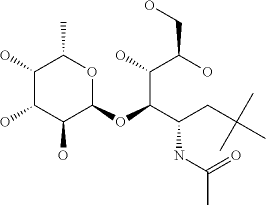 |
| 2-Acetamido-2- deoxy-4-O- (alpha-L-Fuco- pyranosyl)-D- glucopyrano- | 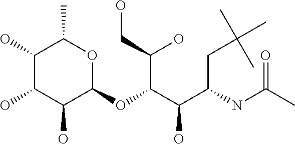 |
| D-Mannohepto- | 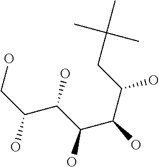 |
| Epilacto- | 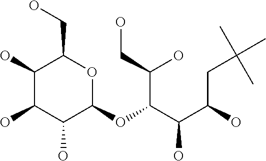 |
| Leucro- | 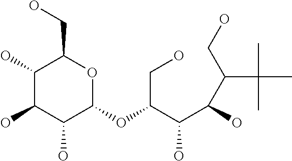 |
| A-Lactin- | 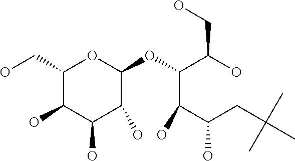 |
| Gantoobio- | 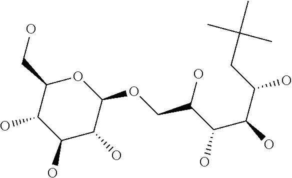 |
| D-Melibio- | 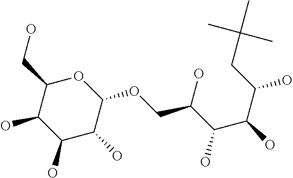 |
| Dimer-N-acetyl- galactosamin- | 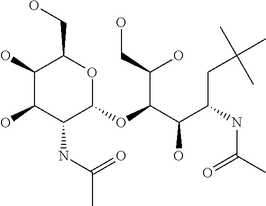 |
| 2-O-alpha-L- Fucosyl-D- galacto | 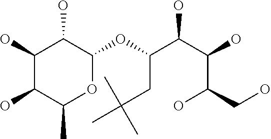 |
| Lactodifuco- tettrao- | 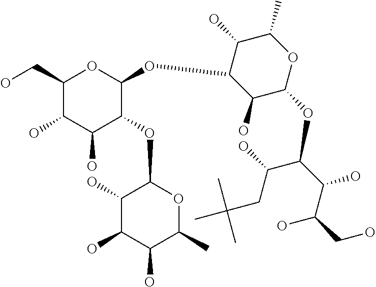 |
| 6-O-alpha-D- Mannopyranosyl- D-mannopyrano- | 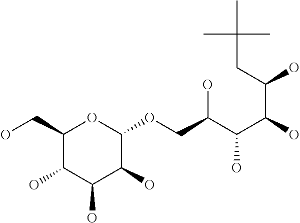 |
| 2-Acetamido-2- deoxy-6-O-(beta- D-galacto- pyranosyl)-D- galactopyrano- | 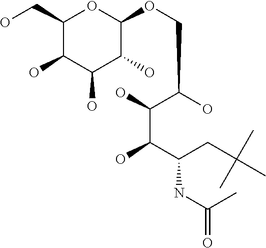 |
| D-Rhamno- | 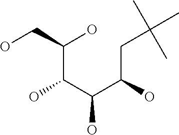 |
| D-Cellohexo- | 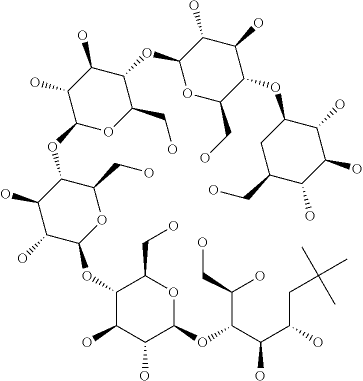 |
| L-Altro- | 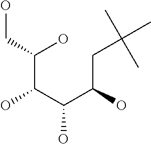 |
| 3-O-[2-Aceta- mido-2-deoxy- beta-D-gluco- pyranosyl]-D- mannopyrano- | 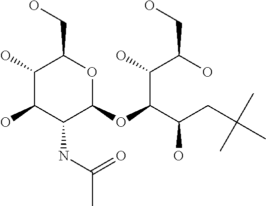 |
| 2-Deoxy-2- fluoro-D-manno- | 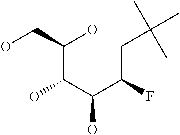 |
| 4-Deoxy-L-fuco- | 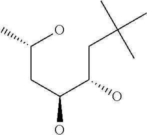 |
| 2-O-(alpha-D- galacto- pyranosyl)-D- galacto- | 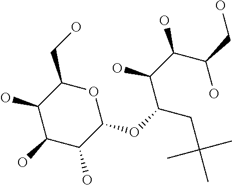 |
| 3-O-(alpha- D-Galacto- pyranosyl)-D- galacto- | 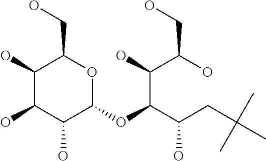 |
| D-Galacto- | 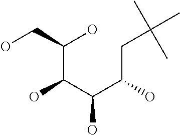 |
| Globotrio- | 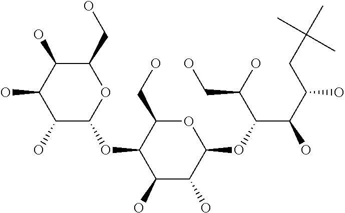 |
| 2-Acetamido-2- deoxy-4-O-beta- D-galacto- pyranosyl-D- mannopyrano- | 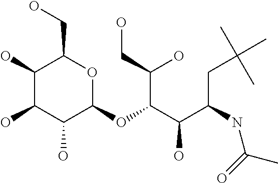 |
| 2-Acetamido-2- deoxy-4-O-(beta- D-manno- pyranosyl)-D- glucopyrano- | 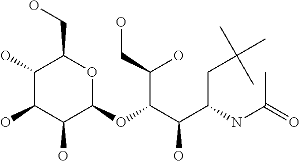 |
| 4-O-beta-D- galacto- pyranosyl-D- galactopyrano- | 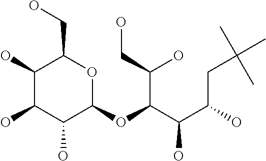 |
| 4-O-(3-O-alpha- D-Galacto- pyranosyl-beta- D-galacto- pyranosyl)-D- galactopyrano- | 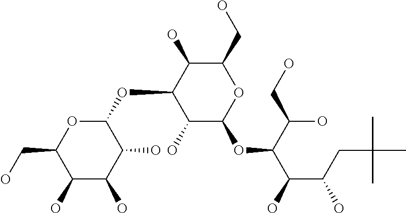 |
| A1-3, B1-4, A1-3 Galactotetrao- | 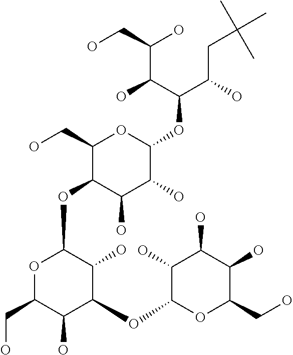 |
| 2-O-alpha-D- Mannopyranosyl- D-mannopyrano- | 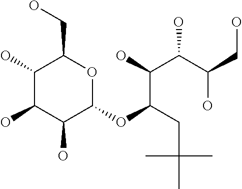 |
| 4-O-alpha-D- Mannopyranosyl- D-mannopyrano- | 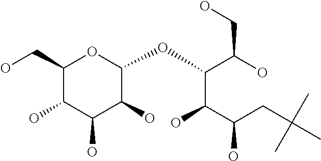 |
| 2-O-(2-Aceta- mido-2-deoxy- beta-D-gluco- pyranosyl)- D-manno- | 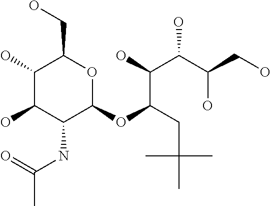 |
| 3-O-(alpha-L- Fucopyranosyl)- D-galacto- | 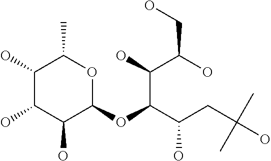 |
| 4-O-(alpha-L- Fucopyranosyl)- D-galacto- | 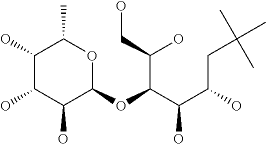 |
| 2′-Fucosyl-N- acetallactos-ami | 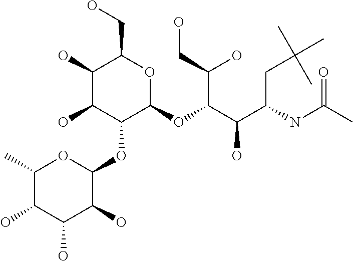 |
| Laminaritrio- | 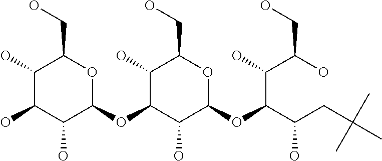 |
| Laminaritetrao- | 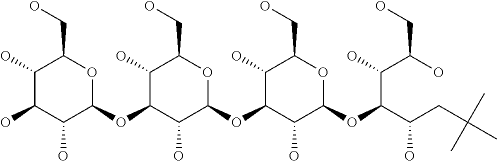 |
| Laminaripentao- | 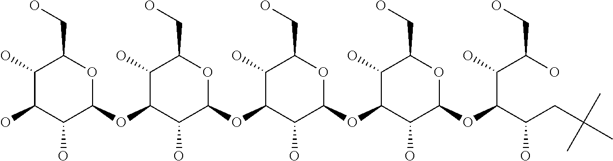 |
| Laminarihexao- | 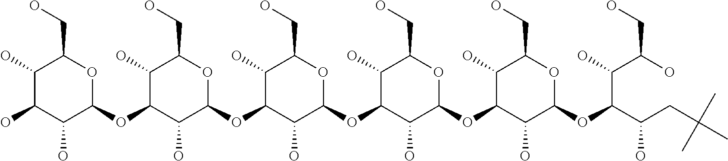 |
| Lacto-N-bio | 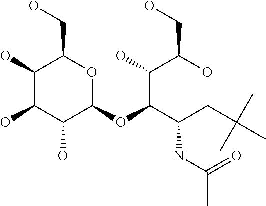 |
| A1-2-Mannobio- | 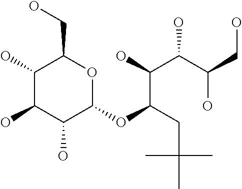 |
| A1-3, A1-6- Mannotrio- | 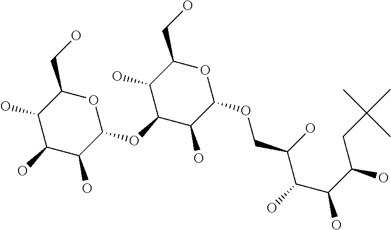 |
| A1-3, A1-6- Mannopentao- | 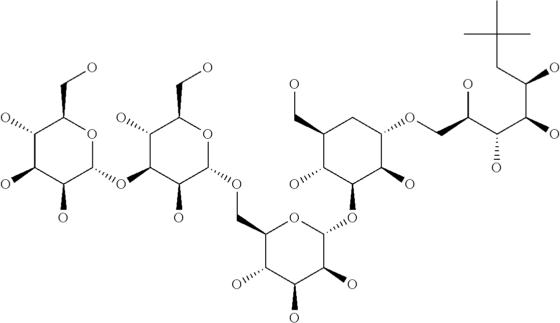 |
| 2-Acetamido-2- deoxy-3-O- methyl-D- glucopyranosi- | 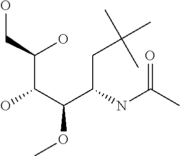 |
| Fucose alpha A1-2-galactose- beta A1,4-N- acetylglucosami- | 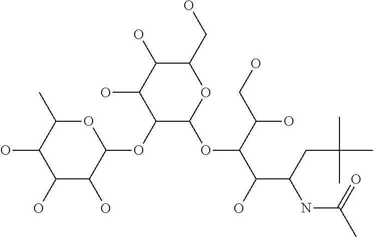 |
| Fucose alpha 1,6-N-acetylglu- cosami- | 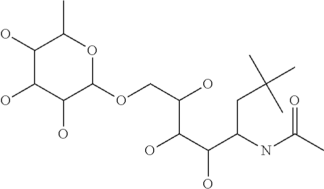 |
| Galactose beta 1,6-N-acetyl- glucosami- | 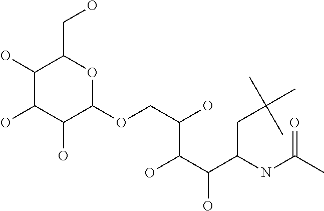 |
| D-Ribulo- | 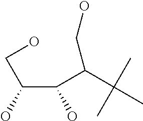 |
| D-Threo- | 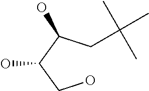 |
| Arabinic AC- | 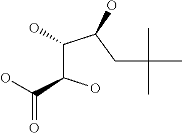 |
| Lactulo- | 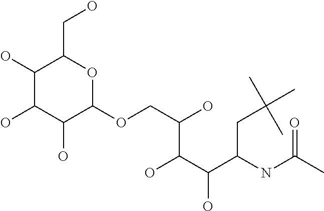 |
| L-Xylulo- | 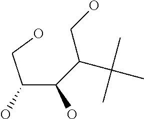 |
| D-Xylulo- | 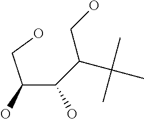 |
| D-Fructo- | 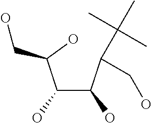 |
| L-Threo- | 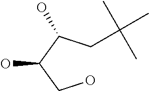 |
| 5-Deoxy-D-xylo- furano- | 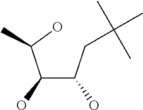 |
| 2-Fluoro-2- deoxy-D- arabino- | 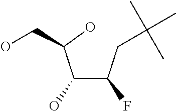 |
| Palatino- | 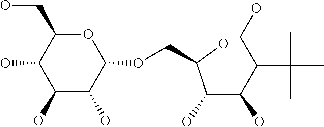 |
| 2-Deoxy-L-ribo- | 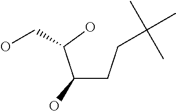 |
| | 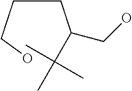 |
| Maltulo- | 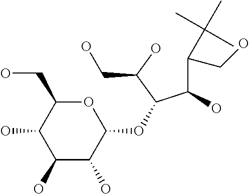 |
| Trehalulo- | 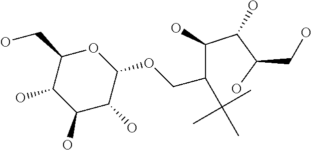 |
| D-Arabino- | 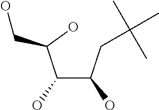 |
| L-Arabino- | 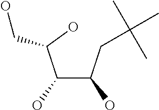 |
| D-Erythro- | 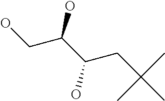 |
| L-Glycer- | 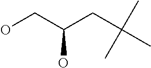 |
| L-Erythro- | 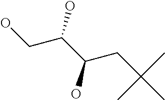 |
| D-Glycer- | 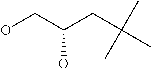 |
| L-Ribo- | 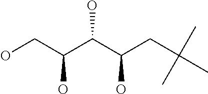 |
| D-Ribo- | 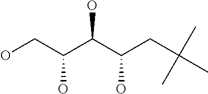 |
| D-Fuco- | 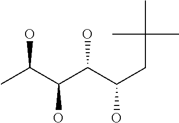 |
| D-Cellobio- | 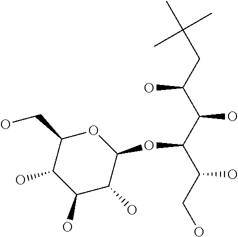 |
| 5-Deoxy-L- arabino- | 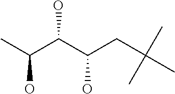 |
| D-Xylo- | 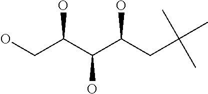 |
| L-Xylo- | 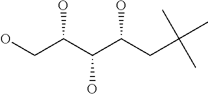 |
| Cellopentao- | 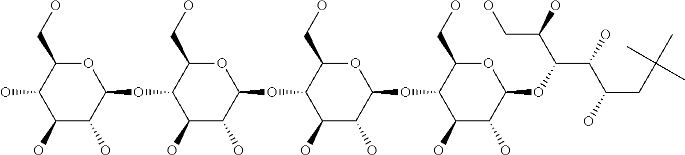 |
| Pano- | 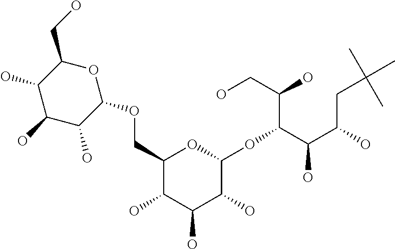 |
| Rutino- | 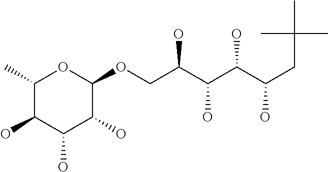 |
| Beta-Gentiobio- | 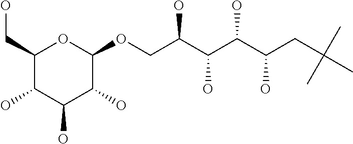 |
| 6-Deoxy-L-talo- | 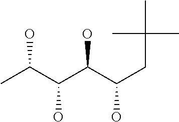 |
| L-Iduronic- | 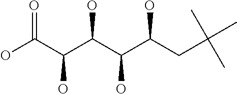 |
| L-Glycerol-L- galactohepto- | 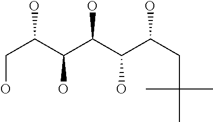 |
| L-Glycero-D- galactohepto- | 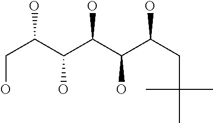 |
| D-Lacta- | 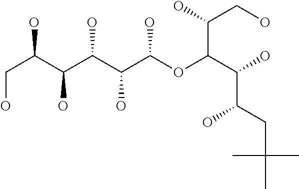 |
| Gluconic- | 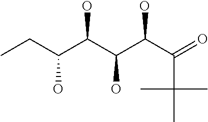 |
| 5-Ketogluconic- | 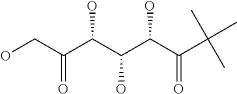 |
| Heptagluconic- | 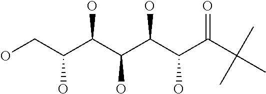 |
| Lactobionic- | 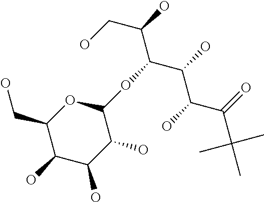 |
| D-Xylonic- | 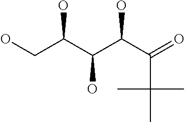 |
| Arabic- | 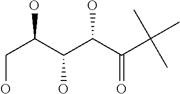 |

The term “C₁₋ₓ alkyl” denotes any linear or branched alkyl chain containing from 1 to x carbons.

The term “C₃₋₈ cycloalkyl” denotes carbocyclic saturated radicals containing from 3 to 8 carbons.

The term “aryl” stands for carbocyclic aromatics containing from 6 to 14 carbons, particularly phenyl, 1-naphthyl, and 2-naphthyl.

The term “heteroaryl” stands for five-ring and six-ring aromatics containing at least one hetero-atom N, O, or S, and particularly denotes pyridyl, thienyl, furyl, thiazolyl, and imidazolyl; two of the aromatic rings may be condensed, as in indole, N—(C₁₋₃ alkyl)indole, benzothiophene, benzothiazole, benzimidazole, quinoline, and isoquinoline.

The term “C_{x-y} alkylaryl” stands for carbocyclic aromatics that are linked to the skeleton through an alkyl group containing x, x+1 . . . y−1, or y carbons.

The compounds of formula I can exist as such or be in the form of their salts with physiologically acceptable acids. Examples of such acids are: hydrochloric acid, citric acid, tartaric acid, lactic acid, phosphoric acid, methanesulfonic acid, acetic acid, formic acid, maleic acid, fumaric acid, succinic acid, hydroxysuccinic acid, sulfuric acid, glutaric acid, aspartic acid, pyruvic acid, benzoic acid, glucuronic acid, oxalic acid, ascorbic acid, and acetylglycine.

The novel compounds of formula I are competitive inhibitors of thrombin or the complement system, especially C1s, and also C1r.

The compounds of the invention can be administered in conventional manner orally or parenterally (subcutaneously, intravenously, intramuscularly, intraperitoneally, or rectally). Administration can also be carried out with vapors or sprays applied to the postnasal space.

The dosage depends on the age, condition, and weight of the patient, and also on the method of administration used. Usually the daily dose of the active component per person is between approximately 10 and 2000 mg for oral administration and between approximately 1 and 200 mg for parenteral administration. These doses can take the form of from 2 to 4 single doses per day or be administered once a day as depot.

The compounds can be employed in commonly used galenic solid or liquid administration forms, eg, as tablets, film tablets, capsules, powders, granules, dragees, suppositories, solutions, ointments, creams, or sprays. These are produced in conventional manner. The active substances can be formulated with conventional galenic auxiliaries, such as tablet binders, fillers, preserving agents, tablet bursters, flow regulators, plasticizers, wetters, dispersing agents, emulsifiers, solvents, retarding agents, antioxidants, and/or fuel gases (cf H. Sucker et al.: Pharmazeutische Technologie, Thieme-Verlag, Stuttgart, 1978). The resulting administration forms normally contain the active substance in a concentration of from 0.1 to 99 wt %.

The term “prodrugs” refers to compounds which are converted to the pharmacologically active compounds of the general formula I in vivo (eg, first pass metabolisms).

Where, in the compounds of formula I, R^{L1} is not hydrogen, the respective substances are prodrugs from which the free amidine or guanidine compounds are formed under in vivo conditions. If ester functions are present in the compounds of formula I, these compounds can act, in vivo, as prodrugs, from which the corresponding carboxylic acids are formed.

Apart from the substances mentioned in the examples, the following compounds are very particularly preferred and can be produced according to said manufacturing instructions:

| | |
|---|---|
| 1. | L-Glycer-D-Cha-Pro-NH-4-amb |
| 2. | D-Glycer-D-Cha-Pro-NH-4-amb |
| 3. | L-Erythro-D-Cha-Pro-NH-4-amb |
| 4. | D-Erythro-D-Cha-Pro-NH-4-amb |
| 5. | L-Threo-D-Cha-Pro-NH-4-amb |
| 6. | D-Threo-D-Cha-Pro-NH-4-amb |
| 7. | L-Arabino-D-Cha-Pro-NH-4-amb |
| 8. | D-Arabino-D-Cha-Pro-NH-4-amb |
| 9. | L-Ribo-D-Cha-Pro-NH-4-amb |
| 10. | D-Ribo-D-Cha-Pro-NH-4-amb |
| 11. | 2-Deoxy-L-Ribo-D-Cha-Pro-NH-4-amb |
| 12. | D-Fuco-D-Cha-Pro-NH-4-amb |
| 13. | D-Cellobio-D-Cha-Pro-NH-4-amb |
| 14. | D-Xylo-D-Cha-Pro-NH-4-amb |
| 15. | L-Xylo-D-Cha-Pro-NH-4-amb |
| 16. | Cellopentao-D-Cha-Pro-NH-4-amb |
| 17. | D-Fructo-D-Cha-Pro-NH-4-amb |
| 18. | Maltotrio-D-Cha-Pro-NH-4-amb |
| 19. | Maltotetrao-D-Cha-Pro-NH-4-amb |
| 20. | Glucohepto-D-Cha-Pro-NH-4-amb |
| 21. | L-Allo-D-Cha-Pro-NH-4-amb |
| 22. | D-Allio-D-Cha-Pro-NH-4-amb |
| 23. | D-Gluco-D-Cha-Pro-NH-4-amb |
| 24. | L-Gluco-D-Cha-Pro-NH-4-amb |
| 25. | D-Manno-D-Cha-Pro-NH-4-amb |
| 26. | L-Manno-D-Cha-Pro-NH-4-amb |
| 27. | L-Galacto-D-Cha-Pro-NH-4-amb |
| 28. | Dextro-D-Cha-Pro-NH-4-amb |
| 29. | L-Lyxo-D-Cha-Pro-NH-4-amb |
| 30. | D-Lyxo-D-Cha-Pro-NH-4-amb |
| 31. | D-Lacto-D-Cha-Pro-NH-4-amb |
| 32. | D-Talo-D-Cha-Pro-NH-4-amb |
| 33. | L-Talo-D-Cha-Pro-NH-4-amb |
| 34. | beta-Malto-D-Cha-Pro-NH-4-amb |
| 35. | L-Fuco-D-Cha-Pro-NH-4-amb |
| 36. | L-Gulo-D-Cha-Pro-NH-4-amb |
| 37. | D-Gulo-D-Cha-Pro-NH-4-amb |
| 38. | L-ldo-D-Cha-Pro-NH-4-amb |
| 39. | D-ldo-D-Cha-Pro-NH-4-amb |
| 40. | D-Cellotrio-D-Cha-Pro-NH-4-amb |
| 41. | D-Galacturonic-D-Cha-Pro-NH-4-amb |
| 42. | D-Glucuronic-D-Cha-Pro-NH-4-amb |
| 43. | L-Rhamno-D-Cha-Pro-NH-4-amb |
| 44. | D-Cellotetrao-D-Cha-Pro-NH-4-amb |
| 45. | Maltohexao-D-Cha-Pro-NH-4-amb |
| 46. | Maltopentao-D-Cha-Pro-NH-4-amb |
| 47. | Xylobio-D-Cha-Pro-NH-4-amb |
| 48. | D-Lacto-D-Cha-Pro-NH-4-amb |
| 49. | D-Melibio-D-Cha-Pro-NH-4-amb |
| 50. | Gentobio-D-Cha-Pro-NH-4-amb |
| 51. | D-Rhamno-D-Cha-Pro-NH-4-amb |
| 52. | L-Altro-D-Cha-Pro-NH-4-amb |
| 53. | D-Galacto-D-Cha-Pro-NH-4-amb |
| 54. | L-Glycer-D-Chg-Ace-NH-4-amb |
| 55. | D-Glycer-D-Chg-Ace-NH-4-amb |
| 56. | L-Erythro-D-Chg-Ace-NH-4-amb |
| 57. | D-Erythro-D-Chg-Ace-NH-4-amb |
| 58. | L-Threo-D-Chg-Ace-NH-4-amb |
| 59. | D-Threo-D-Chg-Ace-NH-4-amb |
| 60. | L-Arabino-D-Chg-Ace-NH-4-amb |
| 61. | D-Arabino-D-Chg-Ace-NH-4-amb |
| 62. | L-Ribo-D-Chg-Ace-NH-4-amb |
| 63. | D-Ribo-D-Chg-Ace-NH-4-amb |
| 64. | 2-Deoxy-L-Ribo-D-Chg-Ace-NH-4-amb |
| 65. | D-Fuco-D-Chg-Ace-NH-4-amb |
| 66. | D-Cellobio-D-Chg-Ace-NH-4-amb |
| 67. | D-Xylo-D-Chg-Ace-NH-4-amb |
| 68. | L-Xylo-D-Chg-Ace-NH-4-amb |
| 69. | Cellopentao-D-Chg-Ace-NH-4-amb |
| 70. | D-Fructo-D-Chg-Ace-NH-4-amb |
| 71. | Maltotrio-D-Chg-Ace-NH-4-amb |
| 72. | Maltotetrao-D-Chg-Ace-NH-4-amb |
| 73. | Glucohepto-D-Chg-Ace-NH-4-amb |
| 74. | L-Allo-D-Chg-Ace-NH-4-amb |
| 75. | D-Allo-D-Chg-Ace-NH-4-amb |
| 76. | L-Gluco-D-Chg-Ace-NH-4-amb |
| 77. | D-Manno-D-Chg-Ace-NH-4-amb |
| 78. | L-Manno-D-Chg-Ace-NH-4-amb |
| 79. | L-Galacto-D-Chg-Ace-NH-4-amb |
| 80. | Dextro-D-Chg-Ace-NH-4-amb |
| 81. | L-Lyxo-D-Chg-Ace-NH-4-amb |
| 82. | D-Lyxo-D-Chg-Ace-NH-4-amb |
| 83. | D-Lacto-D-Chg-Ace-NH-4-amb |
| 84. | D-Talo-D-Chg-Ace-NH-4-amb |
| 85. | L-Talo-D-Chg-Ace-NH-4-amb |
| 86. | L-Fuco-D-Chg-Ace-NH-4-amb |
| 87. | L-Gulo-D-Chg-Ace-NH-4-amb |
| 88. | D-Gulo-D-Chg-Ace-NH-4-amb |
| 89. | L-Ido-D-Chg-Ace-NH-4-amb |
| 90. | D-Ido-D-Chg-Ace-NH-4-amb |
| 91. | D-Cellotrio-D-Chg-Ace-NH-4-amb |
| 92. | D-Galacturonic-D-Chg-Ace-NH-4-amb |
| 93. | D-Glucuronic-D-Chg-Ace-NH-4-amb |
| 94. | L-Rhamno-D-Chg-Ace-NH-4-amb |
| 95. | D-Cellotetrao-D-Chg-Ace-NH-4-amb |
| 96. | Maltohexao-D-Chg-Ace-NH-4-amb |
| 97. | Maltopentao-D-Chg-Ace-NH-4-amb |
| 98. | Xylobio-D-Chg-Ace-NH-4-amb |
| 99. | D-Lacto-D-Chg-Ace-NH-4-amb |
| 100. | D-Melibio-D-Chg-Ace-NH-4-amb |
| 101. | Gentobio-D-Chg-Ace-NH-4-amb |
| 102. | D-Rhamno-D-Chg-Ace-NH-4-amb |
| 103. | L-Altro-D-Chg-Ace-NH-4-amb |
| 104. | D-Galacto-D-Chg-Ace-NH-4-amb |
| 105. | L-Glycer-D-Cha-Pyr-NH-3-(6-am)-pico |
| 106. | D-Glycer-D-Cha-Pyr-NH-3-(6-am)-pico |
| 107. | L-Erythro-D-Cha-Pyr-NH-3-(6-am)-pico |
| 108. | D-Erythro-D-Cha-Pyr-NH-3-(6-am)-pico |
| 109. | L-Threo-D-Cha-Pyr-NH-3-(6-am)-pico |
| 110. | D-Threo-D-Cha-Pyr-NH-3-(6-am)-pico |
| 111. | L-Arabino-D-Cha-Pyr-NH-3-(6-am)-pico |
| 112. | D-Arabino-D-Cha-Pyr-NH-3-(6-am)-pico |
| 113. | L-Ribo-D-Cha-Pyr-NH-3-(6-am)-pico |
| 114. | D-Ribo-D-Cha-Pyr-NH-3-(6-am)-pico |
| 115. | 2-Deoxy-L-Ribo-D-Cha-Pyr-NH-3-(6-am)-pico |
| 116. | D-Fuco-D-Cha-Pyr-NH-3-(6-am)-pico |
| 117. | D-Cellobio-D-Cha-Pyr-NH-3-(6-am)-pico |
| 118. | D-Xylo-D-Cha-Pyr-NH-3-(6-am)-pico |
| 119. | L-Xylo-D-Cha-Pyr-NH-3-(6-am)-pico |
| 120. | Cellopentao-D-Cha-Pyr-NH-3-(6-am)-pico |
| 121. | D-Fructo-D-Cha-Pyr-NH-3-(6-am)-pico |
| 122. | Maltotrio-D-Cha-Pyr-NH-3-(6-am)-pico |
| 123. | Maltotetrao-D-Cha-Pyr-NH-3-(6-am)-pico |
| 124. | Glucohepto-D-Cha-Pyr-NH-3-(6-am)-pico |
| 125. | L-Allo-D-Cha-Pyr-NH-3-(6-am)-pico |
| 126. | D-Allo-D-Cha-Pyr-NH-3-(6-am)-pico |
| 127. | D-Gluco-D-Cha-Pyr-NH-3-(6-am)-pico |
| 128. | L-Gluco-D-Cha-Pyr-NH-3-(6-am)-pico |
| 129. | D-Manno-D-Cha-Pyr-NH-3-(6-am)-pico |
| 130. | L-Manno-D-Cha-Pyr-NH-3-(6-am)-pico |
| 131. | L-Galacto-D-Cha-Pyr-NH-3-(6-am)-pico |
| 132. | Dextro-D-Cha-Pyr-NH-3-(6-am)-pico |
| 133. | L-Lyxo-D-Cha-Pyr-NH-3-(6-am)-pico |
| 134. | D-Lyxo-D-Cha-Pyr-NH-3-(6-am)-pico |
| 135. | D-Lacto-D-Cha-Pyr-NH-3-(6-am)-pico |
| 136. | D-Talo-D-Cha-Pyr-NH-3-(6-am)-pico |
| 137. | L-Talo-D-Cha-Pyr-NH-3-(6-am)-pico |
| 138. | beta-Malto-D-Cha-Pyr-NH-3-(6-am)-pico |
| 139. | L-Fuco-D-Cha-Pyr-NH-3-(6-am)-pico |
| 140. | L-Gulo-D-Cha-Pyr-NH-3-(6-am)-pico |
| 141. | D-Gulo-D-Cha-Pyr-NH-3-(6-am)-pico |
| 142. | L-ldo-D-Cha-Pyr-NH-3-(6-am)-pico |
| 143. | D-Ido-D-Cha-Pyr-NH-3-(6-am)-pico |
| 144. | D-Cellotrio-D-Cha-Pyr-NH-3-(6-am)-pico |
| 145. | D-Galacturonic-D-Cha-Pyr-NH-3-(6-am)-pico |
| 146. | D-Glucuronic-D-Cha-Pyr-NH-3-(6-am)-pico |
| 147. | L-Rhamno-D-Cha-Pyr-NH-3-(6-am)-pico |
| 148. | D-Cellotetrao-D-Cha-Pyr-NH-3-(6-am)-pico |
| 149. | Maltohexao-D-Cha-Pyr-NH-3-(6-am)-pico |
| 150. | Maltopentao-D-Cha-Pyr-NH-3-(6-am)-pico |
| 151. | Xylobio-D-Cha-Pyr-NH-3-(6-am)-pico |
| 152. | D-Lacto-D-Cha-Pyr-NH-3-(6-am)-pico |
| 153. | D-Melibio-D-Cha-Pyr-NH-3-(6-am)-pico |
| 154. | Gentobio-D-Cha-Pyr-NH-3-(6-am)-pico |
| 155. | D-Rhamno-D-Cha-Pyr-NH-3-(6-am)-pico |
| 156. | L-Altro-D-Cha-Pyr-NH-3-(6-am)-pico |
| 157. | D-Galacto-D-Cha-Pyr-NH-3-(6-am)-pico |
| 158. | L-Erythro-D-Cha-Pyr-NH—CH₂-2-(4-am)-thiaz |
| 159. | D-Threo-D-Cha-Pyr-NH—CH₂-2-(4-am)-thiaz |
| 160. | L-Ribo-D-Cha-Pyr-NH—CH₂-2-(4-am)-thiaz |
| 161. | D-Ribo-D-Cha-Pyr-NH—CH₂-2-(4-am)-thiaz |
| 162. | 2-Deoxy-L-Ribo-D-Cha-Pyr-NH—CH₂-2-(4-am)-thiaz |
| 163. | D-Fuco-D-Cha-Pyr-NH—CH₂-2-(4-am)-thiaz |
| 164. | D-Cellobio-D-Cha-Pyr-NH—CH₂-2-(4-am)-thiaz |
| 165. | D-Xylo-D-Cha-Pyr-NH—CH₂-2-(4-am)-thiaz |
| 166. | L-Xylo-D-Cha-Pyr-NH—CH₂-2-(4-am)-thiaz |
| 167. | Cellopentao-D-Cha-Pyr-NH—CH₂-2-(4-am)-thiaz |
| 168. | D-Fructo-D-Cha-Pyr-NH—CH₂-2-(4-am)-thiaz |
| 169. | Maltotrio-D-Cha-Pyr-NH—CH₂-2-(4-am)-thiaz |
| 170. | Maltotetrao-D-Cha-Pyr-NH—CH₂-2-(4-am)-thiaz |
| 171. | Glucohepto-D-Cha-Pyr-NH—CH₂-2-(4-am)-thiaz |
| 172. | L-Allo-D-Cha-Pyr-NH—CH₂-2-(4-am)-thiaz |
| 173. | D-Allo-D-Cha-Pyr-NH—CH₂-2-(4-am)-thiaz |
| 174. | D-Gluco-D-Cha-Pyr-NH—CH₂-2-(4-am)-thiaz |
| 175. | L-Gluco-D-Cha-Pyr-NH—CH₂-2-(4-am)-thiaz |
| 176. | D-Manno-D-Cha-Pyr-NH—CH₂-2-(4-am)-thiaz |
| 177. | L-Manno-D-Cha-Pyr-NH—CH₂-2-(4-am)-thiaz |
| 178. | L-Galacto-D-Cha-Pyr-NH—CH₂-2-(4-am)-thiaz |
| 179. | Dextro-D-Cha-Pyr-NH—CH₂-2-(4-am)-thiaz |
| 180. | L-Lyxo-D-Cha-Pyr-NH—CH₂-2-(4-am)-thiaz |
| 181. | D-Lyxo-D-Cha-Pyr-NH—CH₂-2-(4-am)-thiaz |
| 182. | D-Lacto-D-Cha-Pyr-NH—CH₂-2-(4-am)-thiaz |
| 183. | D-Talo-D-Cha-Pyr-NH—CH₂-2-(4-am)-thiaz |
| 184. | L-Talo-D-Cha-Pyr-NH—CH₂-2-(4-am)-thiaz |
| 185. | beta-Maltro-D-Cha-Pyr-NH—CH₂-2-(4-am)-thiaz |
| 186. | L-Fuco-D-Cha-Pyr-NH—CH₂-2-(4-am)-thiaz |
| 187. | L-Gulo-D-Cha-Pyr-NH—CH₂-2-(4-am)-thiaz |
| 188. | D-Gulo-D-Cha-Pyr-NH—CH₂-2-(4-am)-thiaz |
| 189. | L-Ido-D-Cha-Pyr-NH—CH₂-2-(4-am)-thiaz |
| 190. | D-ldo-D-Cha-Pyr-NH—CH₂-2-(4-am)-thiaz |
| 191. | D-Cellotrio-D-Cha-Pyr-NH—CH₂-2-(4-am)-thiaz |
| 192. | D-Galacturonic-D-Cha-Pyr-NH—CH₂-2-(4-am)-thiaz |
| 193. | D-Glucuronic-D-Cha-Pyr-NH—CH₂-2-(4-am)-thiaz |
| 194. | D-Cellotetrao-D-Cha-Pyr-NH—CH₂-2-(4-am)-thiaz |
| 195. | Maltohexao-D-Cha-Pyr-NH—CH₂-2-(4-am)-thiaz |
| 196. | Maltopentao-D-Cha-Pyr-NH—CH₂-2-(4-am)-thiaz |
| 197. | Xylobio-D-Cha-Pyr-NH—CH₂-2-(4-am)-thiaz |
| 198. | D-Lacto-D-Cha-Pyr-NH—CH₂-2-(4-am)-thiaz |
| 199. | Gentobio-D-Cha-Pyr-NH—CH₂-2-(4-am)-thiaz |
| 200. | D-Rhamno-D-Cha-Pyr-NH—CH₂-2-(4-am)-thiaz |
| 201. | L-Altro-D-Cha-Pyr-NH—CH₂-2-(4-am)-thiaz |
| 202. | D-Galacto-D-Cha-Pyr-NH—CH₂-2-(4-am)-thiaz |
| 203. | D-Galacturo-D-Cha-Pyr-NH—CH₂-2-(4-am)-thiaz |
| 205. | D-Glucohepto-D-Cha-Pyr-NH—CH₂-2-(4-am)-thiaz |
| 206. | L-Allo-D-Cha-Pyr-NH—CH₂-2-(4-am)-thiaz |
| 207. | D-Allo-D-Cha-Pyr-NH—CH₂-2-(4-am)-thiaz |
| 208. | D-Gluco-D-Cha-Pyr-NH—CH₂-2-(4-am)-thiaz |
| 209. | D-Galacto-D-Cha-Pyr-NH—CH₂-2-(4-am)-thiaz |
| 210. | L-Gluco-D-Cha-Pyr-NH—CH₂-2-(4-am)-thiaz |
| 211. | L-Manno-D-Cha-Pyr-NH—CH₂-2-(4-am)-thiaz |
| 212. | D-Manno-D-Cha-Pyr-NH—CH₂-2-(4-am)-thiaz |
| 213. | D-Cellotrio-D-Cha-Pyr-NH—CH₂-2-(4-am)-thiaz |
| 214. | D-Cellobio-D-Cha-Pyr-NH—CH₂-2-(4-am)-thiaz |
| 215. | D-Glucuronic-D-Cha-Pyr-NH—CH₂-2-(4-am)-thiaz |
| 216. | Arabinic AC-D-Cha-Pyr-NH—CH₂-2-(4-am)-thiaz |
| 217. | L-lduronic-D-Cha-Pyr-NH—CH₂-2-(4-am)-thiaz |
| 218. | Gluconlc-D-Cha-Pyr-NH—CH₂-2-(4-am)-thiaz |
| 219. | Heptagluconic-D-Cha-Pyr-NH—CH₂-2-(4-am)-thiaz |
| 220. | Lactobionic-D-Cha-Pyr-NH—CH₂-2-(4-am)-thiaz |
| 221. | D-Xylonic-D-Cha-Pyr-NH—CH₂-2-(4-am)-thiaz |
| 222. | Arabic-D-Cha-Pyr-NH—CH₂-2-(4-am)-thiaz |
| 223. | Phenyl-beta-D-Glucuronic-D-Cha-Pyr-NH—CH₂-2-(4-am)-thiaz |
| 224. | Methyl-beta-D-Glucuronic-D-Cha-Pyr-NH—CH₂-2-(4-am)-thiaz |
| 225. | D-quinic-D-Cha-Pyr-NH—CH₂-2-(4-am)-thiaz |
| 226. | Phenyl-alpha-iduronic-D-Cha-Pyr-NH—CH₂-2-(4-am)-thiaz |
| 227. | Digalacturonlc-D-Cha-Pyr-NH—CH₂-2-(4-am)-thiaz |
| 228. | Trigalacturonic-D-Cha-Pyr-NH—CH₂-2-(4-am)-thiaz |
| 229. | 3,4,5-Trihydroxy-6-hydroxymethy-tetrahydropyranyl(2)-CO-D-Cha-Pyr-NH—CH₂-2-(4-am)-thiaz |
| 230. | 3-Acetamido-4,5-dihydroxy-6-hydroxymethyl-tetrahydropyanyl(2)-CO-D-Cha-Pyr-NH—CH₂-2-(4-am)-thiaz |
| 231. | D-Galacturo-NH-cyclohexyl-CO-D-Cha-Pyr-NH—CH₂-2-(4-am)-thiaz |
| 232. | D-Glucohepto-NH-cyclohexyl-CO-D-Cha-Pyr-NH—CH₂-2-(4-am)-thiaz |
| 233. | L-Allo-NH-cyclohexyl-CO-D-Cha-Pyr-NH—CH₂-2-(4-am)-thiaz |
| 234. | D-Allo-NH-cyclohexyl-CO-D-Cha-Pyr-NH—CH₂-2-(4-am)-thiaz |
| 235. | D-Gluco-NH-cyclohexyl-CO-D-Cha-Pyr-NH—CH₂-2-(4-am)-thiaz |
| 236. | D-Galacto-NH-cyclohexyl-CO-D-Cha-Pyr-NH—CH₂-2-(4-am)-thiaz |
| 237. | L-Gluco-NH-cyclohexyl-CO-D-Cha-Pyr-NH—CH₂-2-(4-am)-thiaz |
| 238. | L-Manna-NH-cyclohexyl-CO-D-Cha-Pyr-NH—CH₂-2-(4-am)-thiaz |
| 239. | D-Manno-NH-cyclohexyl-O-D-Cha-Pyr-NH—CH₂-2-(4-am)-thiaz |
| 240. | D-Cellotrio-NH-cyclohexyl-CO-D-Cha-Pyr-NH—CH₂-2-(4-am)-thiaz |
| 241. | D-Cellobio-NH-cyclohexyl-CO-D-Cha-Pyr-NH—CH₂-2-(4-am)-thiaz |
| 242. | D-Glucuronic-NH-cyclohexyl-CO-D-Cha-Pyr-NH—CH₂-2-(4-am)-thiaz |
| 243. | Arabinic AC-NH-cyclohexyl-CO-D-Cha-Pyr-NH—CH₂-2-(4-am)-thiaz |
| 244. | L-Iduronic-NH-cyclohexyl-CO-D-Cha-Pyr-NH—CH₂-2-(4-am)-thiaz |
| 245. | Gluconic-NH-cyclohexyl-CO-D-Cha-Pyr-NH—CH₂-2-(4-am)-thiaz |
| 246. | Heptagluconic-NH-cyclohexyl-CO-D-Cha-Pyr-NH—CH₂-2-(4-am)-thiaz |
| 247. | Lactoblonlc-NH-cyclohexyl-CO-D-Cha-Pyr-NH—CH₂-2-(4-am)-thiaz |
| 248. | D-Xylonic-NH-cyclohexyl-CO-D-Cha-Pyr-NH—CH₂-2-(4-am)-thiaz |
| 249. | Arabic-NH-cyclohexyl-CO-D-Cha-Pyr-NH—CH₂-2-(4-am)-thiaz |
| 250. | Pheny-beta-D-Glucuronic-NH-cyclohexyl-CO-D-Cha-Pyr-NH—CH₂-2-(4-am)-thiaz |
| 251. | Methyl-beta-D-Glucuronic-NH-cyclohexyl-CO-D-Cha-Pyr-NH—CH₂-2-(4-am)-thiaz |
| 252. | D-quinic-NH-cyclohexyl-CO-D-Cha-Pyr-NH—CH₂-2-(4-am)-thiaz |
| 253. | Phenyl-alpha-iduronic-NH-cyclohexyl-CO-D-Cha-Pyr-NH—CH₂-2-(4-am)-thiaz |
| 254. | Digalacturonic-NH-cyclohexyl-CO-D-Cha-Pyr-NH—CH₂-2-(4-am)-thiaz |
| 255. | Trigalacturonic-NH-cyclohexyl-CO-D-Cha-Pyr-NH—CH₂-2-(4-am)-thiaz |
| 256. | 3,4,5-trihydroxy-6-hydroxymethyl-tetrahydropyranyl(2)-CO—NH-cyclohexyl-CO-D-Cha-Pyr- |
| | NH—CH₂-2-(4-am)-thiaz |
| 257. | 3-acetamido-4,5-dihydroxy-6-hydroxymethyl-tetrahydropyranyl(2)-CO—NH-cyclohexyl-CO-D- |
| | Cha-Pyr-NH—CH₂-2-(4-am)-thiaz |
| 258. | D-Galacturo-NH—CH₂-p-phenyl-CO-D-Cha-Pyr-NH—CH₂-2-(4-am)-thiaz |
| 259. | D-Glucohepto-NH—CH₂-p-phenyl-CO-D-Cha-Pyr-NH—CH₂-2-(4-am)-thiaz |
| 260. | L-Allo-NH—CH₂-p-phenyl-CO-D-Cha-Pyr-NH—CH₂-2-(4-am)-thiaz |
| 261. | D-Allo-NH—CH₂-p-phenyl-CO-D-Cha-Pyr-NH—CH₂-2-(4-am)-thiaz |
| 262. | D-Gluco-NH—CH₂-p-phenyl-CO-D-Cha-Pyr-NH—CH₂-2-(4-am)-thiaz |
| 263. | D-Galacto-NH—CH₂-p-phenyl-CO-D-Cha-Pyr-NH—CH₂-2-(4-am)-thiaz |
| 264. | L-Gluco-NH—CH₂-p-phenyl-CO-D-Cha-Pyr-NH—CH₂-2-(4-am)-thiaz |
| 265. | L-Manno-NH—CH₂-p-phenyl-CO-D-Cha-Pyr-NH—CH₂-2-(4-am)-thiaz |
| 266. | D-Manno-NH—CH₂-p-phenyl-CO-D-Cha-Pyr-NH—CH₂-2-(4-am)-thiaz |
| 267. | D-Cellotrio-NH—CH₂-p-phenyl-CO-D-Cha-Pyr-NH—CH₂-2-(4-am)-thiaz |
| 268. | D-Cellobio-NH—CH₂-p-phenyl-CO-D-Cha-Pyr-NH—CH₂-2-(4-am)-thiaz |
| 269. | D-Glucuronic-NH—CH₂-p-phenyl-CO-D-Cha-Pyr-NH—CH₂-2-(4-am)-thiaz |
| 270. | Arabinic AC-NH—CH₂-p-phenyl-CO-D-Cha-Pyr-NH—CH₂-2-(4-am)-thiaz |
| 271. | L-lduronic-NH—CH₂-p-phenyl-CO-D-Cha-Pyr-NH—CH₂-2-(4-am)-thiaz |
| 272. | Gluconic-NH—CH₂-p-phenyl-CO-D-Cha-Pyr-NH—CH₂-2-(4-am)-thiaz |
| 273. | Heptagluconic-NH—CH₂-p-phenyl-CO-D-Cha-Pyr-NH—CH₂-2-(4-am)-thiaz |
| 274. | Lactobionic-NH—CH₂-p-phenyl-CO-D-Cha-Pyr-NH—CH₂-2-(4-am)-thiaz |
| 275. | D-Xylonic-NH—CH₂-p-phenyl-CO-D-Cha-Pyr-NH—CH₂-2-(4-am)-thiaz |
| 276. | Arabic-NH—CH₂-p-phenyl-CO-D-Cha-Pyr-NH—CH₂-2-(4-am)-thiaz |
| 277. | Phenyl-beta-D-Glucuronic-NH—CH₂-p-phenyl-CO-D-Cha-Pyr-NH—CH₂-2-(4-am)-thiaz |
| 278. | Methyl-beta-D-Glucuronic-NH—CH₂-p-phenyl-CO-D-Cha-Pyr-NH—CH₂-2-(4-am)-thiaz |
| 279. | D-quinic-NH—CH₂-p-phenyl-CO-D-Cha-Pyr-NH—CH₂-2-(4-am)-thiaz |
| 280. | Phenyl-alpha-iduronic-NH—CH₂-p-phenyl-CO-D-Cha-Pyr-NH—CH₂-2-(4-am)-thiaz |
| 281. | Digalacturonlc-NH—CH₂-p-phenyl-CO-D-Cha-Pyr-NH—CH₂-2-(4-am)-thiaz |
| 282. | Trigalacturonic-NH—CH₂-p-phenyl-CO-D-Cha-Pyr-NH—CH₂-2-(4-am)-thiaz |
| 283. | 3,4,5-Trihydroxy-6-hydroxymethyl-tetrahydropyranyl(2)-CONH—CH₂-p-phenyl-CO-D-Cha- |
| | Pyr-NH—CH₂-2-(4-am)-thiaz |
| 284. | 3-Acetamldo-4,5-dihydroxy-6-hydroxyinethyl-tetrahydropyranyl(2)-CONH—CH₂-p-phenyl-CO- |
| | D-Cha-Pyr-NH—CH₂-2-(4-am)-thiaz |
| 285. | D-Galacturo-NH—CH₂-p-phenyl-CH₂—CO-D-Cha-Pyr-NH—CH₂-2-(4-am)-thiaz |
| 286. | D-Glucohepto-NH—CH₂-p-phenyl-CH₂—CO-D-Cha-Pyr-NH—CH₂-2-(4-am)-thiaz |
| 287. | L-Allo-NH—CH₂-p-phenyl-CH₂—CO-D-Cha-Pyr-NH—CH₂-2-(4-am)-thiaz |
| 288. | D-Allo-NH—CH₂-p-phenyl-CH₂—CO-D-Cha-Pyr-NH—CH₂-2-(4-am)-thiaz |
| 289. | D-Gluco-NH—CH₂-p-phenyl-CH₂—CO-D-Cha-Pyr-NH—CH₂-2-(4-am)-thiaz |
| 290. | D-Galacto-NH—CH₂-p-phenyl-CH₂—CO-D-Cha-Pyr-NH—CH₂-2-(4-am)-thiaz |
| 291. | L-Gluco-NH—CH₂-p-phenyl-CH₂—CO-D-Cha-Pyr-NH—CH₂-2-(4-am)-thiaz |
| 292. | L-Manno-NH—CH₂-p-phenyl-CH₂-D-Cha-Pyr-NH—CH₂-2-(4-am)-thiaz |
| 293. | D-Manno-NH—CH₂-p-phenyl-CH₂—CO-D-Cha-Pyr-NH—CH₂-2-(4-am)-thiaz |
| 294. | D-Cellotrio-NH—CH₂-p-phenyl-CH₂—CO-D-Cha-Pyr-NH—CH₂-2-(4-am)-thiaz |
| 295. | D-Cellobio-NH—CH₂-p-phenyl-CH₂—CO-D-Cha-Pyr-NH—CH₂-2-(4-am)-thiaz |
| 296. | D-Glucuronic-NH—CH₂-p-phenyl-CH₂—CO-D-Cha-Pyr-NH—CH₂-2-(4-am)-thiaz |
| 297. | Arabinic AC-NH—CH₂-p-phenyl-CH₂—CO-D-Cha-Pyr-NH—CH₂-2-(4-am)-thiaz |
| 298. | L-lduronlc-NH—CH₂-p-phenyl-CH₂—CO-D-Cha-Pyr-NH—CH₂-2-(4-am)-thiaz |
| 299. | Gluconic-NH—CH₂-p-phenyl-CH₂—CO-D-Cha-Pyr-NH—CH₂-2-(4-am)-thiaz |
| 300. | Heptagluconic-NH—CH₂-p-phenyl-CH₂—CO-D-Cha-Pyr-NH—CH₂-2-(4-am)-thiaz |
| 301. | Lactobionic-NH—CH₂-p-phenyl-CH₂—CO-D-Cha-Pyr-NH—CH₂-2-(4-am)-thiaz |
| 302. | D-Xylonic-NH—CH₂-p-phenyl-CH₂—CO-D-Cha-Pyr-NH—CH₂-2-(4-am)-thiaz |
| 303. | Arabic-NH—CH₂-p-phenyl-CH₂—CO-D-Cha-Pyr-NH—CH₂-2-(4-am)-thiaz |
| 304. | Phenyl-beta-D-Glucuronic-NH—CH₂-p-phenyl-CH₂—CO-D-Cha-Pyr-NH—CH₂-2-(4-am)-thiaz |
| 305. | Methyl-beta-D-Glucuronic-NH—CH₂-p-phenyl-CH₂—CO-D-Cha-Pyr-NH—CH₂-2-(4-am)-thiaz |
| 306. | D-quinic-NH—CH₂-p-phenyl-CH₂—CO-D-Cha-Pyr-NH—CH₂-2-(4-am)-thiaz |
| 307. | Phenyl-alpha-Iduronic-NH—CH₂-p-phenyl-CH₂—CO-D-Cha-Pyr-NH—CH₂-2-(4-am)-thiaz |
| 308. | Digalacturonic-NH—CH₂-p-phenyl-CH₂—CO-D-Cha-Pyr-NH—CH₂-2-(4-am)-thiaz |
| 309. | Trigalacturonic-NH—CH₂-p-phenyl-CH₂—CO-D-Cha-Pyr-NH—CH₂-2-(4-am)-thiaz |
| 310. | 3,4,5-Trihydroxy-6-hydroxymethyl-tetrahydropyranyl(2)-CO—NH—CH₂-p-phenyl-CH₂—CO-D- |
| | Cha-Pyr-NH—CH₂-2-(4-am)-thiaz |
| 311. | 3-Acetamido-4,5-dihydroxy-6-hydroxymethyl-tetrahydropyranyl(2)-CO—NH—CH₂-p-phenyl- |
| | CH₂—CO-D-Cha-Pyr-NH—CH₂-2-(4-am)-thiaz |
| 312. | D-Galacturo-NH-p-pheny)-CH₂—CO-D-Cha-Pyr-NH—CH₂-2-(4-am)-thiaz |
| 313. | D-Glucohepto-NH-p-phenyl-CH₂—CO-D-Cha-Pyr-NH—CH₂-2-(4-am)-thiaz |
| 314. | L-Allo-NH-p-phenyl-CH₂—CO-D-Cha-Pyr-NH—CH₂-2-(4-am)-thiaz |
| 315. | D-Allo-NH-p-phenyl-CH₂—CO-D-Cha-Pyr-NH—CH₂-2-(4-am)-thiaz |
| 316. | D-Gluco-NH-p-phenyl-CH₂—CO-D-Cha-Pyr-NH—CH₂-2-(4-am)-thiaz |
| 317. | D-Galacto-NH-p-phenyl-CH₂—CO-D-Cha-Pyr-NH—CH₂-2-(4-am)-thiaz |
| 318. | L-Gluco-NH-p-phenyl-CH₂—CO-D-Cha-Pyr-NH—CH₂-2-(4-am)-thiaz |
| 319. | L-Manno-NH-p-phenyl-CH₂—CO-D-Cha-Pyr-NH—CH₂-2-(4-am)-thiaz |
| 320. | D-Manno-NH-p-phenyl-CH₂—CO-D-Cha-Pyr-NH—CH₂-2-(4-am)-thiaz |
| 321. | D-Cellotrio-NH-p-phenyl-CH₂—CO-D-Cha-Pyr-NH—CH₂-2-(4-am)-thiaz |
| 322. | D-Cellobio-NH-p-phenyl-CH₂—CO-D-Cha-Pyr-NH—CH₂-2-(4-am)-thiaz |
| 323. | D-Glucuronic-NH-p-phenyl-CH₂—CO-D-Cha-Pyr-NH—CH₂-2-(4-am)-thiaz |
| 324. | Arabinic AC-NH-p-phenyl-CH₂—CO-D-Cha-Pyr-NH—CH₂-2-(4-am)-thiaz |
| 325. | L-lduronic-NH-p-phenyl-CH₂—CO-D-Cha-Pyr-NH—CH₂-2-(4-am)-thiaz |
| 326. | Gluconic-NH-p-phenyl-CH₂—CO-D-Cha-Pyr-NH—CH₂-2-(4-am)-thiaz |
| 327. | Heptagluconic-NH-p-phenyl-CH₂—CO-D-Cha-Pyr-NH—CH₂-2-(4-am)-thiaz |
| 328. | Lactobionlc-NH-p-phenyl-CH₂—CO-D-Cha-Pyr-NH—CH₂-2-(4-am)-thiaz |
| 329. | D-Xylonic-NH-p-phenyl-CH₂—CO-D-Cha-Pyr-NH—CH₂-2-(4-am)-thiaz |
| 330. | Arabic-NH-p-phenyl-CH₂—CO-D-Cha-Pyr-NH—CH₂-2-(4-am)-thiaz |
| 331. | Phenyt-beta-D-Glucuronic-NH-p-phenyl-CH₂—CO-D-Cha-Pyr-NH—CH₂-2-(4-am)-thiaz |
| 332. | Methyl-beta-D-Glucuronlc-NH-p-phenyl-CH₂—CO-D-Cha-Pyr-NH—CH₂-2-(4-am)-thiaz |
| 333. | D-quinic-NH-p-phenyl-CH₂—CO-D-Cha-Pyr-NH—CH₂-2-(4-am)-thiaz |
| 334. | Phenyl-alpha-Iduronic-NH-p-phenyl-CH₂—CO-D-Cha-Pyr-NH—CH₂-2-(4-am)-thiaz |
| 335. | Digalacturonlc-NH-p-phenyl-CH₂—CO-D-Cha-Pyr-NH—CH₂-2-(4-am)-thiaz |
| 336. | Trigalacturonic-NH-p-phenyl-CH₂—CO-D-Cha-Pyr-NH—CH₂-2-(4-am)-thiaz |
| 337. | 3,4,5-Trihydroxy-6-hydroxymethyl-tetrahydropyrany[(2)-CO—NH-p-phenyl-CH₂—CO-D-Cha- |
| | Pyr-NH—CH₂-2-(4-am)-thiaz |
| 338. | 3-Acetamido-4,5-dihydroxy-6-hydroxymethyl-tetrahydropyranyl(2)-CO—NH-p-phenyl-CH₂—CO- |
| | D-Cha-Pyr-NH—CH₂-2-(4-am)-thiaz |
| 339. | D-Galacturo-NH-p-phenyl-CO-D-Cha-Pyr-NH—CH₂-2-(4-am)-thiaz |
| 340. | D-Glucohepto-NH-p-phenyl-CO-D-Cha-Pyr-NH—CH₂-2-(4-am)-thiaz |
| 341. | L-Allo-NH-p-phenyl-CO-D-Cha-Pyr-NH—CH₂-2-(4-am)-thiaz |
| 342. | D-Allo-NH-p-phenyl-CO-D-Cha-Pyr-NH—CH₂-2-(4-am)-thiaz |
| 343. | D Gluco-NH-p-henyl-CO-D-Cha-Pyr-NH—CH₂-2-(4-am)-thiaz |
| 344. | D-Galacto-NH-p-phenyl-CO-D-Cha-Pyr-NH—CH₂-2-(4-am)-thiaz |
| 345. | L-Gluco-NH-p-phenyl-CO-D-Cha-Pyr-NH—CH₂-2-(4-am)-thiaz |
| 346. | L-Manno-NH-p-phenyl-CO-D-Cha-Pyr-NH—CH₂-2-(4-am)-thiaz |
| 347. | D-Manno-NH-p-phenyl-CO-D-Cha-Pyr-NH—CH₂-2-(4-am)-thiaz |
| 348. | D-Cellotrio-NH-p-phenyl-CO-D-Cha-Pyr-NH—CH₂-2-(4-am)-thiaz |
| 349. | D-Cellobio-NH-p-phenyl-CO-D-Cha-Pyr-NH—CH₂-2-(4-am)-thiaz |
| 350. | D-Glucuronic-NH-p-phenyl-CO-D-Cha-Pyr-NH—CH₂-2-(4-am)-thiaz |
| 351. | Arabinic AC-NH-p-phenyl-CO-D-Cha-Pyr-NH—CH₂-2-(4-am)-thiaz |
| 352. | L-lduronic-NH-p-phenyl-CO-D-Cha-Pyr-NH—CH₂-2-(4-am)-thiaz |
| 353. | Gluconic-NH-p-phenyl-CO-D-Cha-Pyr-NH—CH₂-2-(4-am)-thiaz |
| 354. | Heptagluconic-NH-p-phenyl-CO-D-Cha-Pyr-NH—CH₂-2-(4-am)-thiaz |
| 355. | Lactobionic-NH-p-phenyl-CO-D-Cha-Pyr-NH—CH₂-2-(4-am)-thiaz |
| 356. | D-Xylonic-NH-p-phenyl-CO-D-Cha-Pyr-NH—CH₂-2-(4-am)-thiaz |
| 357. | Arabic-NH-p-phenyl-CO-D-Cha-Pyr-NH—CH₂-2-(4-am)-thiaz |
| 358. | Phenyl-beta-D-Glucuronic-NH-p-phenyl-CO-D-Cha-Pyr-NH—CH₂-2-(4-am)-thiaz |
| 359. | Methyl-beta-D-Glucuronic-NH-p-phenyl-CO-D-Cha-Pyr-NH—CH₂-2-(4-am)-thiaz |
| 360. | D-quinlc-NH-p-phenyl-CO-D-Cha-Pyr-NH—CH₂-2-(4-am)-thiaz |
| 361. | Phenyl-alpha-iduronic-NH-p-phenyl-CO-D-Cha-Pyr-NH—CH₂-2-(4-am)-thiaz |
| 362. | Digalacturonic-NH-p-phenyl-CO-D-Cha-Pyr-NH—CH₂-2-(4-am)-thiaz |
| 363. | 3,4,5-Trihydroxy-6-hydroxymethyl-tetrahydropyranyl(2)-CO—NH-p-phenyl-CO-D-Cha-Pyr- |
| | NH—CH₂-2-(4-am)-thiaz |
| 364. | 3-acetamido-4,5-dihydroxy-6-hydroxymethyl-tetrahydropyranyl(2)-CO—NH-p-phenyl-CO-D- |
| | Cha-Pyr-NH—CH₂-2-(4-am)-thiaz |
| 365. | Trlgalacturonic-NH-p-phenyl-CO-D-Cha-Pyr-NH—CH₂-2-(4-am)-thiaz |
| 366. | L-Glycer-D-Chg-Pyr-NH—CH₂-5-(3-am)-thioph |
| 367. | D-Glycer-D-Chg-Pyr-NH—CH₂-5-(3-am)-thioph |
| 368. | L-Erythro-D-Chg-Pyr-NH—CH₂-5-(3-am)-thioph |
| 369. | D-Erythro-D-Chg-Pyr-NH—CH₂-5-(3-am)-thioph |
| 370. | L-Threo-D-Chg-Pyr-NH—CH₂-5-(3-am)-thioph |
| 371. | D-Threo-D-Chg-Pyr-NH—CH₂-5-(3-am)-thioph |
| 372. | L-Arabino-D-Chg-Pyr-NH—CH₂-5-(3-am)-thioph |
| 373. | D-Arabino-D-Chg-Pyr-NH—CH₂-5-(3-am)-thioph |
| 374. | L-Ribo-D-Chg-Pyr-NH—CH₂-5-(3-am)-thioph |
| 375. | D-Rlbo-D-Chg-Pyr-NH—CH₂-5-(3-am)-thioph |
| 376. | 2-Deoxy-L-Ribo-D-Chg-Pyr-NH—CH₂-5-(3-am)-thioph |
| 377. | D-Fuco-D-Chg-Pyr-NH—CH₂-5-(3-am)-thioph |
| 378. | D-Xylo-D-Chg-Pyr-NH—CH₂-5-(3-am)-thioph |
| 379. | L-Xylo-D-Chg-Pyr-NH—CH₂-5-(3-am)-thioph |
| 380. | Cellopentao-D-Chg-Pyr-NH—CH₂-5-(3-am)-thioph |
| 381. | D-Fructo-D-Chg-Pyr-NH—CH₂-5-(3-am)-thioph |
| 382. | Maltotrio-D-Chg-Pyr-NH—CH₂-5-(3-am)-thioph |
| 383. | Maltotetrao-D-Chg-Pyr-NH—CH₂-5-(3-am)-thioph |
| 384. | Glucohepto-D-Chg-Pyr-NH—CH₂-5-(3-am)-thioph |
| 385. | L-Allo-D-Chg-Pyr-NH—CH₂-5-(3-am)-thioph |
| 386. | D-Allo-D-Chg-Pyr-NH—CH₂-5-(3-am)-thioph |
| 387. | L-Gluco-D-Chg-Pyr-NH—CH₂-5-(3-am)-thioph |
| 388. | D-Manno-D-Chg-Pyr-NH—CH₂-5-(3-am)-thioph |
| 389. | L-Manno-D-Chg-Pyr-NH—CH₂-5-(3-am)-thioph |
| 390. | L-Galacto-D-Chg-Pyr-NH—CH₂-5-(3-am)-thioph |
| 391. | Dextro-D-Chg-Pyr-NH—CH₂-5-(3-am)-thioph |
| 392. | L-Lyxo-D-Chg-Pyr-NH—CH₂-5-(3-am)-thioph |
| 393. | D-Lyxo-D-Chg-Pyr-NH—CH₂-5-(3-am)-thioph |
| 394. | D-Lacto-D-Chg-Pyr-NH—CH₂-5-(3-am)-thioph |
| 395. | D-Talo-D-Chg-Pyr-NH—CH₂-5-(3-am)-thioph |
| 396. | L-Talo-D-Chg-Pyr-NH—CH₂-5-(3-am)-thioph |
| 397. | beta-Malto-D-Chg-Pyr-NH—CH₂-5-(3-am)-thioph |
| 398. | L-Fuco-D-Chg-Pyr-NH—CH₂-5-(3-am)-thioph |
| 399. | L-Gulo-D-Chg-Pyr-NH—CH₂-5-(3-am)-thioph |
| 400. | D-Gulo-D-Chg-Pyr-NH—CH₂-5-(3-am)-thioph |
| 401. | L-ldo-D-Chg-Pyr-NH—CH₂-5-(3-am)-thioph |
| 402. | D-Ido-D-Chg-Pyr-NH—CH₂-5-(3-am)-thioph |
| 403. | D-Celotrio-D-Chg-Pyr-NH—CH₂-5-(3-am)-thioph |
| 404. | D-Gatacturonic-D-Chg-Pyr-NH—CH₂-5-(3-am)-thioph |
| 405. | L-Rhamno-D-Chg-Pyr-NH—CH₂-5-(3-am)-thioph |
| 406. | D-Cellotetrao-D-Chg-Pyr-NH—CH₂-5-(3-am)-thioph |
| 407. | Maltopentao-D-Chg-Pyr-NH—CH₂-5-(3-am)-thioph |
| 408. | Xylobio-D-Chg-Pyr-NH—CH₂-5-(3-am)-thioph |
| 409. | D-Lacto-D-Chg-Pyr-NH—CH₂-5-(3-am)-thioph |
| 410. | D-Melibio-D-Chg-Pyr-NH—CH₂-5-(3-am)-thioph |
| 411. | Gentobio-D-Chg-Pyr-NH—CH₂-5-(3-am)-thioph |
| 412. | D-Rhamno-D-Chg-Pyr-NH—CH₂-5-(3-am)-thioph |
| 413. | L-Altro-D-Chg-Pyr-NH—CH₂-5-(3-am)-thioph |
| 414. | D-Galacto-D-Chg-Pyr-NH—CH₂-5-(3-am)-thioph |

### LIST OF ABBREVIATIONS

Abu: 2-aminobutyric acid
AIBN: azobisisobutyronitrile
Ac: acetyl
Acpc: 1-aminocyclopentane-1-carboxylic acid
Achc: 1-aminocyclohexane-1-carboxylic acid
Aib: 2-aminoisobutyric acid
Ala: alanine
b-Ala: beta-alanine (3-aminopropionic acid)
am: amidino
amb: amidinobenzyl
4-amb: 4-amidinobenzyl (p-amidinobenzyl)

### Arg: Arginine

Asp: aspartic acid
Aze: azetidine-2-carboxylic acid
Bn: benzyl
Boc: tert-butyloxycarbonyl
Bu: butyl
Cbz: carbobenzoxy
Cha: cyclohexylalanine
Chea: cycloheptylalanine
Cheg: cycloheptylglycine
Chg: cyclohexylglycine
Cpa: cyclopentylalanine
Cpg: cyclopentylglycine
d: doublet
Dab: 2,4-diaminobutyric acid
Dap: 2,3-diaminopropionic acid
DC: thin-layer chromatography
DCC: dicyclohexylcarbodiimide
Dcha: dicyclohexylamine
DCM: dichloromethane
Dhi-1-COOH: 2,3-dihydro-1H-isoindole-1-carboxylic acid
DMF: dimethylformamide
DIPEA: diisopropylethylamine
EDC: N′-(3-dimethylaminopropyl)-N-ethylcarbodiimide
Et: ethyl
Eq: equivalent
Gly: glycine
Glu: glutamic acid
fur: furan
guan: guanidino
ham: hydroxyamidino
HCha: homocyclohexylalanine, 2-amino-4-cyclohexylbutyric acid
His: histidine
HOBT: hydroxylbenzotriazol
HOSucc: hydroxysuccinimide
HPLC: high-performance liquid chromatography
Hyp: hydroxyproline
Ind-2-COOH: indoline-2-carboxylic acid
iPr: isopropyl
Leu: leucine
Lsg: solution
Lys: lysine
m: multiplet
Me: methyl
MPLC: medium-performance liquid chromatography
MTBE: methyl-tert-butyl ether

### NBS: N-bromosuccinimide

Nva: norvaline
Ohi-2-COOH: octahydroindole-2-carboxylic acid
Ohii-1-COOH: octahydro-isoindole-1-carboxylic acid
Orn: ornithine
Oxaz: oxazole
p-amb: p-amidinobenzyl
Ph: phenyl
Phe: phenylalanine
Phg: phenylglycine
Pic: pipecolic acid
pico: picolyl
PPA: propylphosphonic anhydride
Pro: proline
Py: pyridine
Pyr: 3,4-dehydroproline
q: quartet
RP-18: reversed phase C18
RT: room temperature
s: singlet
Sar: sarcosine (N-methylglycine)
sb: singlet broad
t: triplet
t: tertiary (tert)
tBu: tert-butyl
tert: tertiary (tert)
TBAB: tetrabutylammonium bromide
TEA: triethylamine
TFA: trifluoroacetic acid
TFAA: trifluoroacetic anhydride
thiaz: thiazole
Thz-2-COOH: 1,3-thiazolidine-2-carboxylic acid
Thz-4-COOH: 1,3-thiazolidine-4-carboxylic acid
thioph: thiophene
1-Tic: 1-tetrahydro-isoquinoline carboxylic acid
3-Tic: 3-tetrahydro-isoquinoline carboxylic acid
TOTU: O-(cyanoethoxycarbonylmethylene)amino-1-N,N,N′,N′-tetramethyluronium tetrafluoroboronate(?)
Z: carbobenzoxy

### Experimental Section

The compounds of formula I can be represented by schemes I and II.

The building blocks A-B, D, E, G and K are preferably made separately and used in a suitably protected form (cf scheme I, which illustrates the use of orthogonal protective groups (P or P*) compatible with the synthesis method used.

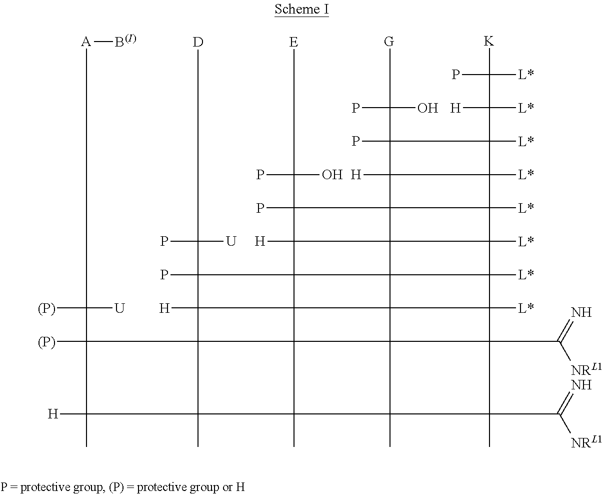

Scheme I describes the linear structure of the molecule I achieved by elimination of protective groups from P-K-L* (L* denotes CONH₂, CSNH₂, CN, C(═NH)NH—COOR*; R* denotes a protective group or polymeric carrier with spacer (solid phase synthesis)), coupling of the amine H-K-L* to the N-protected amino acid P-G-OH to form P-G-K-L*, cleavage of the N-terminal protective group to form H-G-K-L*, coupling to the N-protected amino acid P-E-OH to produce P-E-G-K-L*, re-cleavage of the N-terminal protective group to form H-E-G-K-L* and optionally re-coupling to the N-protected building block β-D-U (U=leaving group) to form β-D-E-G-K-L*, if the end product exhibits a building block D.

If L* is an amide, thioamide or nitrile function at this synthesis stage, it will be converted to the corresponding amidine or hydroxyamidine function, depending on the end product desired. Amidine syntheses for the benzamidine, picolylamidine, thienylamidine, furylamidine, and thiazolylamidine compounds of the structure type I starting from the corresponding carboxylic acid amides, nitriles, carboxythioamides, and hydroxyamidines have been described in a number of patent applications (cf, for example, WO 95/35309, WO 96/178860, WO 96/24609, WO 96/25426, WO 98/06741, and WO 98/09950.

After splitting-off the protective group P to form H-(D)-E-G-K-L* (L* denotes C(═NH)NH, C(═NOH)NH, or (═NH)NH—COOR*; R* denotes a protective group or a polymeric carrier with spacer (solid-phase synthesis), coupling is effected to the optionally protected (P)-A-B-U building block (U=leaving group) or by hydroalkylation with (P)-A-B′-U (U=aldehyde, ketone) to produce (P)-A-B-(D)-E-G-K-L*.

Any protective groups still present are then eliminated. If L* denotes a C(═NH)NH spacer polymer support, these compounds are eliminated from the polymeric support in the final stage, and the active substance is thus liberated.

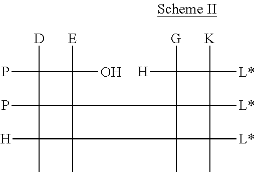

Scheme II describes an alternative route for the preparation of the compounds I by convergent synthesis. The appropriately protected building blocks P-D-E-OH and H-G-K-L* are linked to each other, the resulting intermediate product P-D-E-G-K-L* is converted to P-D-E-G-K-L* (L* denotes C(═NH)NH, C(═NOH)NH, or (═NH)NH—COOR*; R* denotes a protective group or a polymeric support with spacer (solid-phase synthesis), the N-terminal protective group is eliminated, and the resulting product H-D-E-G-K-L* is converted to the end product according to scheme I.

The N-terminal protective groups used are Boc, Cbz, or Fmoc, and C-terminal protective groups are methyl, tert-butyl and benzyl esters. Amidine protective groups for the solid-phase synthesis are preferably Boc, Cbz, and derived groups. If the intermediate products contain olefinic double bonds, then protective groups that are eliminated by hydrogenolysis are unsuitable.

The necessary coupling reactions and the conventional reactions for the provision and removal of protective groups are carried out under standardized conditions used in peptide chemistry (cf M. Bodanszky, A. Bodanszky, “The Practice of Peptide Synthesis”, 2nd Edition, Springer Verlag Heidelberg, 1994).

Boc protective groups are eliminated by means of dioxane/HCl or TFA/DCM, Cbz protective groups by hydrogenolysis or with HF, and Fmoc protective groups with piperidine. Saponification of ester functions is carried out with LiOH in an alcoholic solvent or in dioxane/water. tert-Butyl esters are cleaved with TFA or dioxane/HCl.

The reactions were monitored by DC, in which the following mobile solvents were usually employed:

| | |
|---|---|
| A. DCM/MeOH | 95:5 |
| B. DCM/MeOH | 9:1 |
| C. DCM/MeOH | 8:2 |
| D. DCM/MeOH/HOAc 50% | 40:10:5 |
| E. DCM/MeOH/HOAc 50% | 35:15:5 |

If column separations are mentioned, these separations were carried out over silica gel, for which the aforementioned mobile solvents were used.

Reversed phase HPLC separations were carried out with acetonitrile/water and HOAc buffer.

The starting compounds can be produced by the following methods:

### Building Blocks A-B:

The compounds used as building blocks A-B are for the most part commercially available sugar derivatives. If these compounds have several functional groups, protective groups are introduced at the required sites. If desired, functional groups are converted to reactive groups or leaving groups (eg, carboxylic acids to active esters, mixed anhydrides, etc.), in order to make it possible to effect appropriate chemical linking to the other building blocks. The aldehyde or keto function of sugar derivatives can be directly used for hydroalkylation with the terminal nitrogen of building block D or E.

The synthesis of building blocks D is carried out as follows:

The building blocks D—4-aminocyclohexanoic acid, 4-aminobenzoic acid, 4-aminomethylbenzoic acid, 4-aminomethylphenylacetic acid, and 4-aminophenylacetic acid—are commercially available.

The synthesis of the building blocks E was carried out as follows:

The compounds used as building blocks E—glycine, (D)- or (L)-alanine, (D)- or (L)-valine, (D)-phenylalanine, (D)-cyclohexylalanine, (D)-cycloheptylglycine, D-diphenylalanine, etc. are commercially available as free amino acids or as Boc-protected compounds or as the corresponding methyl esters.

Preparation of cycloheptylglycine and cyclopentylglycine was carried out by reaction of cycloheptanone or cyclopentanone respectively with ethyl isocyanide acetate according to known instructions (H.-J. Prätorius, J. Flossdorf, M. Kula, Chem. Ber. 1985, 108, 3079, or U. Schöllkopf and R. Meyer, Liebigs Ann. Chem. 1977, 1174). Preparation of (D)-dicyclohexylalanine was carried out by hydrogenation after T. J. Tucker et al, J. Med. Chem. 1997, 40., 3687-3693.

The said amino acids were provided by well-known methods with an N-terminal or C-terminal protective group depending on requirements.

Synthesis of the building blocks G was carried out as follows:

The compounds used as building blocks G—(L)-proline, (L)-pipecolinic acid, (L)-4,4-difluoroproline, (L)-3-methylproline, (L)-5-methylproline, (L)-3,4-dehydroproline, (L)-octahydroindole-2-carboxylic acid, (L)-thiazolidine-4-carboxylic acid, and (L)-azetidine carboxylic acid—are commercially available as free amino acids or as Boc-protected compounds or as corresponding methyl esters.

(L)-Methyl thiazolidine-2-carboxylate was prepared after R. L. Johnson, E. E. Smissman, J. Med. Chem. 21, 165 (1978).

Synthesis of the building blocks K was carried out as follows:
p-Cyanobenzylamine

Preparation of this building block was carried out as described in WO 95/35309.

### 3-(6-Cyano)picolylamine

Preparation of this building block was carried out as described in WO 96/25426 or WO 96/24609.

### 5-Aminomethyl-2-cyanothiophen

Preparation of this building block was carried out as described in WO 95/23609.

### 5-Aminomethyl-3-cyanothiophen

Preparation of this building block was carried out starting from 2-formyl-4-cyanothiophen in a manner similar to that described for 2-formyl-5-cyanothiophen (WO 95/23609).

### 2-Aminomethylthiazole-4-thiocarboxamide

Preparation was carried out according to G. Videnov, D. Kaier, C. Kempter and G. Jung, Angew. Chemie (1996) 108, 1604, where the N-Boc-protected compound described in said reference was deprotected with ethereal hydrochloric acid in dichloromethane.

### 5-Aminomethyl-2-cyanofuran

Preparation of this building block was carried out as described in WO 96/17860.

### 5-Aminomethyl-3-cyanofuran

Preparation of this building block was carried out as described in WO 96/17860.

### 5-Aminomethyl-3-methylthiophene-2-carbonitrile

Preparation of this building block was carried out as described in WO 99/37668.

### 5-Aminomethyl-3-chlorothiophene-2-carbonitrile

Preparation of this building block was carried out as described in WO 99/37668.

### 5-Aminomethyl-4-methylthiophene-3-thiocarboxamide

Preparation of this building block was carried out as described in WO 99/37668.

### 5-Aminomethyl-4-chlorothiophene-3-thiocarboxamide

Preparation of this building block was carried out as described in WO 99/37668.

### 2-Aminomethyl-4-cyanothiazole

### a) Boc-2-aminomethylthiazole-4-carboxamide

To a solution of Boc-glycinethioamide (370 g, 1.94 mol) in 3.9 liters of ethanol there was added ethyl bromopyruvate (386 g, 1.98 mol) dropwise at 10° C., and the mixture was stirred over a period of 5 h at from 20° to 25° C. Then 299 mL of 25% strength aqueous ammonia were added.
940 mL of this mixture (equivalent to 19.9% of the total volume) were taken and 380 mL of ethanol were removed therefrom by distillation, after which 908 mL of 25% strength aqueous ammonia were added, and the mixture was stirred for 110 h at from 20° to 25° C. The mixture was cooled to 0° C., and the solids were filtered off and washed twice with water and dried. There were obtained 60.1 g of Boc-protected thiazole carboxamide having an HPLC purity of 97.9 areal %, corresponding to a yield for these two stages of 60.5%.

¹H-NMR (DMSO-d₆, in ppm): 8.16 (s, 1H, NH), 7.86 (t, broad, 1H, NH), 7.71 and 7.59 (2×s, broad, each 1H, NH₂), 4.42 (d, 2H, CH₂), 1.41 (s, 9H, tert-butyl)

### b) 2-Aminomethyl-4-cyanothiazole hydrochloride

Boc-2-aminomethylthiazole 4-carboxamide (75.0 g, 0.29 mol) was suspended in 524 mL of dichloromethane and triethylamine (78.9 g, 0.78 mol) and 79.5 g (0.38 mol) of trifluoroacetic anhydride were added thereto at from −5° to 0° C. Stirring was continued over a period of 1 h, the mixture heated to from 20° to 25° C. and 1190 mL of water added, and the phases were separated. To the organic phase there were added 160 mL of from 5 to 6N isopropanolic hydrochloric acid, and the mixture was heated at boiling temperature over a period of 3 h and then at from 20° to 25° C. overnight with stirring, after which it was cooled to from −5° to 0° C. for 2.5 h prior to removal of the solids by filtering. This solid material was washed with dichloromethane and dried. There were obtained 48.1 g of 2-aminomethyl-4-cyanothiazole having an HPLC purity of 99.4 areal %, which is equivalent to a yield for these two stages of 94.3%.

¹H-NMR (DMSO-d₆, in ppm): 8.98 (s, broad, 2H, NH₂), 8.95 (s, 1 h, Ar—H), 4.50 (s, 2H, CH₂)

### 5-Aminomethyl-3-amidinothiophene bishydrochloride

Synthesis of this compound was carried out starting from 5-aminomethyl-3-cyanothiophene by reaction with (Boc)₂O to form 5-tert-butyl-oxycarbonylaminomethyl-3-cyanothiophene, conversion of the nitrile function to the corresponding thioamide by the addition of hydrogen sulfide, methylation of the thioamide function with iodomethane, reaction with ammonium acetate to produce the corresponding amidine followed by protective group elimination with hydrochloric acid in isopropanol to give 5-aminomethyl-3-amidinothiophene bishydrochloride.

Building blocks for solid-phase synthesis:

### 3-Amidino-5-[N-1-(4,4-dimethyl-2,6-dioxocyclohexylidene)ethyl]aminomethylthiophene hydrochloride

3-Amidino-5-aminomethylthiophene bishydrochloride (1.3 g, 5.7 mmol) was placed in DMF (15 mL), and N,N-diisopropylethylamine (0.884 g, 6.84 mmol) was added. Following stirring for 5 min at room temperature there were added acetyldimedone (1.25 g, 6.84 mmol) and trimethoxymethane (3.02 g, 28.49 mmol). Stirring was continued for 2.5 h at room temperature, after which the DMF was removed in high vacuum and the residue was stirred with DCM (5 mL) and petroleum ether (20 mL). The solvent was decanted from the pale yellow product and the solid matter was dried in vacuo at 40° C. Yield: 1.84 g (5.2 mmol, 91%).

¹H-NMR (400 MHz, [D6]DMSO, 25° C., TMS): delta=0.97 (s, 6H); 2.30 (s, 4H); 2.60 (s, 4H); 4.96 (d, J=7 Hz, 2H); 7.63 (s, 1H); 8.60 (s, 1H); 9.07 (sbr, 2H); 9.37 (sbr, 1H).

### Syntheses of Building Blocks H-G-K-CN:

The synthesis of the H-G-K-CN building block is exemplarily described in WO 95/35309 for prolyl-4-cyanobenzylamide, in WO 98/06740 for 3,4-dehydroprolyl-4-cyanobenzylamide and in WO 98/06741 for 3,4-dehydroprolyl-5-(2-cyano)thienylmethylamide. The preparation of 3,4-dehydroprolyl-5-(3-cyano)thienylmethylamide is similarly carried out by coupling Boc-3,4-dehydroproline to 5-aminomethyl-3-cyanothiophen hydrochloride followed by protective group elimination.

The synthesis of 3,4-dehydroprolyl[2(4-cyano)thiazolmethyl]amide hydrochloride was carried out by coupling Boc-3,4-dehydroproline to 2-aminomethyl-4-cyanothiazole hydrochloride followed by protective group elimination.

### H-E-G-K-C(═NOH)NH₂:

The synthesis of the building block H-E-G-K-C(═NOH)NH₂ is exemplarily described for H-(D)-Cha-Pyr-NH—CH₂-2-(4-ham)thiaz

### a) (Boc)-(D)-cyclohexylalanyl-3,4-dehydroprolyl-[2-(4-cyano)thiazolyl]methylamide

(Boc)-(D)-Cha-OH (21.3 g, 271.4 mmol) and H-Pyr-NH—CH₂-2(4-CN)-thiaz hydrochloride (21.3 g, 270.7 mmol) were suspended in dichloromethane (750 mL) and to the suspension there was added ethyldiisopropylamine (50.84 g, 67.3 mL, 393 4 mmol), which gave a clear, slightly reddish solution. The reaction mixture was cooled to ca 10° C., and a 50% strength solution of propylphosphonic anhydride in ethyl acetate (78.6 mL, 102.3 mmol) was added dropwise. Following stirring overnight at RT, the mixture was concentrated in vacuo, the residue taken up in water and the mixture stirred for 30 min to effect hydrolysis of the excess propylphosphonic anhydride. The acid solution was then extracted 3 times with ethyl acetate and once with dichloromethane, the organic phases being washed with water, dried, and evaporated in vacuo in a rotary evaporator. The two residues were combined, dissolved in dichloromethane and precipitated with n-pentane. This procedure was repeated and 33.4 g of (Boc)-(D)-Cha-Pyr-NH—CH₂-2(4-CN)thiaz (yield 87%) were obtained as white solid.

### b) (Boc)-(D)-cyclohexylalanyl-3,4-dehydroprolyl-[2-(4-hydroxamidino)thiazolyl]methylamide

(Boc)-(D)-Cha-Pyr-NH—CH₂-2-(4-CN)-thiaz (26.3 g, 53.9 mmol) was dissolved in methanol (390 mL), to the solution there was added hydroxylamine hydrochloride (9.37 g, 134.8 mmol), and to this suspension diisopropylethylamine (69.7 g, 91.7 mL, 539.4 mmol) was slowly added dropwise, with cooling (water bath). Following agitation at room temperature over a period of 3 h, the reaction solution was evaporated in vacuo in a rotary evaporator, the residue taken up in ethyl acetate/water, and the aqueous phase was set to pH 3 with 2N hydrochloric acid and extracted 3 times with ethyl acetate and once with dichloromethane. The organic phases were washed a number of times with water, dried over magnesium sulphate and evaporated in vacuo in a rotary evaporator. The two residues were combined and stirred with n-pentane to give 26.8 g of (Boc)-(D)-Cha-Pyr-NH—CH₂-2(4-ham)-thiaz (yield 95%) as a white solid.

### c) (D)-cyclohexylalanyl-3,4-dehydroprolyl-[2-(-4-hydroxamidino)thiazolyl]methylamide

(Boc)-(D)-Cha-Pyr-NH—CH₂-2(4-ham)-thiaz (5.0 g, 9.6 mmol) was dissolved in a mixture of isopropanol (50 mL) and dichloromethane (50 mL) and to the solution there was added HCl in dioxane (4M solution, 24 mL, 96 mmol) and stirring was continued for 3 h at room temperature. As starting material was still present, HCl in dioxane (4M solution, 12 mL, 48 mmol) was again added and the mixture stirred at room temperature overnight. The reaction mixture was evaporated in vacuo in a rotary evaporator, and co-distilled a number of times with ether and dichloromethane to remove adhering hydrochloric acid. The residue was dissolved in a little methanol and precipitated with a large quantity of ether. There were obtained 4.3 g of H-(D)-Cha-Pyr-NH—CH₂-2(4-ham)thiaz hydrochloride (yield 98%).

### H-E-G-K-C(═NH)NH₂:

The synthesis of the H-E-G-K-C(═NH)NH₂ building block is exemplarily described for H-(D)-Cha-Pyr-NH—CH₂-2(4-am)thiaz.

### a) (Boc)-(D)-cyclohexylalanyl-3,4-dehydroprolyl-[2-(4-amidino)thiazolyl]methylamide

(Boc)-(D)-Cha-Pyr-NH—CH₂-2-(4-CN)-thiaz (27.0 g, 55.4 mmol) and N-acetyl-L-cysteine (9.9 g, 60.9 mmol) were dissolved in methanol (270 mL), heated under reflux, while ammonia was introduced over a period of 8 h. Since the reaction was still non-quantitative after DC checking, N-acetyl-L-cysteine (2.0 g, 12.0 mmol) was again added and the mixture heated under reflux for a further 8 h with introduction of ammonia. The reaction mixture was then concentrated in vacuo, and the residue was successively stirred in ether and dichloromethane/ether 9:1. The resulting crude product (Boc)-(D)-Cha-Pyr-NH—CH₂-2(4-am)thiaz, which still contained N-acetyl-L-cysteine, was used without further purification in the next stage.

### b) (D)-cyclohexylalanyl-3,4-dehydroprolyl-[2(4-amidino)thiazolyl]methylamide

(Boc)-(D)-Cha-Pyr-NH—CH₂-2(4-am)thiaz (crude product, see above) was dissolved in a mixture of methanol (20 mL) and dichloromethane (400 mL), and to the solution there was added HCl in dioxane (4M solution, 205 mL, 822 mmol) and stirring was continued overnight at room temperature.
As starting material was still present, HCl in dioxane was again added and stirring carried out overnight at room temperature. The reaction mixture was evaporated in vacuo in a rotary evaporator, and co-distilled a number of times with ether and dichloromethane to remove adhering hydrochloric acid. The residue was taken up in water and extracted 20 times with dichloromethane to remove N-acetyl-L-cysteine, and the aqueous phase was then lyophilized. The lyophilized matter was stirred out from ether to give 31.8 g of H-(D)-Cha-Pyr-NH—CH₂-2(4-am)thiaz dihydrochloride (yield over 2 stages: 81%).

The preparation of the building block H-E-G-K-C(═NH)NH₂H-(D)-Chg-Aze-NH 4-amb is described in WO 94/29336 Example 55. H-(D)-Chg-Pyr-NH—CH₂5-(3-am)-thioph was synthesized in a similar manner to that used for H-(D)-Cha-Pyr-NH—CH₂-2-(4-am)-thiaz, the formation of amidine being effected using the corresponding nitrile precursor Boc-(D)-Chg-Pyr-NH—CH₂-5-(3-CN)-thioph as described in WO 9806741 Example 1 via intermediate stages Boc-(D)-Chg-Pyr-NH—CH₂-5-(3CSNH₂)-thioph and Boc-(D)-Chg-Pyr-NH—CH₂-5-(3—C(═NH)S—CH₃)-thioph.

### Example 1

### (D)-Arabino-(D)-Cha-Pyr-NH—CH₂-2-(4-am)-thiaz×CH₃COOH

H-(D)-Cha-Pyr-NH—CH₂-2-(4-am)-thiaz dihydrochloride (2.0 g, 4.19 mmol) was dissolved in methanol (30 mL), and to the solution there were added D-(−)-arabinose (0.63 g, 4.19 mmol) and molecular sieve (4 Angstrom). The mixture was stirred over a period of 1 h at room temperature and sodium cyanoborohydride was then added portionwise, during which operation slight generation of gas occurred. Following stirring overnight at room temperature, the molecular sieve was filtered off in vacuo, the filtrate concentrated in vacuo and the residue stirred in acetone. The crude product filtered off in vacuo was purified by means of MPLC(RP-18 column, acetonitrile/watter/glacial acetic acid) and then lyophilized to give 840 mg of (D)-Arabino-(D)-Cha-Pyr-NH—CH₂-2-(4-am)thiaz×CH₂COOH as a white solid (yield 34%).

ESI-MS: M+H⁺: 539

### Example 2

### (L)-Arabino-(D)-Cha-Pyr-NH—CH₂-2-(4-am)-thiaz×CH₃COOH

This compound was synthesized in a manner similar to that described in Example 1 but starting from L-(+)-arabinose.

ESI-MS: M+H⁺: 539

### Example 3

### (D)-Erythro-(D)-Cha-Pyr-NH—CH₂-2-(4-am)-thiaz×CH₃COOH

This compound was synthesized in a manner similar to that described in Example 1 but starting from D-(+)-erythrose.

ESI-MS: M+H⁺: 509

### Example 4

### (L)-Erythro-(D)-Cha-Pyr-NH—CH₂-2-(4-am)-thiaz×CH₃COOH

This compound was synthesized in a manner similar to that described in Example 1 but starting from L-(+)-erythrose.

ESI-MS: M+H⁺: 509

### Example 5

### (D)-Glycer-(D)-Cha-Pyr-NH—CH₂-2-(4-am)-thiaz×CH₃COOH

This compound was synthesized in a manner similar to that described in Example 1 but starting from D-(+)-glycerinaldehyde.

ESI-MS: M+H⁺: 479

### Example 6

### (L)-Glycer-(D)-Cha-Pyr-NH—CH₂-2-(4-am)-thiaz×CI₃COOH

This compound was synthesized in a manner similar to that described in Example 1 but starting from L-(+)-glycerinaldehyde.

ESI-MS: M+H⁺: 479

### Example 7

### (L)-Rhamno-(D)-Cha-Pyr-NH—CH₂-2-(4-am)-thiaz×HCl

This compound was synthesized in a manner similar to that described in Example 1 but starting from L-rhamnose.

L-rhamnnose (0.82 g, 5 mmol) was dissolved in water (20 mL) at room temperature and H-(D)-Cha-Pyr-NH—CH₂-2(4-am)thiaz dihydrochloride (2.4 g, 5 mmol) was stirred in. The clear solution became viscous after 20 min. Sodium cyanoborohydride was added portionwise in an equimolar amount over a period of 4 h to give a white precipitate, which dissolved on the addition of ethanol (2 mL). 5 mL of 1M HCl set the pH to 3 and solid was precipitated 3 times with 300 mL of acetone each time. The solid was removed by centrifugation and dissolved in water (100 mL). Following lyophilization there were obtained 2.6 g of (L)Rhamno-(D)-Cha-Pyr-NH—CH₂-2-(4-am)-thiaz xHCl as a white powder.

### Example 8

### (D)-Melibio-(D)-Cha-Pyr-NH—CH₂-2-(4-am)-thiaz×HCl

This compound was synthesized in a manner similar to that described in Example 7 but starting from D-melibiose.

D-melibiose (1.8 g, 5 mmol) was dissolved in water (20 mL) at room temperature and H-(D)-Cha-Pyr-NH—CH₂-2-(4-am)-thiaz dihydrochloride (2.4 g, 5 mmol) was stirred in. The clear pale yellow solution became viscous after 20 min. An equimolar amount of sodium cyanoborohydride was added portionwise over a period of 4 h. There was obtained a white solid precipitate, to which 2 mL of ethanol were added to give a clear solution. The pH was set to pH 5 with 5 mL of 1M HCl and precipitation was effected 3 times with 300 mL of acetone each time. Following centrifugation, the sediment obtained was taken up in 100 mL of water and the solution lyophilized. Yield: 3.2 g of (D)-Melibio-(D)-Cha-PyrNH—CH₂-2-(4-am)-thiaz×HCl.

### Example 9

### (D)-Gluco-(D)-Chg-Pyr-NH—CH₂-5-(3-am)-thioph×HCl

This compound was synthesized in a manner similar to that described in Example 7 but starting from D-glucose.

D-glucose (1.0 g, 5.6 mmol) was dissolved in 20 mL of water at room temperature and H-(D)-Chg-Pyr-NH—CH₂-5-(3-am)-thioph dihydrochloride (3.0 g, 6.5 mmol) was stirred in. The clear solution became viscous after 10 min. An equimolar amount of sodium cyanoborohydride was added portionwise over a period of 4 h to give a white precipitate. After cooling in an ice bath with 3×5 mL of H2O the mixture were shaken and the sediment was taken up in 20 mL of H₂O and the pH set to pH 5.0 with ca 5 mL of 0.1 M NaOH. 1st precipitation using 300 mL of acetone. 2nd precipitation: the sediment was taken up in 30 mL of H₂O and 300 mL of acetone were added. The sediment was dissolved in H₂O and neutralized with 2 mL of 1M HCl; the solution was then lyophilized. Yield: 1.52 g (D)-Gluco-(D)-Chg-Pyr-NH—CH₂-5-(3-am)-thioph×HCl als weiβes Pulver.

### Example 10

### Maltohexao-(D)-Chg-Pyr-NH—CH₂-5-(3-am)-thioph×HCl

This compound was synthesized in a manner similar to that described in Example 7 but starting from maltohexaose.

Maltohexaose (2 g, 2 mmol) was dissolved in water (20 mL) at room temperature and H-(D)-Chg-Pyr-NH—CH₂-5-(3-am)-thioph dihydrochloride (0.92 g, 2 mmol) was stirred in. The clear solution became viscous after 10 min; an equimolar amount of sodium cyanoborohydride was added portionwise over a period of 4 h; after cooling in an ice bath, precipitation was effected with 8 volumes of ethanol. The sediment was reprecipitated with 300 mL of ethanol. The sediment was dissolved in water and the solution lyophilized.

### Example 11

### (D)-Cellobio-(D)-Chg-Pyr-NH—CH₂-5-(3-am)-thioph×HCl

This compound was synthesized in a manner similar to that described in Example 7 but starting from cellobiose.

Cellobiose (2 g, 6 mmol) was stirred into water (20 mL) at 50° C. and H-(D)-Chg-Pyr-NH—CH₂-5-(3-am)-thioph dihydrochloride (2.8 g, 6 mmol) added. The turbid solution became viscous as an equimolar amount of sodium cyanoborohydride was added portionwise over a period of 4 h. Stirring was continued for approximately one hour at 50° C. Approximately 10 mL of 1M HCl were added to set the pH to 3. Precipitation was then effected twice with 300 mL of acetone. Following cooling in an ice bath, the sediment was taken up in 60 mL of water and reprecipitated with 600 mL of acetone. The sediment was dissolved in water and the solution lyophilized. Yield: 4.4 g (D)-Cello-bio-(D)-Chg-Pyr-NH—CH₂-5(3-am)-thioph×HCl.

### Example 12

### (D)-Glucuronic-(D)-Chg-Pyr-NH—CH₂-5-(3-am)-thioph

This compound was synthesized in a manner similar to that described in Example 7 but starting from the sodium salt of D-glucuronic acid.

The sodium salt of D-glucuronic acid×H2O (1.4 g, 6 mmol) was dissolved in water (20 mL) at room temperature and H-(D)-Chg-Pyr-NH—CH₂-5-(3-am)thioph dihydrochloride (2.8 g, 6 mmol) was stirred in at room temperature. The clear solution turned pale yellow after 10 min. An equimolar amount of 330 mg of sodium cyanoborohydride was added portionwise over a period of 4 h to give a solid, compact precipitate. 4 mL of 0.1 M NaOH were added and the supernatant was decanted off and the precipitate stirred up in acetone. The sediment was taken up in 40 mL of H₂O and 3 mL of 1M HCl were added to give a pH of 4. The compound passed into solution. Precipitation was effected with 400 mL of acetone. The sediment was then dissolved in water and the solution lyophilized. Yield: 3.1 g (D)-Glucuronic-(D)-Chg-Pyr-NH—CH₂-5(3-am)-thioph.

### Example 13

### (D)-Gluco-(D)-Chg-Aze-NH-4-amb×HCl

This compound was synthesized in a manner similar to that described in Example 7 but starting from D-glucose.

D-glucose (2.5 g, 14 mmol) was dissolved in water (40 mL) at room temperature and H-(D)-Chg-Aze-NH-4-amb (WO 94/29336 Example 55; 6.8 g; 15.4 mmol) was stirred in. An equimolar amount of sodium cyanoborohydride was added portionwise over a period of 4 h and the mixture was then stirred overnight. There was obtained a greasy, viscous emulsion. 50 mL of water were added, after which ethanol was added until the solution became clear.

The pH was adjusted to 4.0 with ca 15 mL of 0.1M HCl. 1st precipitation using 600 mL of acetone. 2nd precipitation: the sediment was taken up in 50 mL of water and 600 mL of acetone were added; the sediment was redissolved in water and the solution lyophilized. Yield: 7.8 g (D)-Gluco-(D)-Chg-Aze-NH-4-amb×HCl.

### Example 14

### Malto-(D)-Chg-Aze-NH-4-amb×HCl

This compound was synthesized in a manner similar to that described in Example 7 but starting from maltose.

Maltose×H₂O (5 g, 14 mmol) was dissolved in 40 mL of water at room temperature and H-Chg-Aze-NH-4-amb (6.8 g; 15.4 mmol) was stirred in. There followed a portionwise addition of an equimolar amount of sodium cyanoborohydride over a period of 4 h. The initially clear, viscous solution slowly changed to a greasy, viscous emulsion. 50 mL of water were added followed by ca 15 mL 0.1 M HCl to give a pH of 4.0. 1st precipitation using 600 mL of acetone. 2nd precipitation: the sediment was taken up in 50 mL of water and 600 mL of acetone were added; the sediment was redissolved in water and the solution lyophilized. Yield: 10.1 g Malto-(D)-Chg-Aze-NH-4-amb×HCl.

### Example 15

### (L)-Erythro-(D)-Cha-Pyr-NH—CH₂-2-(4-ham)-thiaz×CH₃COOH

This compound was synthesized in a manner similar to that described in Example 1 but starting from L-(+)-erythrose and H-(D)-Cha-Pyr-NH—CH₂-2-(4-ham)thiaz.

ESI-MS: M+H⁺: 525

### Example 16

### (L)-Arabino-(D)-Cha-Pyr-NH—CH₂-2-(4-ham)-thiaz×CH₃COOH

This compound was synthesized in a manner similar to that described in Example 1 but starting from L-(+)-arabinose and H-(D)-Cha-Pyr-NH—CH₂-2-(4-ham)thiaz.

ESI-MS: M+H⁺: 555

### Example 17

### Malto-(D)-Cha-Pyr-NH—CH₂-2-(4-ham)-thiaz

This compound was synthesized in a manner similar to that described in Example 1 but starting from maltose.

H-(D)-Cha-Pyr-NH—CH₂-2-(4-ham)-thiaz Maltose×H2O (2.2 g, 6 mmol) was dissolved in 40 mL of water and 60 mL of ethanol at room temperature and H-(D)-Cha-Pyr-NH—CH₂-2-(4-ham)-thiaz (2.8 g, 6.6 mmol) was stirred in. The portionwise addition of an equimolar amount of sodium cyanoborohydride over a period of 8 h gave a highly viscous, clear, brownish solution. 1st precipitation using 500 mL, of acetone. The sediment was dissolved in 50 mL of water and set to pH 7.5 with 0.1 M of HCl followed by precipitation with 500 mL of acetone. The sediment was dissolved in 100 mL of water and the solution lyophilized. Yield: 3.6 g Malto-(D)-Cha-Pyr-NH—CH₂-2-(4-ham)thiaz.

For the following compounds, the thrombin time was determined according to Example A:

| Example No. | Thrombin time EC₁₀₀ [mol/L] |
|---|---|
| 10 | 2.4E−08 |
| 12 | 1.4E−08 |
| 9 | 1.5E−08 |
| 11 | 2.1E−08 |
| 14 | 2.1E−08 |
| 13 | 2.1E−08 |
| 8 | 1.64E−08 |
| 7 | 9.68E−09 |
| 2 | 1.4E−08 |

1. A compound of the general formula (I)
A-B-D-E-G-K-L (I),
in which
A stands for H, CH₃, H-(R^{A1})i^{A}
in which
R^{A1} denotes 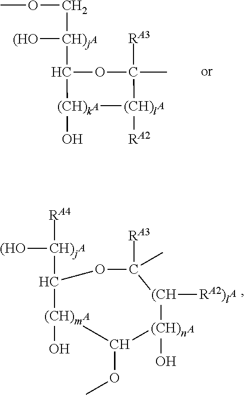
in which
R^{A2} denotes H, NH₂, NH—COCH₃, F, or NHCHO,
R^{A3} denotes H, or CH₂OH,
R^{A4} denotes H, CH₃, or COOH,
ᵢA is 1 to 20,
ⱼA is 0, 1, or 2,
ₖA is 2 or 3,
ₗA is 0 or 1,
ₘA is 0, 1, or 2,
ₙA is 0, 1, or 2,
the groups R^{A1} being the same or different when ᵢA is greater than 1,
B denotes 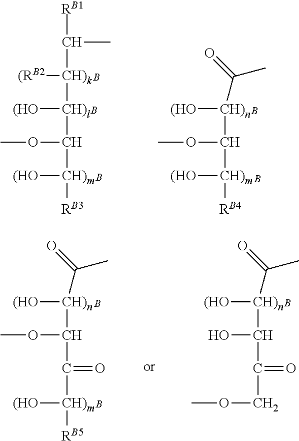
A-B stands for 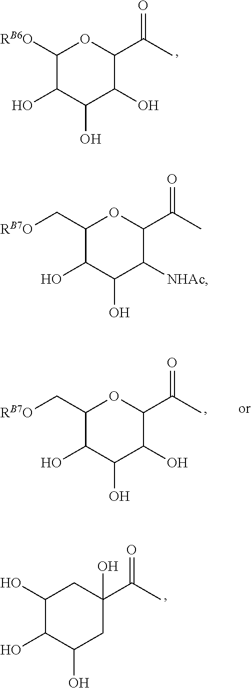
or for a neuraminic acid radical or N-acetylneuraminic acid radical bonded through the carboxyl function,
in which
R^{B1} denotes H, CH₂OH, or C₁₋₄ alkyl,
R^{B2} denotes H, NH₂, NH—COCH₃, F, or NHCHO,
R^{B3} denotes H, C₁₋₄ alkyl, CH₂—O—(C₁₋₄ alkyl), COOH, F, NH—COCH₃, or CONH₂,
R^{B4} denotes H, C₁₋₄ alkyl, CH₂—O—(C₁₋₄ alkyl), COOH, or CHO, in which latter case intramolecular acetal formation may take place,
R^{B5} denotes H, C₁₋₄ alkyl, CH₂—O—(C₁₋₄ alkyl), or COOH,
ₖB is 0 or 1,
ₗB is 0, 1, 2, or 3 (ₗB≠0 when A=R^{B1}=R^{B3}=H, ₘB=ₖB=0 and D is a bond),
ₘB is 0, 1, 2, 3, or 4,
ₙB is 0, 1, 2, or 3,
R^{B6} denotes C₁₋₄ alkyl, phenyl, or benzyl, and
R^{B7} denotes H, C₁₄ alkyl, phenyl, or benzyl,
D stands for a bond or for 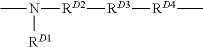
in which
R^{D1} denotes H or C₁₋₄ alkyl,
R^{D2} denotes a bond or C₁₋₄ alkyl,
R^{D3} denotes 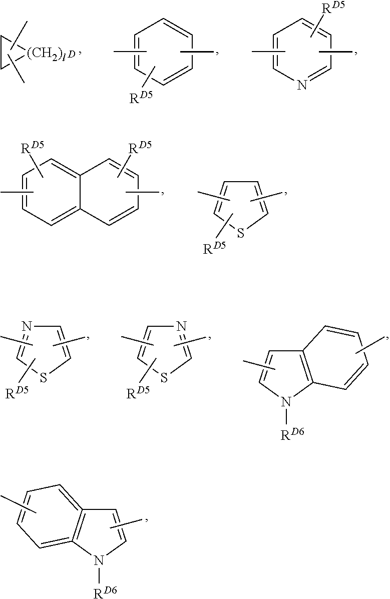
in which
ₗD is 1, 2, 3, 4, 5, or 6,
R^{D5} denotes H, C₁₋₄ alkyl, or Cl, and
R^{D6} denotes H or CH₃,
and in which a further aromatic or aliphatic ring can be condensed onto the ring systems defined for R^{D3},
R^{D4} denotes a bond, C₁₋₄ alkyl, CO, SO₂, or —CH₂—CO,
E stands for 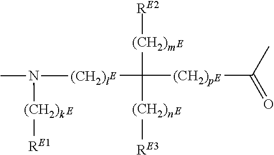
in which
ₖE is 0, 1, or 2,
ₗE is 0, 1, or 2,
ₘE is 0, 1, 2, or 3,
ₙE is 0, 1, or 2,
ₚE is 0, 1, or 2,
R^{E1} denotes H, C₁₋₆ alkyl, C₃₋₈ cycloalkyl, aryl, heteroaryl, C₃₋₈ cycloalkyl having a phenyl ring condensed thereto, which groups may carry up to three identical or different substituents selected from the group consisting of C₁₋₆ alkyl, OH, O—C₁₋₆ alkyl, F, Cl, and Br,
R^{E1} may also denote R^{E4}OCO—CH₂— (where R^{E4} denotes H, C₁₋₁₂ alkyl, or C₁₋₃ alkylaryl),
R^{E2} denotes H, C₁₋₆ alkyl, C₃₋₈ cycloalkyl, aryl, heteroaryl, indolyl, tetrahydropyranyl, tetrahydrothiopyranyl, diphenylmethyl, dicyclohexylmethyl, C₃₋₈ cycloalkyl having a phenyl ring condensed thereto, which groups may carry up to three identical or different substituents selected from the group consisting of C₁₋₆ alkyl, OH, O—(C₁₋₆ alkyl), F, Cl, and Br, and may also denote CH(CH₃)OH or CH(CF₃)₂,
R^{E3} denotes H, C₁₋₆ alkyl, C₃₋₄ cycloalkyl, aryl, heteroaryl, C₃₋₈ cycloalkyl having a phenyl ring condensed thereto, which groups may carry up to three identical or different substituents selected from the group consisting of C₁₋₆ alkyl, OH, O—(Cl₁₋₆ alkyl), F, Cl, and Br,
the groups defined for R^{E1} and R^{E2} may be interconnected through a bond, the
groups defined for R^{E2} and R^{E3} may also be interconnected through a bond,
R^{E2} may also denote COR^{E5} (where R^{E5} denotes OH, O—(C₁₋₆ alkyl), or O—(C₁₋₃ alkylaryl)), CONR^{E6}R^{E7} (where R^{E6} and R^{E7} denote H, C₁₋₆ alkyl, or C₀₋₃ alkylaryl), or NR^{E6}R^{E7},
E may also stand for D-Asp, D-Glu, D-Lys, D-Orn, D-His, D-Dab, D-Dap, or D-Arg,
G stands for 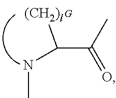
where ₗG is 2, 3, 4, or 5, and one of the CH₂ groups in the ring is replaceable by O, S, NH, N(C₁₋₃ alkyl), CHOH, CHO(C₁₋₃ alkyl), C(C₁₋₃ alkyl)₂, CH(C₁₋₃ alkyl), CHF, CHCl, or CF₂, 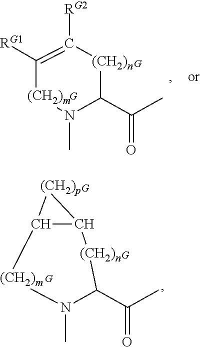
in which
ₘG is 0, 1, or 2,
ₙG is 0, 1, or 2,
ₚG is 0, 1, 2, 3, or 4,
R^{G1} denotes H, C₁₋₆ alkyl, or aryl,
R^{G2} denotes H, C₁₋₆ alkyl, or aryl,
and R^{G1} and R^{G2} may together form a —CH═CH—CH═CH— chain,
G may also stand for 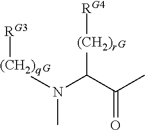
in which
_{q}G is 0, 1, or 2,
ᵣG is 0, 1, or 2,
R^{G3} denotes H, C₁₋₆ alkyl, C₃₋₈ cycloalkyl, or aryl,
R^{G4} denotes H, C₁₋₆ alkyl, C₃₋₈ cycloalkyl, or aryl (particularly phenyl or naphthyl),
K stands for
NH—(CH₂)ₙK-Q^{K}
in which
ₙK is 0, 1, 2, or 3,
Q^{K} denotes C₂₋₆ alkyl, whilst up to two CH₂ groups may be replaced by O or S,
Q^{K} also denotes 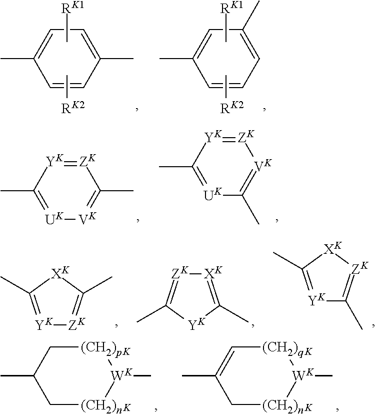
in which
R^{K1} denotes H, C₁₋₃ alkyl, OH, O—C(₁₋₃ alkyl), F, Cl, or Br,
R^{K2} denotes H, C₁₋₃ alkyl, O—(C₁₋₃ alkyl), F, Cl, or Br,
X^{K} denotes O, S, NH, N—(C₁₋₆ alkyl),
Y^{K} denotes ═CH—, 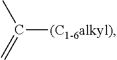
═N—, or 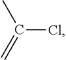
Z^{K} denotes ═CH—, 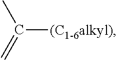
═N—, or 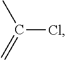
U^{K} denotes ═CH—, 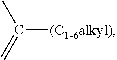
═N—, or 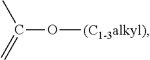
V^{K} denotes ═CH—, 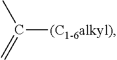
═N—, or 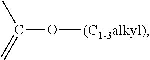
W^{K} denotes 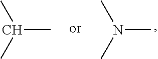
but in the latter case L may not be a guanidine group,
ₙK is 0, 1, or 2,
ₚK is 0, 1, or 2,
_{q}K is 1 or 2,
L stands for 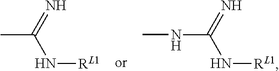
in which
R^{L1} denotes H, OH, O—(C₁₋₆ alkyl), O—(CH₂)₀₋₃-phenyl, CO—(C₁₋₆ alkyl), CO₂—(C₁₋₆ alkyl), or CO₂—(C₁₋₃ alkylaryl),
and the tautomers thereof, stereoisomers thereof, salts thereof with pharmacologically acceptable acids or bases, and the prodrugs thereof.

2. A compound of the general formula (I)
A-B-D-E-G-K-L (I),
in which
A stands for H or H—(R^{A1})i^{A}
in which
R^{A1} denotes 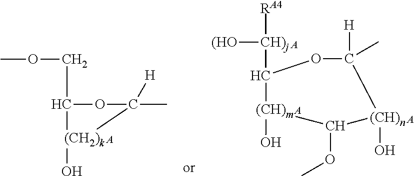
in which
R^{A4} denotes H, CH₃, or COOH,
ᵢA is 1 to 6,
ⱼA is 0, 1, or 2,
ₖA is 2 or 3,
ₘA is 0, 1, or 2,
ₙA is 0, 1, or 2,
the groups R^{A1} being the same or different when ᵢA is greater than 1;
B denotes 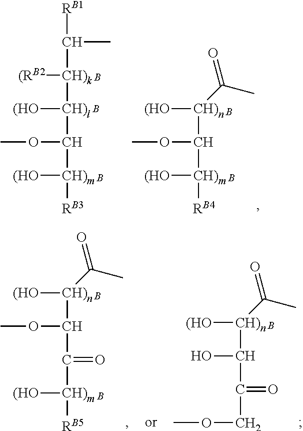
A-B stands for 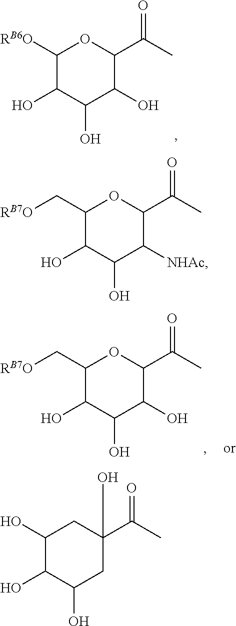
in which
R^{B1} denotes H or CH₂OH,
R^{B2} denotes H, NH₂, NH—COCH₃, or F,
R^{B3} denotes H, CH₃, CH₂—O—(C₁₋₄ alkyl), or COOH,
R^{B4} denotes H, C₁₋₄ alkyl, CH₂—O—(C₁₋₄ alkyl), COOH, or CHO, in which latter case intramolecular acetal formation may take place,
R^{B5} denotes H, CH₃, CH₂—O—(C₁₋₄ alkyl), or COOH,
ₖB is 0 or 1,
ₗB is 0, 1, 2, or 3 (ₗB≠0 when A=R^{B1}=R^{B3}=H, ₘB=ₖB=0, and D is a bond),
ₘB is 0, 1, 2, or 3,
ₙB is 0, 1, 2, or 3,
R^{B6} denotes C₁₋₄ alkyl, phenyl, or benzyl, and
R^{B7} denotes H, C₁₋₄ alkyl, phenyl, or benzyl,
D stands for a bond or for 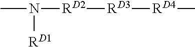
in which
R^{D1} denotes H or C₁₋₄ alkyl,
R^{D2} denotes a bond or C₁₋₄ alkyl,
R^{D3} denotes 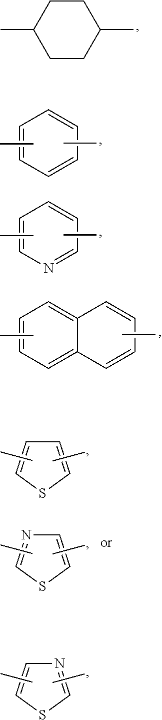
R^{D4} denotes a bond, C₁₋₄ alkyl, CO, SO₂, or —CH₂—CO,
E stands for 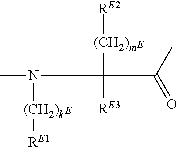
in which
ₖE is 0, 1, or 2,
ₘE is 0, 1, 2, or 3,
R^{E1} denotes H, C₁₋₆ alkyl, or C₃₋₈ cycloalkyl, which groups may carry up to three identical or different substituents selected from the group consisting of C₁₋₆ alkyl, OH, and O—C₁₋₆ alkyl,
R^{E2} denotes H, C₁₋₆ alkyl, C₃₋₈ cycloalkyl, aryl, heteroaryl, tetrahydropyranyl, diphenylmethyl, or dicyclohexylmethyl, which groups may carry up to three identical or different substituents selected from the group consisting of C₁₋₆ alkyl, OH, O—(C₁₋₆ alkyl), F, Cl, and Br, and may also denote CH(CF₃)₂;
R^{E3} denotes H, C₁₋₆ alkyl, or C₃₋₈ cycloalkyl,
R^{E2} may also denote COR^{E5} (where R^{E5} denotes OH, O—C₁₋₆ alkyl, or O—(C₁₋₃ alkylaryl)), CONR^{E6}R^{E7} (where R^{E6} and R^{E7} denote H, C₁₋₆ alkyl, or C₀₋₃ alkylaryl respectively), or NR^{E6}R^{E7};
E may also stand for D-Asp, D-Glu, D-Lys, D-Orn, D-His, D-Dab, D-Dap, or D-Arg;
G stands for 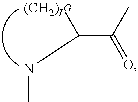
where ₗG is 2, 3, or 4, and one of the CH₂ groups in the ring is replaceable by O, S, NH, N(C₁₋₃ alkyl), CHOH, or CHO(C₁₋₃ alkyl); 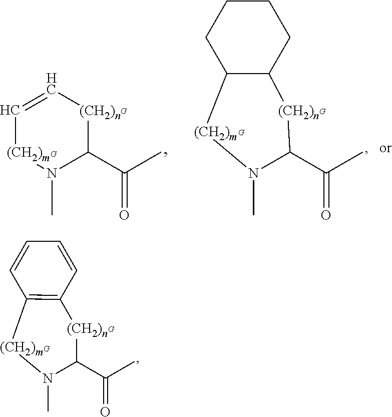
in which
ₘG is 0, 1, or 2;
ₙG is 0, or 1;
K stands for
NH—(CH₂)ₙK-Q^{K}
in which
K is 1 or 2,
Q^{K} denotes 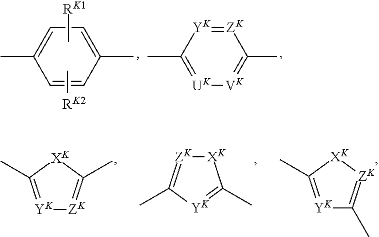
in which
R^{K1} denotes H, C₁₋₃ alkyl, OH, O—(C₁₋₃ alkyl), F, Cl, or Br,
R^{K2} denotes H, C₁₋₃ alkyl, O—(C₁₋₃ alkyl), F, Cl, or Br,
X^{K} denotes O, S, NH, N—(C₁ alkyl),
Y^{K} denotes ═CH—, 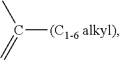
═N—, or 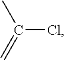
Z^{K} denotes ═CH—, 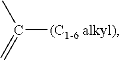
═N—, or 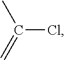
U^{K} denotes ═CH—, 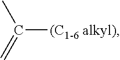
═N—, or 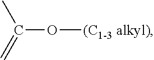
and
L stands for 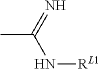
in which
R^{L1} denotes H, OH, O—(C₁₋₆ alkyl), or CO₂—(C₁₋₆ alkyl),
and the tautomers thereof, stereoisomers thereof, salts thereof with pharmacologically acceptable acids or bases, and the prodrugs thereof.

3. A compound of the general formula (I)
A-B-D-E-G-K-L (I),
in which
A stands for H or H—(R^{A1})i^{A}
in which
R^{A1} denotes 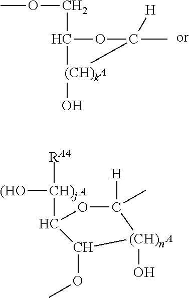
in which
R^{A4} denotes H, or COOH,
ᵢA is 1 to 6,
ⱼA is 0 or 1,
ₖA is 2 or 3,
ₙA is 1 or 2,
the groups R^{A1} being the same or different when ᵢA is greater than 1;
B denotes 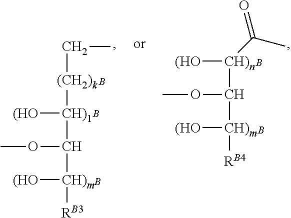
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3,
ₙB is 1, 2, or 3,
D stands for a bond
E stands for 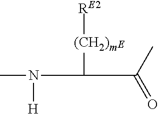
in which
ₘE is 0 or 1,
R^{E2} denotes H, C₁₋₆ alkyl, C₃₋₈ cycloalkyl, aryl, phenyl, diphenylmethyl, or dicyclohexylmethyl, which groups may carry up to three identical or different substituents selected from the group consisting of C₁₋₄ alkyl, OH, O—CH₃, F, and Cl;
G stands for 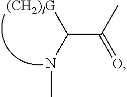
where ₗG is 2, 3, or 4 and one of the CH₂ groups in the ring is replaceable by O, S, NH, or N(C₁₋₃ alkyl), 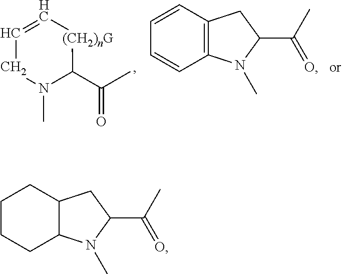
in which
ₙG is 0 or 1;
K stands for
NH—CH₂-Q^{K}
in which
Q^{K} denotes 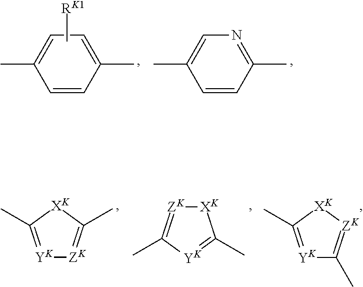
in which
R^{K1} denotes H, CH₃, OH, O—CH₃, F, or Cl,
X^{K} denotes O, S, NH, N—CH₃,
Y^{K} denotes ═CH—, 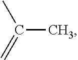
or ═N—,
Z^{K} denotes ═CH—, 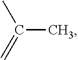
or ═N—; and
L stands for 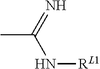
in which
R^{L1} denotes H, OH, or CO₂—(C₁₋₆ alkyl),
and the tautomers thereof, stereoisomers thereof, salts thereof with pharmacologically acceptable acids or bases, and the prodrugs thereof.

4. A compound of the general formula (I)
A-B-D-E-G-K-L (I),
in which
A stands for H or H—(R^{A1})i^{A}
in which
R^{A1} denotes 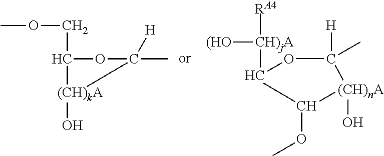
in which
R^{A4} denotes H, or COOH,
ᵢA is 1 to 6,
ⱼA is 0 or 1,
ₖA is 2 or 3,
ₙA is 1 or 2,
the groups R^{A1} being the same or different when ᵢA is greater than 1;
B denotes 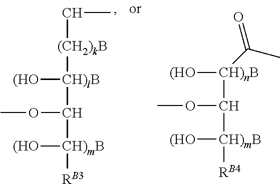
A-B stands for 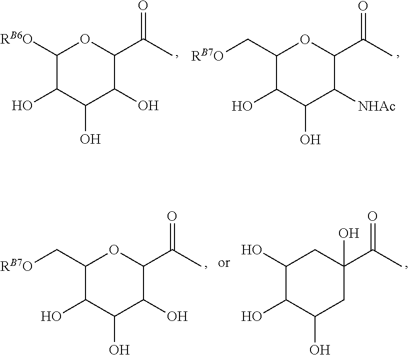
in which
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3,
ₙB is 1, 2, or 3,
R^{B6} denotes C_{IA} alkyl, phenyl, or benzyl, and
R^{B7} denotes H, C₁₋₄ alkyl, phenyl, or benzyl,
D stands for 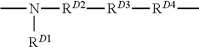
in which
R^{D1} denotes H or C₁₋₄ alkyl,
R^{D2} denotes a bond or C₁₋₄ alkyl,
R^{D3} denotes 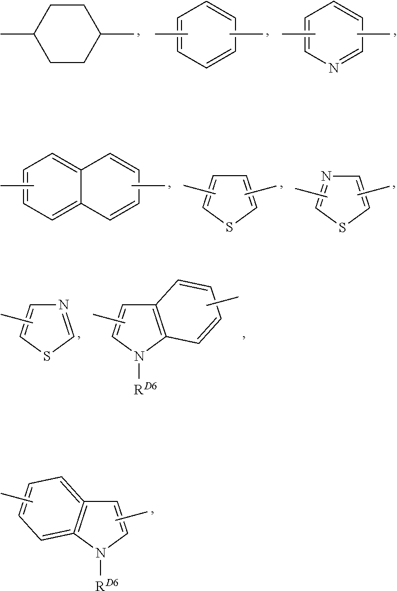
in which
R^{D4} denotes a bond, C₁₋₄ alkyl, CO, SO₂, or —CH₂—CO, and
R^{D6} denotes H or CH₃,
E stands for 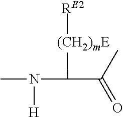
in which
ₘE is 0 or 1,
R^{E2} denotes H, C₁₋₅ alkyl, or C₃₋₈ cycloalkyl, which groups may carry up to three identical or different substituents selected from the group consisting of C₁₋₄ alkyl, OH, O—CH₃, F, and Cl;
G stands for 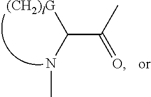
where ₗG is 2, 3, or 4 and one of the CH₂ groups in the ring is replaceable by O, S, NH, or N(C₁₋₃ alkyl), 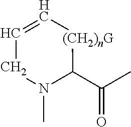
in which
ₙG is 0 or 1;
K stands for
NH—CH₂-Q^{K}
in which
Q^{K} denotes 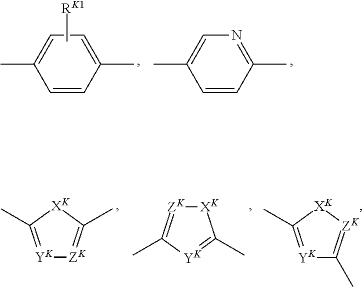
in which
R^{K1} denotes H, CH₃, OH, O—CH₃, F, or Cl,
X^{K} denotes O, S, NH, N—CH₃,
Y^{K} denotes ═CH—, 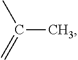
or ═N—,
Z^{K} denotes ═CH—, 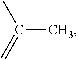
or ═N—,
L stands for 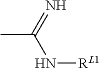
in which
R^{L1} denotes H, OH, or CO₂—(C₁₋₆ alkyl),
and the tautomers thereof, stereoisomers thereof, salts thereof with pharmacologically acceptable acids or bases, and the prodrugs thereof.

5. A compound of the general formula (I)
A-B-D-E-G-K-L (I),
in which
A stands for H or H—(R^{A1})i^{A}
in which
R^{A1} denotes 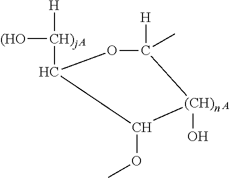
in which
ᵢA is 1 to 6,
ⱼA is 0 or 1,
ₙA is 1 or 2,
the groups R^{A1} being the same or different when ᵢA is greater than 1;
B denotes 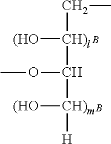
in which
ₗB is 1, 2, or 3,
ₘB is 1 or 2,
D stands for a bond,
E stands for 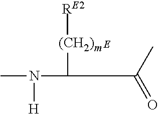
in which
ₘE is 0 or 1,
R^{E2} denotes H, C₁₋₆ alkyl, C₃₋₈ cycloalkyl, phenyl, diphenylmethyl, or dicyclohexylmethyl,
the building block E preferably exhibiting D configuration,
G stands for 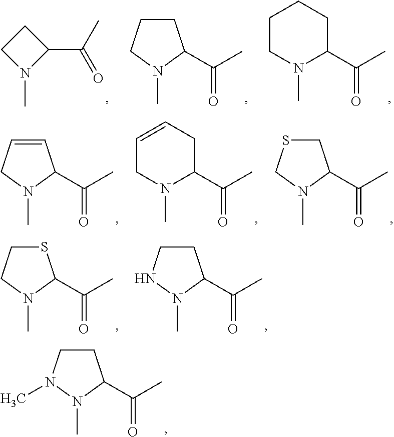
building block G preferably exhibiting L configuration,
K stands for
NH—CH₂-Q^{K}
in which
Q^{K} denotes 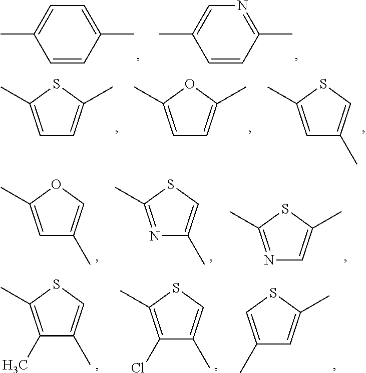
L stands for 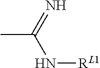
in which
R^{L1} denotes H, OH, or CO₂—(C₁₋₆ alkyl),
and the tautomers thereof, stereoisomers thereof, salts thereof with pharmacologically acceptable acids or bases, and the prodrugs thereof.

6. A compound of the general formula (I)
A-B-D-E-G-K-L (I),
in which
A stands for H or H-(R^{A1})i^{A}
in which
R^{A1} denotes 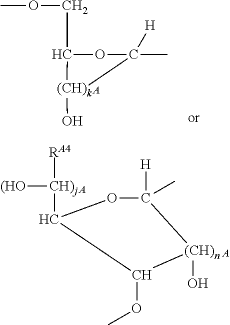
in which
R^{A4} denotes H, or COOH,
ᵢA is 1 to 6,
ⱼA is 0 or 1,
ₖA is 2 or 3,
ₙA is 1 or 2,
the groups R^{A1} being the same or different when ᵢA is greater than 1;
B denotes 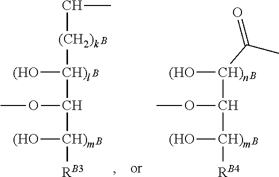
A-B stands for 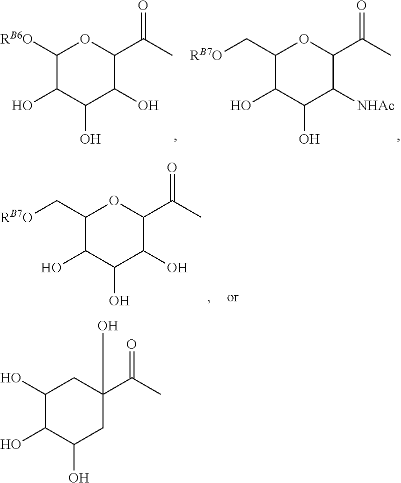
in which
R^{B3} denotes H, CH₃, or COOH,
R^{B4} denotes H, CH₃, COOH, or CHO, in which latter case intramolecular acetal formation may take place,
ₖB is 0 or 1,
ₗB is 1, 2, or 3,
ₘB is 0, 1, 2, or 3,
ₙB is 1, 2, or 3,
R^{B6} denotes C₁₋₄ alkyl, phenyl, or benzyl, and
R^{B7} denotes H, C₁₋₄ alkyl, phenyl, or benzyl,
D stands for 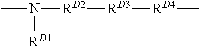
in which
R^{D1} denotes H,
R^{D2} denotes a bond or C₁₋₄ alkyl,
R^{D3} denotes 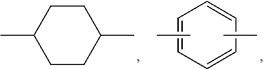
R^{D4} denotes a bond, C₁₋₄ alkyl, CO, SO₂, or —CH₂—CO, and
E stands for 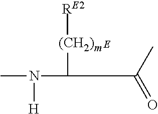
in which
ₘE is 0 or 1,
R^{E2} denotes H, C₁₋₆ alkyl, or C₃₋₈ cycloalkyl, which groups may carry up to three identical or different substituents selected from the group consisting of F and Cl;
G stands for 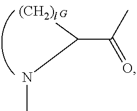
where ₗG is 2 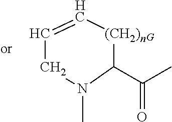
in which
ₙG is 0,
K stands for
NH—CH₂-Q^{K}
in which
Q^{K} denotes 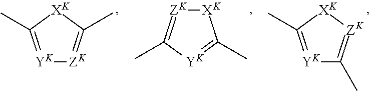
in which
X^{K} denotes S,
Y^{K} denotes ═CH—, or ═N—,
Z^{K} denotes ═CH—, or ═N—,
L stands for 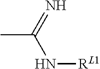
in which
R^{L1} denotes H, or OH,
and the tautomers thereof, stereoisomers thereof, salts thereof with pharmacologically acceptable acids or bases, and the prodrugs thereof.

7. A medicinal drug comprising at least one compound of claim 1.

8. A method of using one or more compounds of claim 1 for the preparation of medical drugs for the treatment or prophylaxis of diseases which can be alleviated by inhibition of one or more serine proteases.

9. A method as defined in claim 8, wherein the serine protease for a compound is thrombin.

10. A method as defined in claim 8, wherein the serine protease for a compound is C1s or C1r.
